(19) United States
(12) Patent Application Publication
(10) Pub. No.: US 2023/0173059 A1
(43) Pub. Date: Jun. 8, 2023

(54) TOLL-LIKE RECEPTOR (TLR) AGONIST NANOPARTICLES AND USES THEREOF

(71) Applicant: The Board of Trustees of the Leland Stanford Junior University, Stanford, CA (US)

(72) Inventors: Eric A. Appel, Stanford, CA (US); Emily C. Gale, Stanford, CA (US); Anton A. A. Smith, Stanford, CA (US); Gillie A. Roth, Stanford, CA (US); Vittoria C.T.M. Picece, Stanford, CA (US)

(73) Assignee: The Board of Trustees of the Leland Stanford Junior University, Stanford, CA (US)

(21) Appl. No.: 17/925,443

(22) PCT Filed: May 14, 2021
(86) PCT No.: PCT/US2021/032575

§ 371 Date: Nov. 15, 2022

Related U.S. Application Data
(60) Provisional application No. 63/025,845, filed on May 15, 2020.

(51) Int. Cl. A61K 39/215; A61K 47/69; A61K 39/395; A61P 31/14

(52) U.S. Cl. CPC A61K 39/215; A61K 47/6937; A61K 39/3955; A61P 31/14; B82Y 5/00

(57) ABSTRACT

The present disclosure provides nanoparticles comprising a polymer and a plurality of TLR agonist moieties conjugated to the polymer and present on the surface of the nanoparticles. Methods of producing the nanoparticles, hydrogels comprising the nanoparticles, and vaccines comprising the nanoparticles and/or hydrogels are also provided. Methods for inducing an antigen-specific humoral immune response or enhancing cancer immunotherapy in a subject are also provided.

### CROSS-REFERENCE

This application claims the benefit of U.S. Provisional Application No. 63/025,845, filed on May 15, 2020, and which is incorporated in its entirety herein by reference.

### BACKGROUND

Clinical limitations of TLR agonist therapies are due to severe systemic toxicity and a narrow therapeutic window. Immunostimulatory molecules, like the TLR7/8 agonist Resiquimod (R848), have shown incredible promise as adjuvants in both prophylactic vaccines and cancer immunotherapies. Unfortunately, R848 has been limited clinically due to extreme systemic toxicity that results from the lack of pharmacokinetic control of the small hydrophobic drug. As such, there is a need for a delivery platform to overcome these limitations. The present invention satisfies this need and provides related advantages as well.

### BRIEF SUMMARY

In some aspects, the present disclosure provides a nanoparticle comprising a polymer and a plurality of TLR agonist moieties conjugated to the polymer, wherein the plurality of TLR agonist moieties is present on the surface of the nanoparticle.

In some embodiments, the plurality of TLR agonist moieties is a plurality of TLR7/8 agonist moieties. In some embodiments, the plurality of TLR agonist moieties comprises a 1H-imidazo[4,5-c]quinolone core structure. The plurality of TLR agonist moieties may comprise resiquimod (R848), imiquimod, gardiquimod, or mixtures thereof. In some embodiments, the plurality of TLR7/8 agonist moieties comprises a plurality of N-(4-((4-amino-2-(ethoxymethyl)-1H-imidazo[4,5-c]quinolin-1-yl)methyl)benzyl)-3-(prop-2-yn-1-yloxy)propanamide moieties.

In some embodiments, the plurality of TLR agonist moieties is a plurality of TLR9 agonist moieties. The plurality of TLR9 agonist moieties may comprise a plurality of cytidine-phosphate-guanosine (CpG) moieties.

In some embodiments, the polymer comprises poly(ethylene glycol)-b-poly(lactic acid) (PEG-PLA). In some embodiments, the nanoparticle is up to about 200 nm in diameter. In some instances, the nanoparticle is about 50 nm in diameter. In other instances, the nanoparticle is about 30 nm in diameter. In some embodiments, the nanoparticle further comprises a plurality of mannose moieties conjugated to the polymer. In certain instances, the polymer is conjugated to the plurality of TLR agonist moieties via a 1,2,3-triazole linkage.

In other aspects, the present disclosure provides a method of producing a nanoparticle described herein, the method comprising:
(a) conjugating the polymer with the plurality of TLR agonist moieties to form a conjugated polymer;
(b) mixing an amount of the conjugated polymer and an amount of unconjugated polymer at a ratio to achieve a target density of the plurality of TLR agonist moieties on the surface of the nanoparticle; and
(c) precipitating the nanoparticle from the mixture.

In some embodiments, the polymer comprises an azide terminal group and the plurality of TLR agonist moieties comprises an alkyne derivative thereof.

In further aspects, the present disclosure provides a hydrogel comprising a nanoparticle described herein. In some embodiments, the hydrogel comprises optionally hydrophobically-modified hydroxypropyl methylcellulose (HPMC).

In related aspects, the present disclosure provides a vaccine comprising a nanoparticle and/or hydrogel as described herein. In some embodiments, the vaccine comprises one or more subunit antigens. In some embodiments, the antigen comprises a viral antigen, a bacterial antigen, a fungal antigen, or a protozoan antigen. In certain instances, the viral antigen is an antigen from a virus such as, e.g., a coronavirus (e.g., SARS-CoV or SARS-CoV-2), an influenza virus, a human immunodeficiency virus (HIV), a human papillomavirus (HPV), a porcine circovirus (PCV), etc. In certain instances, the protozoan antigen is from a parasite that causes a disease such as malaria. In particular embodiments, the viral antigen comprises a SARS-CoV-2 subunit antigen.

Methods for inducing an antigen-specific humoral immune response in a subject comprising administering a vaccine described herein and methods for enhancing cancer immunotherapy in a subject comprising administering a nanoparticle or hydrogel described herein are also provided. In embodiments related to enhancing cancer immunotherapy, the nanoparticle or the hydrogel (i.e., a nanoparticle-loaded hydrogel) can be co-administered with an immune checkpoint inhibitor, an immunomodulatory molecule, or a combination thereof. In certain instances, the immune checkpoint inhibitor is a checkpoint antibody (e.g., PD1, PDL1, CTLA4, OX40, etc.). In certain instances, the immune checkpoint inhibitor is an antibody that prevents interactions of CTLA4/(CD80/CD86) and PD1/PD-L1. In certain instances, the immunomodulatory molecule is a cytokine (e.g., IL2, IL 12, IL13, IL 15, etc.) or a chemokine.

### BRIEF DESCRIPTION OF THE DRAWINGS

FIGS. 1A-B: Schematic representation of TLR7/8A NP delivery or soluble TLR7/8a delivery in a subcutaneous murine tumor model. FIG. 1A: Peritumoral, subcutaneous TLR7/8a NP delivery results in lower levels of circulating cytokines (brown circles), greater draining to lymph nodes, and likely potent activation of TLR7/8 receptors in APCs (purple cells) like DCs and macrophages. The altered cytokine profile and enhanced drainage of TLR7/8a to lymph nodes significantly slows tumor growth, extends survival, and causes less systemic toxicity. FIG. 1B: Peritumoral, subcutaneous soluble TLR7/8a delivery results in increased levels of many circulating cytokines, non-specific diffusion throughout the body, and likely less APC activation in lymph nodes. This delivery route leads to greater systemic toxicity and weaker anti-tumor effects.

FIGS. 2A-2B: PEG-PLA NP as a tunable platform. FIG. 2A: Schematic representation showing N₃-PEG-PLA block copolymers can be modified alkyne-azide click chemistry with either mannose or an R848 analog, TLR7/8a. FIG. 2B: Schematic demonstrating that mixing PEG-PLA with different termini at various ratios allows for simple manufacturing of nanoparticles with control of surface presentation of conjugated moieties.

FIGS. 3A-3E: Evaluation of high valency TLR7/8a NP. FIGS. 3A-3B: Activity graphs across a range of TLR7/8a concentrations (0.08-10 μg/mL) delivered on NPs at different densities, on NPs with or without mannose, or in the soluble form. Absorbance at 655 nm is a reporter output for TLR activation in the RAW-Blue murine macrophage reporter cell line (Invivogen) used in 2A-2B. FIG. 3C: GraphPad Prism software was used to determine EC₅₀ values (using a log(agonist) vs. response) and maximum absorbance values for each activation curve. FIG. 3D: ELISA analysis of IFNa in serum 3-18 hours after intraperitoneal administration of NP or soluble TLR7/8a treatments (n=3). Data depict mean±SD; values were analyzed by ordinary one-way ANOVA with multiple comparisons to the control group. *p<0.05, **p<0.01. FIG. 3E: Area under the curve (AUC) of IFNa in serum from 3-18 hours (n=3). Data depict mean±SD; values were analyzed by ordinary one-way ANOVA with multiple comparisons to the control group. *p<0.05, **p<0.01.

FIGS. 4A-4H: Treatment in a murine colon adenocarcinoma model (MC38). FIG. 4A: Timeline for murine colon adenocarcinoma (MC38) inoculation and treatment. Mice were inoculated with half a million MC38 cells on SC on the right flank. Measurement and treatments began 8 days later when tumors were palpable. Treatments were given 4 times over 8 days. Mice were euthanized when tumors reached 150 mm². FIGS. 4B-4E: Tumor growth curves over time for individual mice that received PBS injections (FIG. 4B), IP aPD-L1 treatment (FIG. 4C), IP aPD-L1 and SC soluble TLR7/8a treatment (FIG. 4D), IP aPD-L1 and SC NP TLR7/8a treatment (FIG. 4E) (n=8). FIG. 4F: Average tumor growth for each treatment group for the first 10 days following the start of treatment (n=8). Data depict mean±SEM: values were analyzed by ordinary one-way ANOVA at day 10 with multiple comparisons to the control group. *p<0.05, **p<0.01. FIG. 4G: Survival curves showing percent survival over the duration of the study of all treatment groups. FIG. 4H: Mean survival for each treatment group. Survival means are shown as inverse link transformed least squares mean±SE. Tukey Kramer post-hoc tests were used to correct for multiple comparisons.

FIGS. 5A-5F: Mice toxicity assessment of TLR7/8a treatment. FIG. 5A: Timeline for murine colon adenocarcinoma (MC38) inoculation and serum collection. Blood for Luminex analysis was collected 2 hours after the first treatment. FIG. 5B: Change in mouse body mass over the course of treatment (n=8). Young, healthy mice should gain about 5% of their body mouse each week (see, Body Weight Information for C57BL/6J. https://www.jax.org/jax-mice-and-services/strain-data-sheet-pages/body-weight-chart-000664). Data depict mean±SEM; values were analyzed by ordinary one-way ANOVA at day 7 with multiple comparisons to the control group. FIG. 5C: Heatmap depiction of mean MFI cytokine levels in mouse serum as determined by Luminex (n=3). P-values are presented in Table 4 of Example 1. FIG. 5D: Levels of select cytokines that promote growth, activation, and differentiation (n=3). FIG. 5E: Levels of select proinflammatory cytokines in serum (n=3). FIG. 5F: Levels of select chemokines in serum (n=3). Data in FIGS. 5D-5F depict mean±SD; values were analyzed by t test. *p<0.5. **p<0.01, ***p<0.001.

FIGS. 6A-6B: Synthesis of conjugates. FIG. 6A: Synthetic scheme for IV and Mannose-PEG-PLA conjugate (C). ¹H NMR data is shown in the bottom panel for IV (D₂O) and C (d₆-DMSO). FIG. 6B: Synthetic scheme for III and TLR 7/8a-PEG-PLA conjugate (D). ¹H NMR data is shown in the bottom panel for III (CDCl₃) and D (CDCl₃).

FIGS. 7A-7B: Conjugate characterization. FIG. 7A: TLR7/8a conjugation confirmed by SEC. UV-SEC traces of N₃-PEG-PLA and TLR7/8-PEG-PLA was compared with UV absorbance normalized to the peaks RI intensities. FIG. 7B: TLR7/8-PEG-PLA shows strong UV absorption at 280 nm, with no absorption from N₃-PEG-PLA.

FIGS. 8A-8C: A schematic representation of the PNP hydrogel and proposed in vivo response to prolonged hydrogel-based vaccine delivery. FIG. 8A: Vaccine-loaded PNP hydrogels are formed when dodecyl-modified hydroxypropylmethylcellulose (HPMC-C₁₂) is combined with poly(ethylene glycol)-b-poly(lactic acid) (PEG-PLA) nanoparticles and vaccine cargo, including ovalbumin (OVA) and Poly(I:C). Multivalent and dynamic non-covalent interactions between the polymer and nanoparticles constitute physical cross-links within the hydrogel structure. FIG. 8B: After subcutaneous (SC) injection of the hydrogel vaccine, local and migratory immune cell such as neutrophils and APCs infiltrate the gel, become activated, and then (i) activated APCs may migrate to the draining lymph nodes. The gel provides (ii) sustained release of the vaccine cargo to the draining lymph nodes, prolonging the germinal center response. FIG. 8C: The extended antigen availability in the germinal centers leads to increased (i) somatic hypermutation (SHM) and (ii) affinity selection, ultimately promoting higher affinity antibodies and a strong humoral immune response.

FIGS. 9A-9K Material characterization and dynamics of entrapped molecular cargo. FIG. 9A: Frequency-dependent (σ=1.8 Pa, 25° C.) oscillatory shear rheology FIG. 9B: steady shear rheology of two PNP hydrogel formulations designated “1:5” and “2:10”. FIG. 9C: Yield stress values from stress ramp measurements (n=3). FIG. 9D: Step-shear measurements of 1:5 and 2:10 gels over two cycles with alternating high shear (100 s⁻¹) and low shear (0.05 s⁻¹) rates. FIG. 9E: Images of 2:10 gel injection through a 21-gauge needle showing (i) before injection, (ii) during injection, (iii-iv) and after injection. FIG. 9F: FRAP experiment showing photobleaching of a select area at 0 sec, and the fluorescence recovering as fluorescent molecules diffuse back into the select area. FIG. 9G: Ratio of the diffusivity of OVA to the diffusivity of Poly(I:C) calculated from R_{H} values for PBS or using FRAP for the 1:5 and 2:10 gels. Values closer to one indicate more similar diffusivities of the OVA and Poly(I:C) (n=3). FIG. 9H: Ratio of the diffusivity of the cargo (OVA or Poly(I:C)) to the self-diffusivity of the hydrogel network. Values closer to one indicate that cargo diffusivity is limited by self-diffusion of the hydrogel (n=3). FIG. 9I: Representative schematic of (i) the 1:5 gel with OVA moving quickly and Poly(I:C) and the hydrogel matrix diffusing slower, and (ii) the 2:10 gel with the OVA, Poly(I:C), and hydrogel matrix all diffusing slowly. FIG. 9J: Gel mass over time following SC implantation measured from in vivo explants (n=5). FIG. 9K Fluorescence of Alexa-647-OVA retained in explanted gels fit with an exponential decay to calculate tin (n=5). All error bars are mean±s.d., P value determined by two-tailed t-test.

FIGS. 10A-10G: Antibody concentration and affinity following immunization. FIG. 10A: Timeline of the experimental setup shows SC injection of a model vaccine containing OVA and Poly(I:C) in a gel or bolus formulation at day 0, antibody analysis over time following a single administration, boost with a bolus vaccine formulation at day 40 or 90, and analysis of the immune response 15 days after the boost. FIG. 10B: Serum anti-OVA IgG1 concentrations from day 0 to day 90 after a single injection of vaccines (n=5 to 19; 1 to 4 independent experiments; mean±s.e.m.). ***p<0.001 and ****p<0.0001 compared to bolus, #p<0.05, # #p<0.005 compared to 1:5, determined by mixed-effects analysis with Tukey's post hoc test. FIGS. 10C-10D: Serum anti-OVA IgG1 concentrations 15 days after bolus boost on day (FIG. 10C) 40 or (FIG. 10D) 90 for animals receiving either bolus, 1:5 gel, and 2:10 gel vaccines (n=4 to 5; mean±s.d.). Reported P values determined by one-way ANOVA with Tukey's post hoc test. FIG. 10E Model comparing competitive binding data with K_{D} ranging from 1 to 104 nM. FIG. 10F: Representative competitive binding curves for bolus, 1:5 gel, and 2:10 gel vaccine groups after the day 90 boost compared to a mAb reference competing with the same mAb. FIG. 10G: Calculated K_{D} values from fitted binding curves for 2:10 gel and bolus vaccine groups (n=4; mean±s.d.). P values determined by one-way ANOVA with Tukey's post hoc test.

FIGS. 11A-11J: Characterization of the local inflammatory niche. FIG. 11A: Schematic of the inflammatory niche within the gel depot and an experimental flow chart. FIG. 11B: Picture of surgical removal of 2:10 gel after 7 days in vivo. FIG. 11C: Total cells in 2:10 gel with or without vaccine (OVA+Poly(I:C)) were quantified using flow cytometry. FIGS. 11D-11G: Total count of neutrophils (FIG. 11D), monocytes (FIG. 11E), macrophages (FIG. 11F), and DCs (FIG. 11G) found in the empty and vaccine-loaded 2:10 gels. FIG. 11H: The frequency of cDC1 (XCR1^{hi}CD11b^{lo}) and cDC2 (XCR1^{lo}CD11b^{hi}) of the total dendritic cells (DCs) in the vaccine-loaded 2:10 gel. FIG. 11I: Histogram of the Alexa-647 OVA signal in cDC2s from individual mice with and without the vaccine. FIG. 11J: The frequency of neutrophils, monocytes, macrophages. DCs, other myeloid cells, and non-myeloid cells within the CD45⁺ cell populations found in the empty and vaccine-loaded 2:10 gels. For FIGS. 11C-11J, n=3 mice. All error bars are mean±s.d., P values determined by two-tailed t-test.

FIGS. 12A-12H: Germinal center response to single vaccine administration. FIG. 12A: Immunohistochemistry (IHC) of explanted inguinal lymph node 15 days after OVA+Poly(I:C) vaccine administration in 2:10 and bolus groups to visualize germinal centers (red) and naïve B cells (green). FIG. 12B-12C: The frequency of GCBCs within total B cells at day 15 (FIG. 12B) and day 30 (FIG. 12C) after prime. FIG. 12D-12E: frequency of IgG1+GCBCs within total GCBCs at day 15 (FIG. 12D) and day 30 (FIG. 12E) after prime in the inguinal lymph nodes were measured by flow cytometry (n=5 to 10). FIG. 12F: Schematic of the GC response. FIG. 12G: The percent of Tfh cells out of the CD4+ cell population. FIG. 12H: the ratio of LZ to DZ GCBCs in the inguinal lymph nodes at day 15 after vaccination (n=5 to 10). For FIGS. 12B, 12D, and 12H data come from 2 independent experiments, all other graphs represent 1 independent experiment. All error bars are mean±s.d., P values determined by one-way ANOVA with Tukey's post hoc test.

FIGS. 13A-13J: Influenza vaccine formulation and antibody response. FIG. 13A: TLR 7/8 nanoparticles (TLR 7/8 NP) are synthesized from poly(ethylene glycol)-b-poly(lactic acid) conjugated to a TLR 7/8 agonist (purple) and then formulated with HPMC-C₁₂ and hemagglutinin (HA) to create an influenza vaccine (2:10 with TLR 7/8 NP). FIG. 13B: Frequency-dependent (σ=1.8 Pa, 25° C.) oscillatory shear rheology. FIG. 13C: steady shear rheology of the 2:10 gel with TLR 7/8 NP. FIG. 13D: Serum anti-HA IgG titers from day 7 or 14 to day 140 after single injection of HA delivered in the 2:10 gel with TLR 7/8 NP, an MF59 bolus, or Alum bolus. P values for 2:10 with TLR 7/8 NP compared to Alum (top, gray) or MF59 (bottom, green) are shown (n=4 to 5). FIG. 13E: Area under the curve (AUC) of anti-HA IgG titers (n=4 to 5). Serum anti-HA IgG1 (FIG. 13F), IgG2b (FIG. 13G), and IgG2c (FIG. 13H) titers from day 56 after single injection of HA delivered in the 2:10 gel with TLR 7/8 NP, an MF59 bolus, or Alum bolus. FIG. 13I: Anti-HA titers for A/California/07/2009(H1N1). FIG. 13J: A/Michigan/45/2015 (H1N1) for serum from day 56 after single injection of A/Brisbane/59/2007 (H1N1) HA delivered in the 2:10 gel with TLR 7/8 NP or as a bolus with MF59. All error bars are mean±s.d., P values determined by two-way ANOVA with Tukey's post hoc test (FIG. 13D), one-way ANOVA with Tukey's post hoc test (FIG. 13E-13H), or a two-tailed t-test (FIG. 13I and FIG. 13J).

FIGS. 14A-14B: Batch to batch consistency of rheological characterization. FIG. 14A: Replicates of frequency-dependent (σ=1.8 Pa, 25° C.) oscillatory shear rheology. FIG. 14B: steady shear rheology of 2:10 gels (n=3) demonstrating batch to batch consistency.

FIG. 15: Raw data for yield stress determination. Representative stress ramp rheological experiments for 1:5 and 2:10 gels with the peak viscosity indicated by a red line showing how the yield stress was measured.

FIGS. 16A-16B: A representative fluorescence recovery after photobleaching (FRAP) experiment. FRAP was used to characterize the mobility of the cargo and polymer through the hydrogel systems. FIG. 16A: Several frames using a low light level are acquired to determine the initial fluorescence, and then a high intensity of light is applied for a short time inside a region of interest to bleach the fluorescence in the sample. Finally, the recovery of fluorescence is monitored to measure how fast the molecule of interest redistributes. FIG. 16B: The figure shows raw data probing the self-diffusion of HPMC within the weak hydrogels.

FIGS. 17A-17C: Cargo diffusivity schematics and values. FIG. 17A: Representations of the diffusivity of OVA (43 kDa) and Poly(I:C) (>1 MDa) in (i) PBS, (ii) a covalently crosslinked PEG hydrogel, (iii) 1:5 PNP gel, and (iv) 2:10 PNP gel. FIG. 17B: The ratio of the diffusivity of OVA to the diffusivity of Poly(I:C), where values closer to one indicate more similar diffusivities. FIG. 17C: absolute OVA diffusivities in each matrix. PBS diffusivities were calculated from R_{H} values using the Stokes-Einstein equation, and PEG diffusivities were calculated with R_{H} values using a multiscale diffusion model (see, Axpe, E. el al. A Multiscale Model for Solute Diffusion in Hydrogels. Macromolecules 52, 6889-6897, (2019)), while the 1:5 and 2:10 gel diffusivities were determined using FRAP experiments described in FIG. 9F.

FIGS. 18A-18B: Antibody concentrations after prime and boost for various vaccine formulations. FIG. 18A: Serum anti-OVA IgG1 concentrations from day 0 to day 42 or 45 after prime of vaccines (n=5 to 19) delivered in different formulations (Error bars, mean±s.e.m.): (i) OVA and Poly(I:C) in a 2:10 gel, (ii) OVA and Poly(I:C) with PEGPLA NPs, (iii) OVA alone in a 2:10 hydrogel, (iv) OVA and Poly(I:C) in separate 2:10 gels administered on contralateral flanks, (v) OVA alone in PBS, and (vi) OVA and Poly(I:C) in PBS. FIG. 18B: Serum anti-OVA IgG1 concentrations 15 days after a day 45 boost (Error bars, mean±s.d.). All groups were boosted with a bolus vaccine with OVA and Poly(I:C) to assess their responsiveness on the same timeframe. These results show that co-delivery of OVA with the Poly(I:C) adjuvant significantly increases the humoral immune response. Statistical analysis, *p<0.05, **p<0.01 with one-way ANOVA.

FIGS. 19A-19F: Characterization of IgG subclasses in OVA+Poly(I:C) vaccine post-boost. Serum anti-OVA IgG1 (FIG. 19A), IgG2b (FIG. 19B), and IgG2c (FIG. 19C) concentrations for the bolus, 1:5 gel, and 2:10 gel vaccine groups 15 days after a day 90 bolus boost. Data reorganized for the bolus (FIG. 19D), 1:5 gel (FIG. 19E), and 2:10 gel (FIG. 19E) to show additional statistical comparisons. P values determined by one-way ANOVA with Tukey's test.

FIG. 20: Anti-PEG antibody response after OVA+Poly(I:C) vaccine after boost. Anti-PEG IgG in serum 15 days after bolus boost on day 90 for bolus, 1:5 gel, and 2:10 gel primes along with naïve serum for comparison. All samples were either below or near the detection limit of the assay and no significant differences were found between treatments and naïve serum. P values determined by one-way ANOVA with Tukey's test.

FIG. 21: Long-term biocompatibility in subcutaneous space. Images of the subcutaneous space 8 weeks after OVA+Poly(I:C) vaccine administration for the bolus, 1:5 gel, and 2:10 gel treatment groups. Images indicate no noticeable vascularization or fibrotic response differences. The hydrogel materials were completely degraded by this time point.

FIGS. 22A-22C: Competitive binding assay with serum 15 days after a day 90 boost. Individual binding curves for all bolus (FIG. 22A), 1:5 (FIG. 22B), and 2:10 (FIG. 22C) samples with the competitive binding fits used to calculate the K_{D}.

FIGS. 23A-23B: Surface plasmon resonance (SPR) affinity analysis of anti-OVA serum antibodies post-boost. FIG. 23A: OVA dissociation from serum antibodies measured by SPR after a day (i) 40 or (ii) 90 boost (n=4). FIG. 23B: K_{eq} values for high-affinity antibodies from SPR for a day (i) 40 and (ii) 90 boost using the lowest k_{d} from a 3-decay fit of the dissociation and the measured kₐ (n=4 to 5). Severe limitations arise in measuring dissociation times with this instrument, which can only collect dissociation data for roughly 30 min (corresponding to k_{d} values of ˜10⁻⁴ s⁻¹, and thus K_{eq} values of ˜10⁸ M⁻¹) before baseline drift introduces significant error into the measurement. These data, therefore, serve primarily to verify the trends observed with the more accurate competitive binding assay reported in FIGS. 10E-10G. P values determined by one-way ANOVA and Tukey's post hoc test.

FIG. 24: Representative gating strategy for gel infiltration analysis. Neutrophils were defined as CD45+CD19− CD3− Ly6G+. Dendritic cells were defined as CD45+CD19− CD3− Ly6G− MHCII+CD11C+ with cDC1s as XCR1^{hi} CD11b^{lo} and cDC2s as XCR1^{lo} CD11b^{hi}. Monocytes were defined as CD45+CD19− CD3− Ly6G− CD11b+ Ly6C+. Macrophages were defined as CD45+CD19− CD3− Ly6G− CD11b+ Ly6C− CD64+.

FIG. 25: Representative gating strategy for GC analysis. GCBCs were defined as CD19+CD95+GL7+.

FIGS. 26A-26B: Synthesis of TLR7/8 agonist PEG-PLA conjugate. FIG. 26A: NHS coupling of TLR7/8 agonist to alkyne (I), and the coupling to azide-terminated PEG-PLA (II) to make PEG-PLA with the TLR7/8 agonist presenting on the PEG terminus of the block copolymer (III). FIG. 26B: ¹H-NMR spectrum of TLR7/8 agonist alkyne (I) stacked with TLR7/8 agonist PEG-PLA conjugate (III). Broadening of peaks corresponds to the TLR7/8 ligand and the emergence of the triazole proton, a, along with disappearance of terminal alkyne proton, 15, confirms conjugation and the formation of III.

FIG. 27: Anti-HA antibody response for various formulations. Serum anti-HA IgG titers from day 7 or 14, to day 56 after single injection of HA delivered in various formulations: (i) alongside TLR 7/8 NPs as a bolus, (ii) alongside soluble TLR 7/8 (R848) as a bolus. (iii) alone (i.e., without adjuvant) as a bolus, (iv) loaded into the 2:10 gel with a soluble TLR7/8 agonist (R848), or (v) loaded into the 2:10 gel with TLR7/8 NPs.

FIGS. 28A-28B: Differences between HA in influenza strains of interest. FIG. 28A: Phylogenetic tree of the influenza strains used in these studies based on HA DNA sequence where the branch lengths represent modifications per site. Tree was created using the ‘Generate Phylogenetic Tree’ tool on the NIAID Influenza Research Database (IRD) [Zhang Y, et al. (2017)] through the web site at http://www.fludb.org. FIG. 28B: Comparison of A/Brisbane/59/2007(H1N1) hemagglutinin DNA sequence or amino acid (AA) sequence with the sequences of other strains of interest; A/Michigan/45/2015 (Mich15). A/California/07/2009 (Cal09), and A/Puerto Rico/8-WG/1934 (PR8). Values were found using BLAST (https://blast.nchi.nlm.nih.gov/).

FIGS. 29A-29C: Characterizing protective antibodies with a passive transfer influenza challenge model. FIG. 29A: Serum collected from 5 mice per group at day 56 after vaccine prime was pooled together and antibodies were isolated. The antibodies were incubated with PR8 influenza virus for 30 minutes at 37° C. before nasal inoculation into mice. Mouse weight was monitored daily until weight loss exceeded 25% of original weight. FIG. 29B: Normalized mass curves for animals receiving antibodies from each vaccine treatment group: (i) naïve serum. (ii) TLR 7/8 NP-based 2:10 hydrogels, (iii) MF59, and (iv) Alum. Each line represents an individual animal and data was collected until mice were sacrificed or they returned to original weight. FIG. 29C: Survival of mice to 75% of original weight shows that both the hydrogel vaccine and MF59 vaccine lead to significantly increased survival compared to the naïve serum treatment, indicating that the serum from the vaccinated mice contained protective antibodies. P values were determined by Log-rank (Mantel-Cox) test.

FIGS. 30A-30B: Schematic representation of a subcutaneous vaccine injection in mice and model for in vivo release. (FIG. 30A) Delivery of CpG adjuvant can be achieved in different ways: as a free species, tethered to PEG-PLA NPs or tethered to NPs and encapsulated in polymer-nanoparticle (PNP) hydrogels. PNP hydrogels are loaded with vaccine cargo, including antigen and adjuvant (CpG-NPs), and allow for sustained vaccine exposure. After subcutaneous injection of the hydrogel vaccine, vaccine components can be transported to the lymph nodes (LNs) either by drainage through antigen presenting cells (APCs) that have previously infiltrated the gel, or by LN drainage of the single vaccine components themselves. (FIG. 303) CpG-NPs of different valencies, from 10 to 50% are synthesized; an increase in NPs valency corresponds to an increase in CpG density on the NPs surface.

FIGS. 31A-31G: Synthetic procedure and characterization of TLR9 functional NPs. (FIG. 31A) Synthetic scheme for the fabrication of CpG functionalized NPs. (I) ROP for the synthesis of PEG-b-PLA and N₃-PEG-b-PLA, PEG-b-PLA with an azide as the end group (red half circle) on the hydrophilic PEG terminus. (II) Formation of azide-functionalized NPs (N₃-NPs) via nanoprecipitation of polymer solutions in water. (III) NHS coupling of cyclooctyne functionality (DBCO-PEG4-NHS ester) to NH₂-CpG yields DBCO-CpG (blue half circle). (IV) Copper-free click reaction between N₃-NPs and DBCO-CpG yields CpG-functionalized (purple circle) NPs. (FIG. 31B) Investigation of influence of DBCO-CpG molar excess on the click reaction conversion. Three equivalents of DBCO-CpG result in reaction conversions higher than 90%. (FIG. 31C) Normalized UV absorbance of 10%, 20%, 30% and 50% Class C CpG NPs (CpG-C NPs). The increase in UV absorbance is a result of higher CpG valency. (FIG. 31D) GPC traces of 30% CpG-C NPs before (3 molar excess of DBCO-CpG) and after purification through a SEC column. Disappearance of the DBCO-CpG peak at 23 min confirms complete removal of the unconjugated free CpG specie. (FIG. 31E) Surface zeta potential measurements of bare PEG-b-PLA NPs and different CpG-C NPs valencies in PBS. Decreasing NPs surface potential with increasing CpG valency confirms larger CpG conjugation with increased valency (n=3). (FIG. 31F) Characteristic dynamic light scattering (DLS) curves for N₃-NPs (grey), 30% CpG-B NPs (orange) and 30% CpG-C NPs (dark blue) in PBS (n=3-9). (FIG. 31G) Hydrodynamic diameters for PEG-b-PLA NPs (dark grey), N₃-NPs, 30% CpG-B NPs and 30% CpG-C NPs for three independent experiments measured via DLS (n=3) in PBS. All results are given as mean t s.d, and p values were determined by a two tailed t-test.

FIGS. 32A-32B: ¹H NMR spectra of the synthesized polymers. Spectra of the PEG-b-PLA (FIG. 32A) and the Azide-PEG-b-PLA (FIG. 32B) polymers in CDCl₃ (600) MHz).

FIG. 33: ¹³C NMR spectra of the Azide-PEG-b-PLA polymer in DMSO-d6 (150 MHz).

FIG. 34: GPC traces of the synthesized PEG-b-PLA and N₃-PEG-b-PLA polymers. Co-elution of the two peaks demonstrates similar molecular weights.

FIG. 35: Gel electrophoresis of CpG-B NPs and CpG-C NPs. Gel electrophoresis after purification of the CpG NPs demonstrates complete removal of of free CpG from the NPs suspension. Free CpG runs through the gel and confirms the 20 base pair length of the CpG. Unpurified CpG NPs show presence of both, CpG NPs in the wells (top) and free CpG migrating in the gel. Purified CpG NPs stayed in the well of the agarose gel, consistence with NPs conjugation and purification.

FIG. 36: Zeta potential measurements for CpG-B NPs in PBS. An increase in CpG-B density on the surface of the NPs results in a slight decrease of surface charge.

FIGS. 37A-37D: Representative dynamic light scattering curves for all different valencies of CpG-C NPs. Curves show size distribution before (grey) and after CpG-C conjugation (blue); (FIG. 37A) 10% valency, (FIG. 37B) 20% valency, (FIG. 37C) 30% valency, (FIG. 37D) 50% valency.

FIGS. 38A-38C: In vitro activity of CpG-C functionalized NPs. (FIG. 38A) Incubation of Raw-Blue macrophage cells (APCs) with either free CpG-C or different valencies of CpG-C NPs (10%, 20%, 30%, 50%) induces the activation of NF-kB and AP-1. The magnitude of activation is quantified via calorimetric output using QUANTI-Blue solution. (FIG. 38B) Activation curves across a range of CpG-C concentrations (3.1-29 μg/mL) delivered on CpG-C NPs at different densities to 100,000 Raw-Blue cells. The absorbance at 655 nm corresponds to TLR activation. (FIG. 38C) Log EC₅₀ values for each activation curve were extrapolated from (FIG. 38B) using a “log(agonist) vs response” nonlinear regression curve fit of the dilution curves.

FIGS. 39A-39D: Comparison of the activation curves of soluble CpG from Invivogen and IDT. (FIG. 39A) Dilution curves and Log EC₅₀ values (FIG. 39B) for the activation of soluble CpG-B from IDT and from Invivogen. (FIG. 39C) Dilution curves and Log EC₅₀ values (FIG. 39D) for the activation of soluble CpG-C from IDT and from Invivogen. Concentrations range from 200 μg/mL to 0.012 μg/mL of CpG.

FIGS. 40A-40B: Calibration curves for CpG NP. (FIG. 40A) CpG-B NPs and (FIG. 40B) CpG-C NPs absorbance was measured at λ=280 nm using free CpG and by taking into account the corresponding NPs absorbance at the same wavelength. Calibration curves are used to measure the exact concentration of CpG on the different NPs valencies. Whereby as CpG concentration it is meant the total concentration of CpG conjugated to the NPs in 60 μL of CpG-NPs solution.

FIGS. 41A-41I: Fabrication and characterization of CpG-Polymer-Nanoparticle (PNP) hydrogels. (FIG. 41A) Vaccine loaded CpG PNP hydrogels are formed when aqueous solutions of PEG-b-PLA NPs and polymer solutions of dodecyl-modified hydroxypropylmethylcellulose (HPMC-C₁₂) are mixed together with aqueous solutions of vaccine cargo comprising CpG NPs (adjuvant) and spike protein (antigen). Multivalent strong and dynamic interactions between NPs and polymers form the hydrogel structure. (FIG. 41B) Vaccine cargo are added to the aqueous NPs solution before loading the aqueous and polymer components in two separate syringes (i). Mixing of the two phases is achieved via an elbow mixer (ii) and results in homogeneous hydrogels (iii). Image of a PNP hydrogel flowing through a 21-gauge needle during injection (iv). After injection the hydrogel forms a solid-like depot (v). (FIG. 41C) PNP hydrogels exhibit different rheological properties such as viscoelasticity, plasticity and thixotropy and can be injected through a syringe needle by applying a force F. (FIG. 41D) Frequency dependent oscillatory shear rheology and oscillatory amplitude sweeps (FIG. 41E) for CpG PNP and bare PNP hydrogels indicate solid-like properties, with tan (δ)=G″/G′ smaller than 1 (G′>G″). Oscillatory amplitude sweeps (FIG. 41E) and stress-controlled flow sweeps (FIG. 41F) of the CpG-C PNP gel, show similar dynamic yield stresses of approximately 1000 Pa (FIG. 41G). (FIG. 41H) Example of shear dependent viscosities of the two analyzed hydrogels demonstrate shear thinning and yielding effects. (FIG. 41I) Step-shear measurements over 3 cycles model the shear rates applied during injection of the hydrogels through a syringe needle and demonstrate the gel ability to self-heal. Alternating low shear rates (0.1 l/s), and high shear rates (10.0 l/s, grey color) are imposed for 60 and 30 s respectively.

FIGS. 42A-42E: Diffusivity of the cargo and gel components in the CpG-PNP hydrogel. (FIG. 42A) FRAP microscopy images of the select area to be photobleached (i) before bleaching, (ii) right after the bleaching process and (iii) after complete fluorescent recovery. (FIG. 42B) Representative fluorescence recovery curve over time of the spike protein at a concentration of 0.27 mg per mL of gel. (FIG. 42C) Diffusivities of spike protein in PNP (n=8) hydrogels measured via FRAP and diffusivity of spike in PBS calculated using Stokes-Einstein equation (Eq. (2)). (FIG. 42D) NPs and spike protein diffusivities in the hydrogel are measured via FRAP and are represented normalized by D_{gel}, the polymer matrix diffusivity. Values close to 1 represent diffusivities similar to the polymer matrix and support the assumption that NPs and spike antigen are caught in the hydrogel network. The dotted line shows D_{cargo}/D_{gel}=1 (n=4-8). (FIG. 42E) Representative schematic of the vaccine loaded PNP hydrogel, showing all the components diffuse slowly within the hydrogel network. All the results are given as mean t s.d.

FIGS. 43A-43E: In vivo humoral response to COVID-19 subunit vaccine. (FIG. 43A) Bolus groups were immunized with a priming dose of 10 μg of spike antigen and 20 μg of CpG at day 0 and received a boost injection of the same treatment at day 21. Gel groups were immunized with a single dose of 20 μg of spike antigen and 40 μg of CpG adjuvant at day 0. For the gel groups no boost dose was given. Serum was collected on day 7, 14, 21, 28 and 35. Neutralization assays were conducted on day 21. The bolus injections were administered subcutaneously (s.c) in a total volume of 100 μL per mouse. The gel groups were s.c injected with 150 μL of gel per mouse. IFN-α and TNF-α cytokines, and IgG antibody levels were quantified. All CpG bolus and gel groups were compared to spike-based vaccine formulations adjuvanted with either PEG-b-PLA NPs or with Alum (n=5). (FIG. 43B) Anti-spike total IgG concentration of the CpG-B adjuvanted and CpG-C adjuvanted (FIG. 43) COVID-19 vaccines, determined via endpoint ELISA with CpG being either in soluble form (free CpG), tethered to the NPs (CpG NPs) or tethered to the NPs and encapsulated in the hydrogel (CpG PNP gel) (n=5). (FIG. 43D) Pre-boost (Day 21) spike-pseudotyped viral neutralization assays for the CpG-B adjuvanted and CpG-C adjuvanted (FIG. 43E) vaccines at a serum dilution of 1:50. Both CpG PNP gel vaccines elicit stronger neutralizing antibody responses than the CpG NPs and free CpG vaccines, not allowing any viral entry in the cells. Each point represents the titers from a single animal (n=5), each bar represents the means from the group. All the results are given as mean±s.d.

FIGS. 44A-44B: Analysis of systemic toxicity. (FIG. 44A) ELISA analysis of IFN-α and TNF-α (FIG. 443) serum at 0 h, 3 h, and 24 of CpG-B and CpG-C adjuvanted vaccines with CpG being either in soluble form (free CpG), tethered to the NPs (CpG NPs) or tethered to the NPs and encapsulated in the hydrogel (CpG PNP gel) (n=5).

### DETAILED DESCRIPTION

The present inventors have engineered a modular PEG-PLA nanoparticle platform to overcome the limitations of TLR agonist therapies such as severe systemic toxicity and a narrow therapeutic window. The ability to tune the size of the PEG-PLA nanoparticles described herein allows efficient transport to lymph nodes which further reduces toxicity while enhancing immunostimulatory effects. In certain embodiments, the nanoparticles described herein are up to about 200 nm in diameter (e.g., about 20 to about 200 nm or about 20 to about 100 nm in diameter or about 30 nm, about 40 nm, about 50 nm, about 60 nm, about 70 nm, about 80 nm, about 90 nm, or about 100 nm in diameter), allowing for passive transport to lymph nodes, thus reducing systemic toxicity and enhancing interactions with target antigen presenting cells.

In certain aspects, the present disclosure provides a PEG-PLA nanoparticle that presents TLR agonist moieties on its surface. These nanoparticles are made by synthesizing PEG-PLA from N₃-PEG-OH which was subsequently functionalized with alkyne derivatives of a TLR agonist. Any alkynated moiety can be conjugated to the polymers, and the Examples also describe conjugating mannose to increase opsonization of particles by immune cells. Optimally sized and biodegradable nanoparticles were then generated through nanoprecipitation of a combination of conjugated and nonconjugated PEG-PLA polymers. The TLR agonist or other conjugated moieties are then presented on the nanoparticle surface and are accessible to target receptors. The modular nature of this platform allows changing both the type(s) of molecule(s) presented on the surface and density of molecule presentation. As such, the present disclosure provides potent, non-toxic TLR agonist nanoparticle adjuvants suitable for use in cancer immunotherapies and vaccines.

In exemplary embodiments, the present disclosure provides a PEG-PLA nanoparticle with a TLR agonist attached to its surface having a controlled size (e.g., about 30 nm or about 50 nm), which allows the nanoparticle to be retained in the lymphatic system and kept out of the systemic circulation. This targeting leads to enhanced efficacy against tumors and reduced toxicity in a mouse model. The nanoparticles can be used on their own as anti-cancer therapy or as an adjunct “add on” therapy to any other immunotherapy (including checkpoint antibodies such as PD1, PDL1, CTLA4, and OX40, and/or immunomodulatory molecules such as cytokines and chemokines) to stimulate an immune response in cancer. The nanoparticles can also be used on their own or combined with controlled release hydrogels for vaccines or anti-cancer therapy (e.g., cancer immunotherapy). Non-limiting examples of hydrogels for controlled release of immunomodulatory compounds are described in International Publication No. WO 2020/072495, the disclosure of which is incorporated herein by reference in its entirety. The present disclosure also provides methods of making the nanoparticles described herein.

In other aspects, the present disclosure provides an injectable and self healing polymer nanoparticle hydrogel platform to prolong the co-delivery of vaccine components to the immune system. In certain embodiments, the nanoparticle hydrogel platform provides an enhanced magnitude and duration of germinal center responses in the lymph nodes by creation of a local inflammatory niche with the hydrogel, coupled with sustained release of vaccine cargo. The present disclosure introduces a simple and effective vaccine delivery platform that increases the potency and durability of subunit vaccines.

### EXAMPLES

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. TLR 7/8 Agonist Presenting Nanoparticles Enhance Anti-PD-L1 Cancer Immunotherapy

### Abstract

Cancer immunotherapy can be augmented with toll-like receptor agonists (TLRas), which interact with immune cells to elicit the appropriate downstream immune response. Use of these drugs is limited due to their extreme potency, and lack of pharmacokinetic control, causing systemic toxicity from unregulated systemic cytokine release. Herein, we overcome these shortcomings by generating PEG-PLA nanoparticles (NPs) that present potent TLR7/8 as on their surface. We hypothesize that the pharmacokinetic profile of the NPs minimizes systemic toxicity, localizing the TLR7/8a presentation to the tumor bed and tumor draining lymph nodes. The nanoparticle platform allows precise control of TLR7/8a valency and the resulting surface presentation. In conjunction with anti-programmed death-ligand 1 (anti-PD-L1) checkpoint blockade, peritumoral injection of TLR7/8a NPs slows tumor growth, extends survival, and decreases systemic toxicity in comparison to the free drug in a murine colon adenocarcinoma model. These NPs constitute a modular platform for controlling pharmacokinetics of toxic immune-stimulatory drugs, resulting in increased potency and decreased toxicity.

### Introduction

Cancer immunotherapies, such as therapeutic immune checkpoint antibodies, are being developed for clinical use at increasing rates. The most widely used cancer immunotherapies are antibodies that prevent interactions of CTLA4/(CD80/CD86) and PD1/PD-L1. Though anti-CTLA4 and anti-PD1/PD-L1 therapies have shown great efficacy in some cancers, the overall response rates are highly variable mainly due to tumor cell evasion mechanisms.¹ Supplementing PD-L1 checkpoint blocking antibodies with toll-like receptor agonists (TLRas) and other innate activators like STING agonists has shown great promise towards overcoming resistance mechanisms that cause low response rates.^{2,3,4,5}. TLR7/8a are potent mimics of ssRNA that can elicit powerful immune responses that have been shown to synergize with immune checkpoint therapies.⁶ Unfortunately, the applicability of TLR7/8a in cancer immunotherapy is currently limited to skin cancers and metastatic cancers presenting on the skin, as systemic distribution results in a severe systemic response.^{7,8,9}, Pharmacokinetic control of these compounds is crucial for their translation into the clinic, emphasizing the need for optimized drug delivery approaches.

TLR7/8a primarily activate pathways in dendritic cells (DCs) by mimicking single stranded nucleic acids which are the natural ligands. Activation of TLR7/8 by ssRNA mimics (TLR7/8a) boosts antigen presentation by DCs (and macrophages) through downstream signaling and cytokine production as part of the larger immune response. Synergy with PD-L1 blockade results from co-administration with TLR7/8a because TLR7/8 as aid lymph node- and tumor-resident dendritic cells in priming naïve T-cells towards tumor antigens, resulting in a tumoricidal behavior that can then be prolonged by the addition of PD-L1 blockade.^{10,11} Immunosuppressive tumors experience low levels of T cell priming, which renders PD-L1 checkpoint blocking ineffective. The addition of stimulatory molecules like TLR7/8a can initiate DC activation and kick-start the downstream T-cell response.

Nanocarriers presenting covalently bound TLR7/8a can activate TLR7/8 receptors.¹² This is due to the TLR7/8 presenting its ligand binding site on the endosomal lumen of DCs and macrophages. Immunogenic constructs presenting TLR7/8a are thus possible, where the pharmacokinetic properties are strictly dictated by the carrier, with the construct itself being immunogenic without release of the TLR7/8a. Analogs of Resiquimod (R848) are popular TLR7/8a for these purposes, with several amine-functionalized analogs developed, some of which show higher potency and vastly different PK/PD profiles, with exacerbated toxicity.¹³ Multiple approaches have been effective in modulating the pharmacokinetics of these analogues. Macromolecular constructs presenting TLR7/8a have been explored for (anti-cancer) vaccine development by Lynn et al. They found that the morphology of the constructed nanoparticles had a significant impact on both T cell induction, and antibody production against a co-presented antigen.^{14,15} Nuhn et al. have likewise shown that pH responsive polymeric micelles with conjugated TLR7/8a is effective in eliciting a non-toxic, local immune response that synergizes with PD-L1 antibody antagonists.^{16,17} Although these macromolecular constructs presenting TLRas often have lower in vitro potency compared to unbound TLRas, they have better controlled tissue distribution and pharmacokinetics.

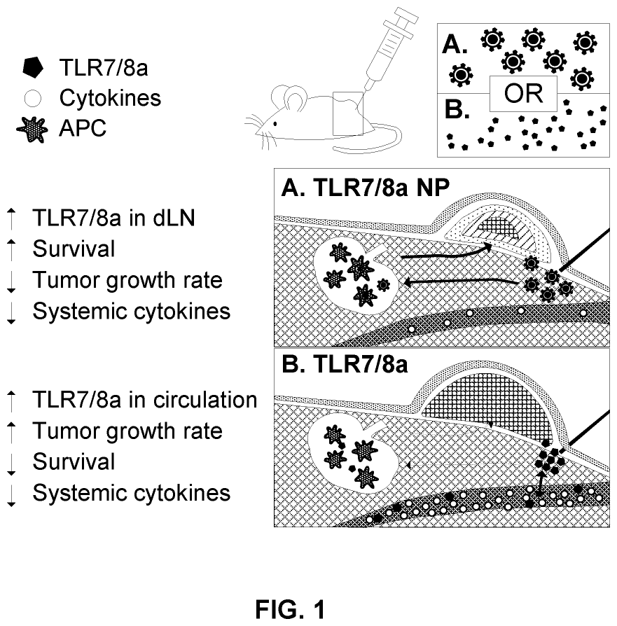

Altering the biodistribution of TLR7/8a also alters the number and biodistribution of different cytokines as well, as it impacts which immune cells that are exposed to TLR7/8 agonism, consequently, modulating the pharmacokinetics results in vastly different pharmacodynamics of the TLRas. Based on these advances, we hypothesize that PEG-PLA NPs can exert similar pharmacokinetic modulation of TLRas, as depicted in FIG. 1. Core-shell nanoparticles, made from PEG-PLA block copolymers, constitutes a modular platform, where the terminus of the PEG corona can present conjugated moieties. Mixing functionalized PEG-PLA and unmodified PEG-PLA at various ratios prior to nanoprecipitation changes the presentation of conjugated targeting moieties and drug densities on the NP surface. PEG-PLA NPs have previously been tested in clinical trials for applications in drug delivery and are considered biocompatible. The PLA core is biodegradable, and the 5 kDa PEG allows for renal clearance, enabling ready elimination from the body and preventing bioaccumulation after treatment. As such, PEG-PLA NPs benefit from the advantages of macromolecules, while functioning as a tunable platform that utilizes materials already tested in clinic.

### Results and Discussion

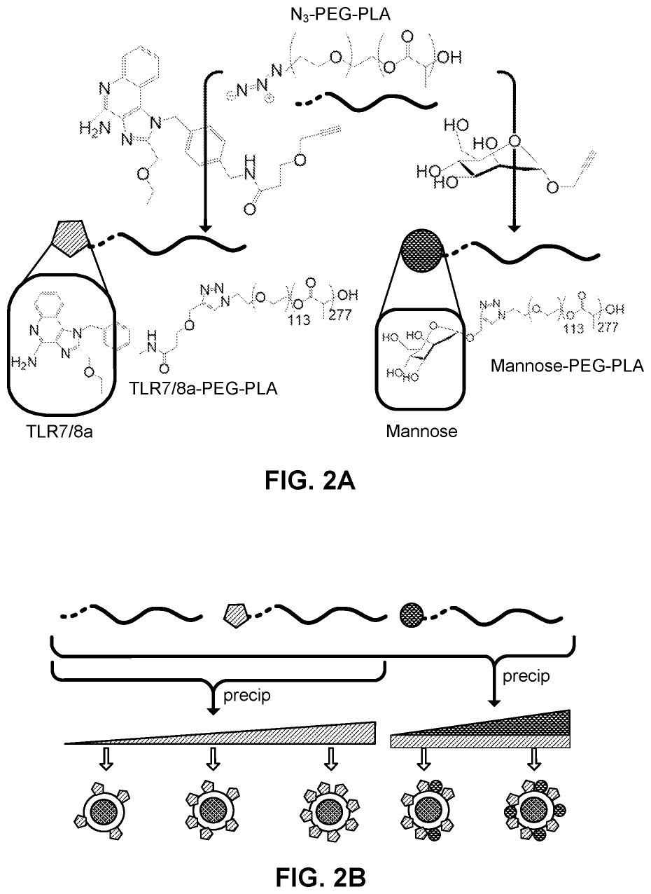

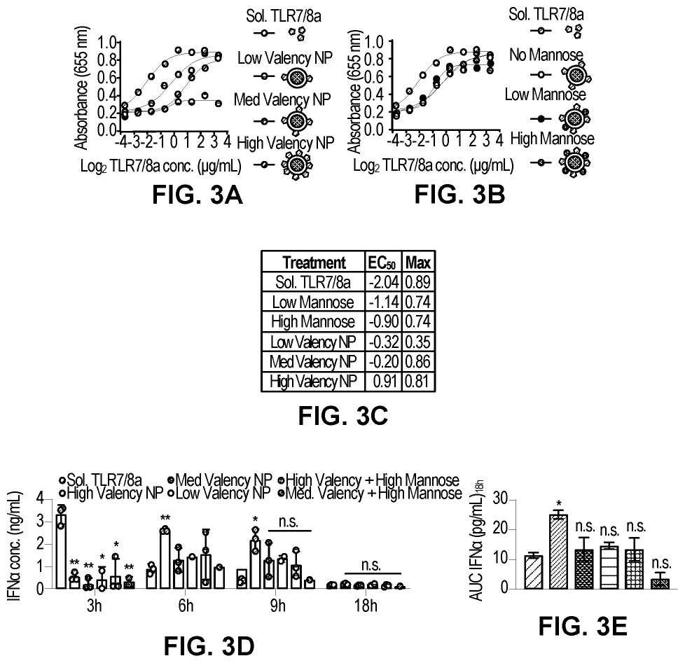

As the basis of a modular PEG-PLA NP platform, we synthesized azide terminated PEG-PLA from N₃-PEG-OH. The polymers were subsequently functionalized with alkyne derivatives of a TLR7/8a or mannose (FIG. 2A). In vitro studies were conducted to optimize TLR7/8a delivery on PEG-PLA NPs. Specifically, the density of TLR7/8a on the NP surface, and the presence of mannose (Table 1), were tested since these variables have been shown to impact NP uptake and receptor activation (FIG. 2B).^{15,18} To assess the ability of TLR7/8a-tethered NPs to activate innate immune cells, we used RAW-Blue mouse macrophage reporter cells (Invivogen). Cells were incubated with TLR7/8a at a range of concentrations (0.08 μg/mL to 10 μg/mL) either in soluble form or tethered to PEG-PLA NPs (TLR7/8a NPs) at different densities to generate concentration-dependent activation curves. NPs were consistent sizes and had similar zeta potentials regardless of the molecule(s) attached to the surface and their density (Tables 2 and 3). The density of TLR7/8a on NPs influenced the EC₅₀ and maximum values of the activation curves (FIG. 3A. FIG. 3C) which are indicators of agonist potency. A lower EC₅₀ is optimal because it indicates that a lower TLR7/8a concentration is needed to reach the half-maximum activation. TLR7/8a presented at a medium or low density on NPs (medium/low valency) resulted in EC₅₀ values that were between 3 and 4-fold greater than EC₅₀ for the soluble TLR7/8a curve (FIG. 3A, FIG. 3C). Unlike low valency NPs which had a very low maximum activation, medium and high valency NPs had similar maximum activation values to that of soluble TLR7/8a (FIG. 3A, FIG. 3C).

TABLE 1
Composition of different nanoparticles tested

| | PEG- | R848-PEG- | Mannose- |
|---|---|---|---|
| NP Treatment | PLA (%) | PLA (%) | PEGPLA (%) |
| High Valency NP | 50 | 50 | |
| Medium Valency NP | 75 | 25 | |
| Low Valency NP | 90 | 10 | |
| High Mannose | 30 | 20 | 50 |
| Low Mannose | 60 | 20 | 20 |
| High Valency/High | | 50 | 50 |
| Mannose | | | |
| Medium Valency/High | 25 | 25 | 50 |
| Mannose | | | |

TABLE 2
NP size by composition and precipitation solvent

| | Diameter | | | |
|---|---|---|---|---|
| | (nm) | S | PDI | S |
| Ratio TLR7/8a- | | | | |
| PEGPLA/PEGPLA | | | | |
| 50/50 | 39.2 | 0.4 | 1.51E−01 | 4.43E−02 |
| 25/75 | 34.9 | 0.4 | 7.81E−02 | 2.81E−02 |
| 10/90 | 33.1 | 0.4 | 8.62E−02 | 1.90E−02 |
| 0/100 | 31.1 | 0.3 | 5.46E−02 | 1.52E−01 |
| 100% AcCN | | | | |
| Ratio TLR7/8a- | | | | |
| PEGPLA/PEGPLA | | | | |
| 50/50 | 38.9 | 0.3 | 1.43E−01 | 2.96E−02 |
| 25/75 | 34.8 | 0.4 | 8.80E−02 | 4.08E−02 |
| 10/90 | 32.7 | 0.3 | 6.97E−02 | 3.50E−02 |
| 1:2 DMSO:AcCN | | | | |
| NP type | | | | |
| High Mannose | 38.6 | 0.3 | 8.47E−02 | 2.19E−02 |
| Med Mannose | 51.5 | 0.4 | 1.64E−01 | 2.34E−02 |
| Low Mannose | 43.5 | 0.2 | 9.27E−02 | 3.23E−04 |

TABLE 3
NP size and PDI by DLS

| Ratio TLR7/8a- | Diameter | |
|---|---|---|
| PEGPLA/PEGPLA | (nm) | PDI |
| 50/50 | 39.5 | 1.36E−01 |
| 25/75 | 33.7 | 1.07E−01 |
| 10/90 | 31.7 | 5.24E−02 |
| 0/100 | 30.3 | 8.56E−02 |
| | Zeta | |
| Ratio TLR7/8a- | Potential | |
| PEGPLA/PEGPLA | (mV) | Std. Dev. |
| 10/90 NP | −14.8 | 0.8 |
| 50/50 NP | −14.2 | 0.6 |
| 50/50 NP (Mannose- | −9.8 | 0.4 |
| PEGPLA) | | |
| MeOPEGPLA NP | −28 | 1 |

TABLE 4
MFI Luminex values and corresponding P-values

| | | | | | | | | FDR | Bonferroni |
|---|---|---|---|---|---|---|---|---|---|
| | | | | | | | | Correction^{#} | Correction^{&} |
| Cytokine | NP 1 | NP 2 | NP 3 | Sol. 1 | Sol. 2 | Sol. 3 | p-value | (q-value) | (p-value) |
| TGFβ | 1119 | 516.5 | 657.75 | 1548.25 | 1063.75 | 945.5 | 0.1791 | 0.8737 | >0.9999 |
| IL10 | 51 | 49.5 | 48.5 | 79.5 | 116.5 | 84.75 | 0.0645 | 0.8737 | >0.9999 |
| VEGF | 79 | 60.75 | 68.5 | 86.25 | 96.75 | 91.25 | 0.0226 | 0.8737 | >0.9999 |
| LIF | 38.25 | 40.75 | 49.75 | 54.75 | 54.25 | 72.5 | 0.0645 | 0.8737 | >0.9999 |
| IL6 | 93.25 | 182.25 | 110.5 | 1548.5 | 2797 | 1463 | 0.0139 | 0.0144 | 0.0984 |
| IFNα | 67.25 | 43.75 | 56.75 | 1287.25 | 2719.75 | 770.5 | 0.0579 | 0.0480 | 0.3817 |
| IL9 | 57 | 42.5 | 59.75 | 85.75 | 78.75 | 406 | 0.2734 | 0.8737 | >0.9999 |
| IL12P70 | 58 | 58 | 58.75 | 16 4.5 | 244.5 | 160.25 | 0.0087 | 0.8737 | >0.9999 |
| IL15/IL15R | 38 | 44.5 | 36.5 | 92 | 136 | 92 | 0.0108 | 0.8737 | >0.9999 |
| IFNα | 39 | 47 | 37.25 | 76.5 | 100 | 77.5 | 0.0061 | 0.8737 | >0.9999 |
| GSCF/CSF3 | 72.25 | 63 | 46 | 76.5 | 116.25 | 87.5 | 0.0818 | 0.8737 | >0.9999 |
| IL22 | 37.25 | 33 | 30.25 | 56.75 | 75 | 60 | 0.0070 | 0.8737 | >0.9999 |
| GMCSF | 29.75 | 33 | 31.5 | 64.75 | 99 | 71 | 0.0114 | 0.8737 | >0.9999 |
| IL27 | 28.5 | 32 25 | 28 | 34.5 | 40.75 | 514.75 | 0.3527 | 0.8737 | >0.9999 |
| IL3 | 27.25 | 34.5 | 30.5 | 30 | 39 | 38.5 | 0.2303 | 0.8737 | >0.9999 |
| IL4 | 38.5 | 44 | 42 | 55 | 66.75 | 59.75 | 0.0073 | 0.8737 | >0.9999 |
| MCSF | 45.5 | 53 | 48 | 56.2.5 | 55.5 | 66.25 | 0.0629 | 0.8737 | >0.9999 |
| IL5 | 98 | 107.75 | 53.75 | 204.25 | 232.75 | 198.25 | 0.0032 | 0.8737 | >0.9999 |
| IL2 | 187.5 | 261.25 | 291.75 | 178.75 | 284.75 | 188.75 | 0.5558 | 0.8737 | >0.9999 |
| MIP1B | 367.25 | 623.75 | 131.25 | 11759.25 | 16535.75 | 8211.25 | 0.0081 | <0.0001 | <0.0001 |
| MCP3 | 2518 | 2288.5 | 3956 | 12351.75 | 12621.25 | 11008.25 | 0.0002 | <0.0001 | <0.0001 |
| IP10 | 974 | 1001.5 | 830.5 | 9204.5 | 8625.25 | 7510 | 0.0001 | <0.0001 | <0.0001 |
| MCP1 | 138.25 | 453.5 | 134.5 | 8219.25 | 13884.5 | 7932 | 0.0073 | <0.0001 | <0.0001 |
| GROA | 127.5 | 511.5 | 662.25 | 3052 | 2490.5 | 1847.75 | 0.0061 | 0.0051 | 0.0287 |
| EOTAXIN | 3291.5 | 1456 | 5327.75 | 5348.5 | 3325.5 | 4960.75 | 0.4059 | 0.1898 | >0.9999 |
| MIPIA | 87.5 | 132.5 | 85.25 | 798 | 1423.25 | 629 | 0.0248 | 0.4998 | >0.9999 |
| RANTES | 309.5 | 261.5 | 307.25 | 1244 | 1706.25 | 898.75 | 0.0134 | 0.3478 | >0.9999 |
| MIP2 | 120.5 | 107.75 | 113.5 | 150 | 141.5 | 132.25 | 0.0124 | 0.8737 | >0.9999 |
| LIX | 240 | 272.25 | 407.5 | 773.75 | 612.5 | 802.25 | 0.0057 | 0.8737 | >0.9999 |
| TNFα | 71 | 150.5 | 79.5 | 738.5 | 1005.75 | 469 | 0.0153 | 0.8.370 | >0.9999 |
| IL18 | 47.75 | 67 | 50.75 | 189.5 | 30.3 | 205.75 | 0.0078 | 0.8737 | >0.9999 |
| IL17A | 27.75 | 35 | 24.5 | 158.25 | 254.25 | 116 | 0.0231 | 0.8737 | >0.9999 |
| IL13 | 46 | 47.75 | 51.25 | 107.5 | 111.5 | 91 | 0.0010 | 0.8737 | >0.9999 |
| IL31 | 35 | 49.5 | 38.25 | 191 | 309.5 | 168 | 0.0145 | 0.8737 | >0.9999 |
| IL1A | 45 | 48 | 44.75 | 65.5 | ?7 | 64 | 0.0057 | 0.8737 | >0.9999 |
| IL23 | 38.5 | 35 | 33.5 | 48 | 52.5 | 52 | 0.0018 | 0.8737 | >0.9999 |
| IL1B | 25.5 | 24.25 | 21.25 | 35.5 | 42.5 | 39.75 | 0.0029 | 0.8737 | >0.9999 |

^{#}Two-stage step-up method of Benjamini, Krieger and Yekutieli on Prism
^{&}Multiple comparisons with Bonferroni method on Prism

These results demonstrate that tethering TLR7/8a to NPs slightly decreases the potency of the molecule in vitro. Previously tested TLR7/8a NP delivery systems typically lead to decreased activation in cell assays compared to the soluble form even if NP delivery is more potent in vivo.¹⁶ In this case the difference in potency and maximum activation across densities may be due to the pattern of TLR7/8a presentation which can lead to receptor clustering that is necessary for the downstream response.²⁰

Recent studies of TLR7/8a delivery have shown that mannose can increase NP recognition and internalization by mannose-binding C-type lectins.¹⁸ A low and high mannose dose were incorporated into the medium valency TLR7/8a NPs and the same RAW-Blue assay was conducted to quantify TLR activation. We found that the presence of mannose shifted EC₅₀ values increasing activation by approximately 2-fold for the low mannose group and by slightly less for the high mannose group. Both mannose doses, however, led to a modest decrease in maximum activation compared to the soluble TLR7/8a group (FIG. 3B, FIG. 3C).

In vitro cell assays do not take into account biodistribution and uptake by a broad array of cells within an organism, so it is important to screen TLR7/8a density and mannose presentation in vivo. To determine in vivo immune activation, serum IFNα was quantified over time following intraperitoneal (IP) administration of TLR7/8a NPs or soluble TLR7/8a in C57BL/6 mice. IFNα is a critical cytokine produced in response to TLR7/8 activation that contributes to the anti-tumor response.²′ Soluble TLR7/8a treatment led to an early spike (3 hours) in serum IFNα followed by a rapid decrease (FIG. 3D). In contrast, all NP treatments led to peak serum IFNα at 6 hours and levels remained elevated through 9 hours (FIG. 3D). The high valency TLR7/8a NP treatment led to a significant increase in IFNα levels at 6 and 9 hours compared to the soluble treatment and a significant increase in the area under the curve of serum IFNα levels over the 18 hours period (FIG. 3D, FIG. 3E). These in vivo experiments showed that TLR7/8a NP treatment prolonged IFNα serum concentrations following a single administration compared to soluble TLR7/8a treatment. Based on these results, the high valency TLR7/8a NP was identified as the most potent NP candidate and was used going forward as a treatment in a murine colon adenocarcinoma model (MC38) to assess potential synergy between TLR7/8a and the checkpoint antibody against PD-L1 (aPD-L1). The effectiveness of cancer immunotherapy depends on activation of tumor-specific cytotoxic T lymphocytes (CTLs).²² The immune checkpoint blockade antibody aPD-L1 blocks an interaction that inhibits CTL activation therefore improving tumor killing.²³ Unfortunately, response rates of only ˜20% have been reported for this antibody treatment likely due to an insufficient number of activated CTLs.²³ A potent, but safe form of TLR7/8a could effectively promote CTL activation would synergize with aPD-L1 resulting in a better therapy than either component alone.

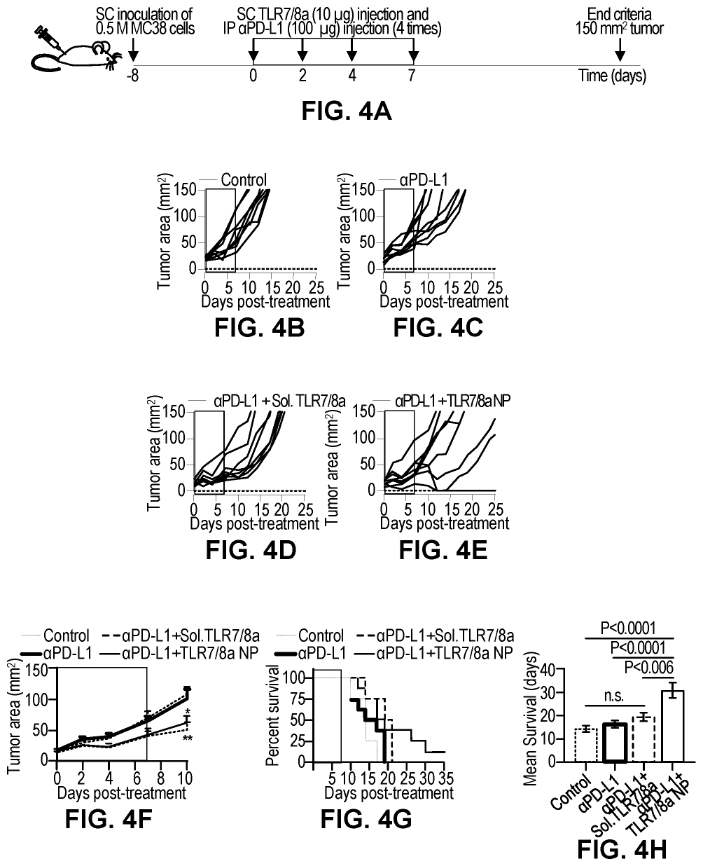

MC38 cells were injected subcutaneously (SC) on the right flank of mice and series of 4 treatment doses began once tumors were measurable (FIG. 4A). Mouse mass and tumor area were monitored until tumors reached the euthanasia criteria of 150 mm² (FIG. 4A). There were 4 treatment groups in this experiment that were as follows: intraperitoneal (IP) phosphate-buffered saline (PBS) injections and peritumoral (PT) PEG-PLA NP (no TLR7/8a), IP aPD-L1 and PT PEG-PLA NP (no TLR7/8a), IP aPD-L1 and PT soluble TLR7/8a, and IP aPD-L1 and PT NP with TLR7/8a. Control mouse tumors grew out consistently and relatively quickly (FIG. 4B). Mice that received aPD-L1 without TLR7/8a had slightly more varied patterns of tumor growth (FIG. 4C). Mice in the cohort that received aPD-L1 and soluble TLR7/8a experienced slowed tumor growth (FIG. 4D). Mice that received aPD-L1 and TLR7/8a NPs had tumor growth curves that varied widely with some tumors shrinking before growing out and others growing out quickly (FIG. 4E). This was the only group in which mouse tumors receded completely during the study (n=2) and one of these mice was ultimately cured (FIG. 4E). The cured mouse was re-challenged with MC38 cells 50 days after the start of the initial treatments and did not re-grow a tumor suggesting immune memory was generated against the cancer.

Over this period, mice that received TLR7/8a either in the soluble form or the NP form had tumors that were significantly smaller than control mouse tumors (FIG. 4F). Mice were monitored for 7 weeks following the start of treatment. All control mice reached the euthanasia criteria by day 17 and all aPD-L1 mice and soluble TLR7/8a mice reached the endpoint by day 21 (FIG. 4G). Notably, the 3 longest-term survivors all received the TLR7/8a NPs (FIG. 4G). To test if tumor growth differed between treatments, we used a restricted maximum likelihood (REML) mixed model. The interaction between treatment and time tested whether treatment altered tumor growth over time. Overall, treatment had an effect on tumor area over time (F_{3,230.2}=30.08, P<0.0001). In pair-wise comparisons all three treatment groups reduced tumor area over time compared to the no treatment control: aPD-L1 alone (F_{1,95.8}=16.11, P=0.0001), aPD-L1+soluble TLR7/8a (F_{1,108.8}=15.73, P=0.0001), and aPD-L1+TLR7/8a NPs (F_{1,115.7}=32.58, P<0.0001). There was no difference in tumor growth rate between aPD-L1 and aPD-L1+soluble TLR7/8a treatment groups. However, when aPD-L1 was delivered together with TLR7/8a NPs, tumor area over time was significantly reduced when compared to treatment with aPD-L1 alone or aPD-L1 with soluble TLR7/8a (F_{1,120.8}=19.06, P<0.0001 and aPDL-1+soluble TLR7/8a (F_{1,133.1}=32.05, P<0.0001).

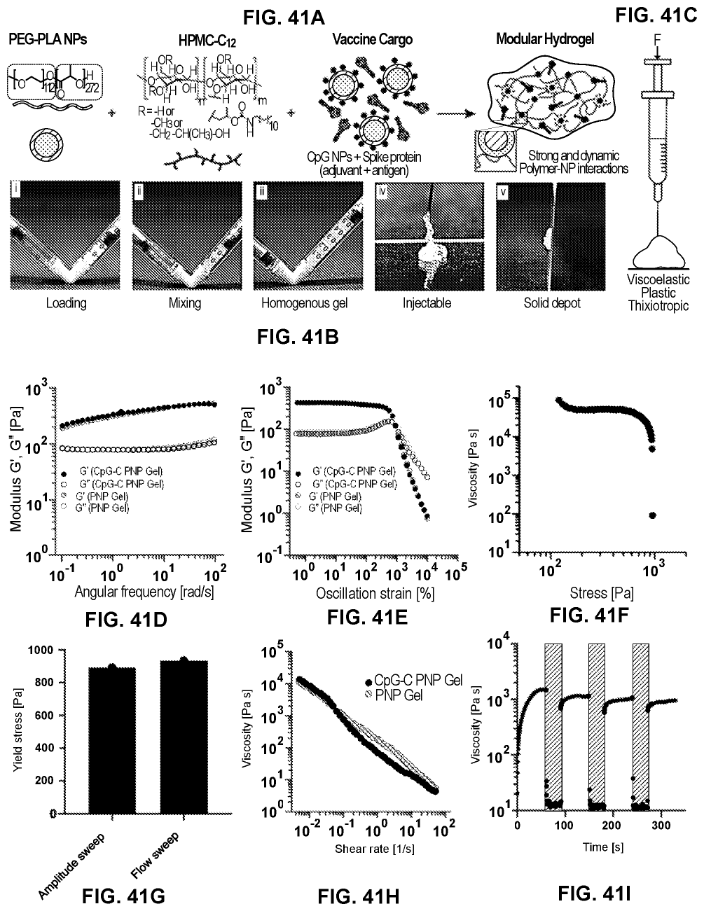

Overall survival was also compared between treatment groups. Neither aPD-L1 nor aPD-L1 with soluble TLR7/8a significantly extended survival compared to the control (FIG. 4H). However, the aPD-L1 with TLR7/8a NP treatment significantly prolonged survival compared to all other treatment groups (FIG. 41I: control, p<0.0001; aPD-L1, p<0.0001; aPD-L1 with soluble TLR7/8a, p=0.006). Together these results reinforce that TLR7/8a synergizes with standard aPD-L1 treatment and that delivery of TLR7/8a on PEG-PLA NPs significantly slows tumor growth and extends survival when compared to the soluble TLR7/8a treatment.

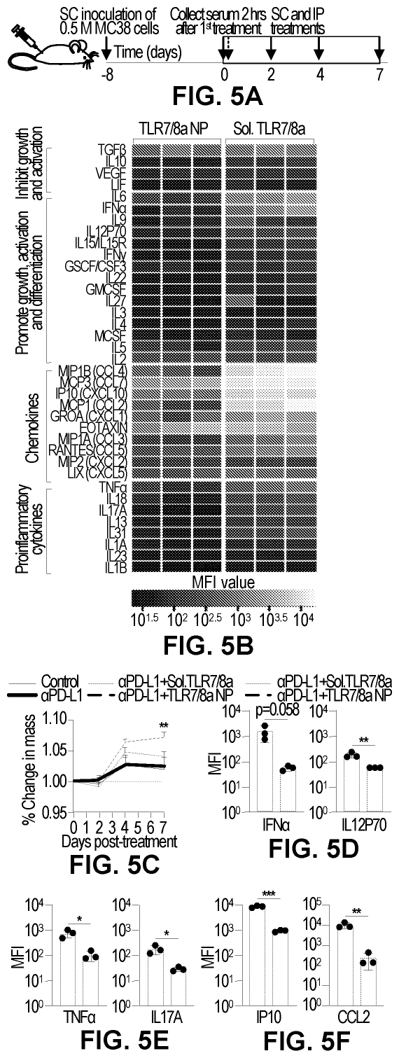

The primary limitation of TLR7/8a therapy is the extreme systemic toxicity.²⁵ The goal of this work was to both increase potency and decrease toxicity of TLR7/8a by tethering it to PEG-PLA NPs to localize its effect. Toxicity was assessed by measuring mouse body mass over the course of treatment and by running Luminex analysis on mouse serum that was collected 2 hours after the initial treatment. On the final treatment day mice that received the TLR7/8a NP treatment had a significantly higher average mass than the control mice (FIG. 5A, FIG. 5B). Severe toxicity and/or illness lead to a decrease in body mass. The mice used in this experiment were 8-weeks-old at the start of treatment and were expected to gain about 5% body mass each week.²⁴ The control mice did not gain as much mass as expected suggesting that the cancer alone is quite toxic (FIG. 5B). The TLR7/8a NP treatment is the least toxic by this measure as it enabled mice to gain a normal percentage of their mass throughout the treatment period (FIG. 5B).

Luminex analysis of serum cytokine levels after the first treatment showed an overall reduction in systemic cytokines in mice that received the TLR7/8a NP treatment as compared to the soluble TLR7/8a treatment (FIG. 5C). Cytokines are key players in the anti-cancer immune response that act by triggering cell differentiation, inhibiting growth, and attracting specific leukocytes to an area of inflammation, among other functions. Type I IFNs, for example, are responsible for priming of tumor-specific CD8 T cells and attraction of NK cells and other leukocytes to the tumor site by promoting production of CXCL9 and CXCL1.²⁶ Unfortunately, high levels of systemic cytokines, including those that are useful and necessary at the proper abundance and location, can cause harmful and dysregulated immune responses. TLR7/8a causes high levels of many cytokines, particularly proinflammatory cytokines which results in systemic immune activation and flu-like symptoms.¹²

We hypothesized that presentation of TLR7/8a on the surface of PEG-PLA NPs would promote draining to lymph nodes and would restrict immune activity to lymph nodes and the tumor environment. TLR7/8a on its own is a small molecule that will rapidly enter circulation while the NPs are in the size regime that has been shown to drain passively to lymph nodes.²⁷ As expected, Luminex results show that mice that received soluble TLR7/8a as compared to TLR7/8a NPs generally had higher levels of serum cytokines across four main classes of cytokines that were quantified: cytokines that inhibit growth and activation; cytokines that promote growth, activation, and differentiation; chemokines; and proinflammatory cytokines (FIG. 5C). Select cytokines that play roles in the anti-cancer response but are extremely toxic at high concentrations were plotted separately as bar graphs. High serum concentrations of the activating cytokines IFNα and IL-12 are associated with autoimmune-effects and flu-like symptoms as observed in a number of pre-clinical and clinical trials.²⁸ As expected, Luminex data showed lower levels of serum IFNα and IL-12 in mice that received the TLR7/8a NPs (FIG. 5D). High levels of proinflammatory cytokines in the serum is a common side effect of R848 treatment.²⁹ Increased serum TNFα and IL17A, for example, are linked general inflammation as well as to sepsis and lupus.³⁰⁻³¹ Mice that received the TLR7/8a NP treatment had a significant reduction in both of these cytokines (FIG. 5E). Chemokines are critical for the function anti-tumor function of TLR7/8a since they can attract various leukocytes to the tumor.²⁶ Unfortunately, when chemokines such as IP10 or CCL2 are at high concentrations in serum, systemic sclerosis occurs.³² Serum concentrations of both IP10 and CCL2 were significantly reduced following TLR7/8a NP treatment as compared to the soluble treatment (FIG. 5F). Overall, delivery of TLR7/8a on PEG-PLA NPs reduced systemic levels of many cytokines that are known to contribute to toxic effects of TLR7/8a.

### Conclusions

TLR7/8 as presented on the surface of PEG-PLA nanoparticles were shown to retain their agonism in vitro, with mannose functionalized particles showing minimal increase in potency. In vivo, the TLR7/8 presenting particles led to prolonged, elevated levels of type I IFN compared to the soluble form. In a murine cancer model, the nanoparticles were shown to effectively synergize with PD-L1 checkpoint blockade to slow tumor growth and extend survival while reducing systemic cytokine release, suggesting lower toxicity. This study supports that macromolecular presentation of TLR7/8 agonists can overcome current toxicity limitations of systemic TLR7/8a delivery and is a viable complement to PD-L 1 checkpoint therapy.

### Methods

Reagents. All chemicals were purchased through Sigma-Millipore, unless stated otherwise. Synthesis of alkynated mannose, alkynated TLR7/8a, and PEGPLA block copolymers is described in the supplementary information.

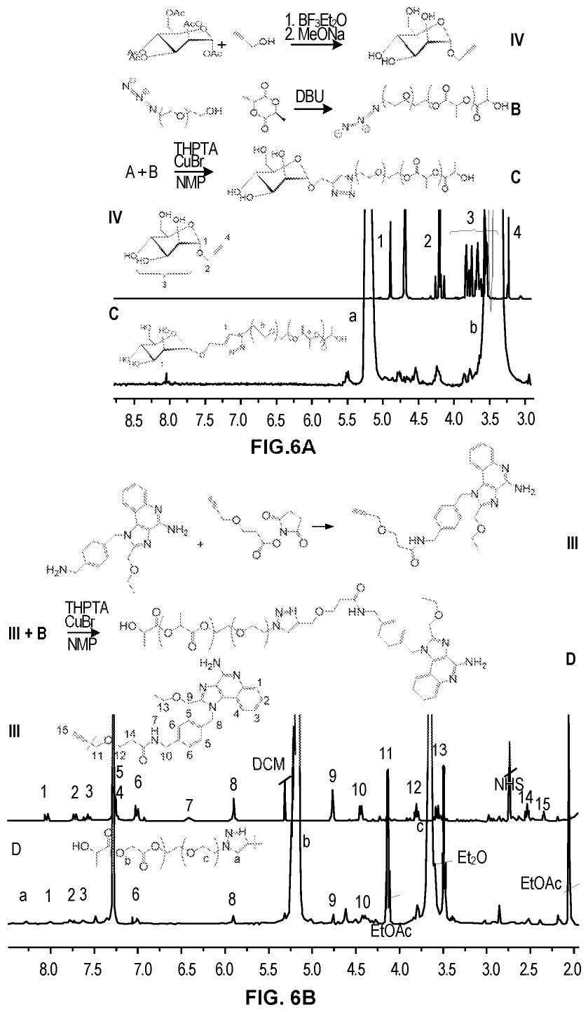

Materials characterization. NMR was obtained using an Inova 300 MHz NMR spectrometer with a Varian Inova console using VNMRJ 4.2 A software. ¹H NMR data for IV, C, III, and D is shown in FIG. 6A-6B. Number-average (Mn) and weight-average (Mw) molar mass and dispersity (Ð=Mw/Mn) of polymers were obtained from gel permeation chromatography (GPC) carried out using a Dionex Ultimate 3000 instrument (including pump, autosampler, and column compartment) outfitted with an ERC Refractomax 520 refractometer. The columns were Jordi Resolve DVB 1000 Å, 5μ, 30 cm×7.8 mm and a Mixed Bed Low, 5μ, 30 cm×7.8 mm, with a Jordi Resolve DVB Guard Column, 1000 Å, 5μ, 30 cm×7.8 mm, 5 cm×7.8 mm. DMF with 10 mM LiBr was used as eluent at 1 mL min⁻¹ at room temperature. Poly (ethylene glycol) were used to calibrate the GPC system. Analyte samples at 2 mg mL⁻¹ were filtered through a nylon membrane with 0.2 μm pore size before injection (20 μL). Data was analyzed using Chromeleon GPC/SEC Software.

Nanoparticle synthesis and characterization. NPs were prepared as previously reported.³³ A 1 mL solution of a combination of PEG-PLA, TLR7/8a-PEG-PLA, and Mannose-PEG-PLA (depending on the experiment as shown in Table 2) in acetonitrile (50 mg/mL) was added dropwise to 10 mL of water under a high stir rate (600 rpm). NPs were purified by ultracentrifugation over a filter (molecular weight cut-off of 10 kDa; Millipore Amicon Ultra-15) followed by resuspension in water to a final concentration of 200 mg/ml. NPs were characterized by dynamic light scattering (DLS) to determine the NP diameters, and zeta potential for the NPs (Tables 3 and 4).

In vitro RAW-Blue reporter assay. The RAW-Blue reporter cell line (InvivoGen, raw-sp) was used in this study. Cells were cultured at 37° C. with 5% CO₂ in Dulbecco's modified Eagle's medium (DMEM; Thermo Fisher Scientific) supplemented with L-glutamine (2 mM), D-glucose (4.5 g/L), 10% heat inactivated fetal bovine serum (Atlanta Biologicals), and penicillin (100 U/mL)/streptomycin (100 μg) and zeocin (100 μg/mL; Invivogen). Serial dilutions of soluble TLR7/8a or one of the TLR7/8a NP formulations (20 μL) was added to a 96-well tissue culture treated plate to final concentrations ranging from 0.08-10 μg/mL. About 100,000 cells were added to each well in 180 μL of media. Cells were cultured for 24 h at 37° C. in a CO₂ incubator before following manufacturer instructions for SEAP quantification (Absorbance at 655 nm). Fits were generated using the “log(agonist) vs. response—Find EC₅₀” in GraphPad Prism with the lower bound constrained to a constant value (0.22) for all fits.

Animal studies. 8-10 week old female C57BL/6 mice were obtained from Charles River and were cared for according to Institutional Animal Care and Use guidelines. Animal studies were performed in accordance with the guidelines for the care and use of laboratory animals; all protocols were approved by the Stanford Institutional Animal Care and Use Committee.

In vivo serum IFNα quantification. Mice were injected with buffer (200 μL) containing soluble TLR7/8a or one of the TLR7/8a NP formulations (25 μg TLR7/8a dose). Mice were injected IP since this administration route resulted in quantifiable cytokine levels across treatment groups. Serum was collected at the indicated times by tail vein blood collection and stored at −80° C. Serum IFNα concentrations were determined by ELISA according to the manufacturer's instructions (PBL Assay Science). Absorbance was measured at 450 nm in a Synergy H1 Microplate Reader (BioTek). Cytokine concentrations were calculated from the standard curves and represented as ng/mL).

MC38 tumor inoculation and treatments. The MC38 colon carcinoma cell line was purchased from Kerafast and cultured using DMEM (Thermo Fisher Scientific) supplemented with L-glutamine (2 mM), 0.1 mM nonessential amino acids, 10% heat inactivated fetal bovine serum (Atlanta Biologicals), penicillin (100 U/mL)/streptomycin (100 μg), and 10 mM Hepes (Sigma-Aldrich). 5×10⁵ MC38 cells suspended in 100 μL of PBS were injected subcutaneously on the right side of the back of C57BL/6 mice. Mice were injected IP on days 8, 10, 12, and 15 post-inoculation with either PBS or 100 μg rat monoclonal anti-mouse aPD-L1 antibody (clone 10F.9G2; Bio X Cell). At the same time as the IP injections, mice were injected subcutaneously (SC) with 50 μL soluble TLR7/8a or high valency TLR7/8a NPs. The soluble TLR7/8a treatment also included PEG-PLA NPs without any conjugated TLR7/8a to account for any effects of the polymer NPs themselves. Tumor growth was monitored by measuring tumors with digital calipers (Mitutoyo Digimatic Caliper) 3 days a week. Tumor area was calculated from a length and width measurement (Area=length×width). Mice were euthanized when tumor volume exceeded 150 mm². On day 50, the tumor-free survivor was re-challenged by SC injection on the right flank with 5×10⁵ MC38 cells and tumor area monitoring continued.

Statistical analysis. Statistical analysis in FIG. 4 and FIG. 6 was done using GraphPad prism software. Data in FIG. 3A-3B were fit using a log(agonist) vs. response fit constrained to 0.22 (average of unconstrained minimum values) for the minimum response value. The EC₅₀ and maximum response values were extrapolated from the fits and reported in FIG. 3C. Mean values in FIG. 3D-3E were compared by ordinary one-way ANOVA with multiple comparisons to the control group (soluble TLR7/8a). In FIG. 5B, mean % change in mass values on day 7 (final day of treatment) were analyzed by t test with multiple comparisons to the control group. In FIG. 5D-5F, mean fluorescence intensity (MFI) values were analyzed by t test. Statistical analysis of MFI values shown on the heatmap in FIG. 5B is shown in Table 4. A t test was run to compare NP treatment to soluble TLR7/8a treatment for each individual cytokine. Additional corrections were done to take into account error from multiple comparisons in the Luminex assay including a false-discovery rate (FDR) Two-stage step-up method of Benjamini, Krieger and Yekutieli correction and Multiple comparisons with Bonferroni method. *P<0.05, **P<0.01, ***P<0.001, n.s.=not significant.

MC38 tumor growth and survival statistical analysis. Mice were assigned randomly to 4 treatment groups (i) no treatment, (ii) aPD-L1, (iii) aPD-L1+soluble TLR7/8a, and (iv) aPD-L1+TLR7/8a NPs. For statistical analysis, tumor area required additional transformation using the natural logarithm to meet the assumptions of homoscedasticity. Analysis was performed in JMP Pro 14. To test if tumor growth differed between treatments, we used a restricted maximum likelihood (REML) mixed model. Mouse was included as a random effect subject. The interaction between treatment and time tested whether treatment altered tumor growth over time. Post-hoc pair-wise comparisons were done between treatment groups and a Bonferroni correction was used to adjust for multiple comparisons (alpha=0.008). P-values for pair-wise comparisons (alpha=0.008): aPD-L1 vs. control, P=0.0001; TLR7/8a vs. control, P=0.0001; NP TLR7/8a vs. control, P<0.0001; a PD-L1 vs. soluble TLR7/8a P=0.7263; NP TLR7/8a vs. a PD-L1, P<0.0001; NP TLR7/8a vs. soluble TLR7/8a, P<0.0001. Survival statistical analysis was performed in SAS Version 9.4. To test if survival differed between treatments, we used a maximum likelihood parametric regression with censored data. Least-squared means were used to compare survival time between individual treatments and Tukey-Kramer post-hoc tests were used to correct for multiple comparison.

Body mass measurements. Mouse body mass was monitored every other day for the first 10 days following the start of treatment using a digital kitchen scale with 0.1 g resolution.

Luminex. 2 hours after the first treatment blood samples were collected by tail vein bleeds. Blood was collected in serum centrifuge tubes (Sarstedt), incubated at RT for 30-60 minutes, and was spun at 10,000 RCF for 5 minutes. Serum was collected and kept frozen at −80° C. until use for Luminex analysis. Mouse 38-plex Procarta kits were purchased from eBiosciences/Affymetrix/Thermo Fisher, Santa Clara, Calif. USA, and used according to the manufacturer's recommendations with modifications as described. Briefly, beads were added to a 96 well plate and washed in a Biotek ELx405 washer. Samples were added to the plate containing the mixed antibody-linked beads and incubated at room temperature for 1 hour followed by overnight incubation at 4° C. with shaking. Cold (4° C.) and room temperature incubation steps were performed on an orbital shaker at 500-600 rpm. Following the overnight incubation, plates were washed in a Biotek ELx405 washer and then biotinylated detection antibody added for 75 minutes at room temperature with shaking. The plate was washed as above, and streptavidin-PE was added. After incubation for 30 minutes at room temperature a wash was performed as above and reading buffer was added to the wells. Each sample was measured in duplicate. Plates were read using a Luminex 200 with a lower bound of 50 beads per sample per cytokine. Custom Assay Chex control beads were purchased from Radix Biosolutions, Georgetown, Tex., and are added to all wells.

Synthesis. The Benzyl amine TLR 7/8 agonist was synthesized as described by Shukla et. al.³⁴, with a modification of the final aromatic substitution and tert butyl carbamate removal on tert-butyl (4-((4-chloro-2-(ethoxymethyl)-1H-imidazo[4,5-c]quinolin-1-yl)methyl)benzyl)carbamate (I), which was done as a one-pot Staudinger-type reaction.³⁵

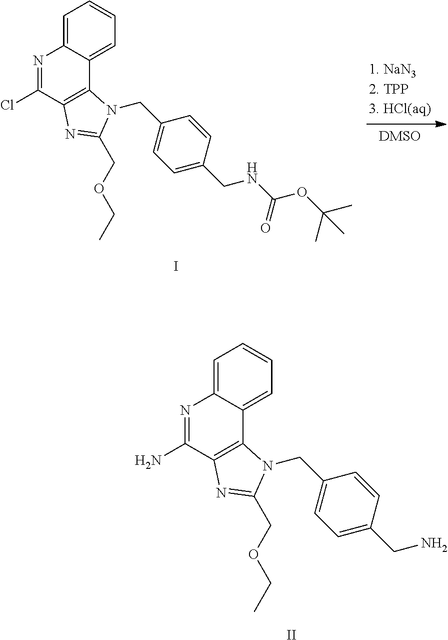

1 and 2 equivalents of NaN₃ (68 mg, 1.04 mmol) was dissolved in 1.5 mL DMSO, and was heated to 90° C. for 2 hours. 2 equivalents of Triphenylphosphine (273 mg, 1.04 mmol) was added, and the temperature was increased to 120° C. and stirred for 16 hours. 0.5 mL 4M HCl (aq) was added and the temperature was lowered to 95° C. for 3 hours. 4 mL of water was added at which a precipitate formed, and the mixture was washed with EtOAc. Na₂CO₃ (sat) was added to the aqueous phase which was extracted with EtOAc, which was dried with with Na₂SO₄ and the solvent removed in vacuo to yield a crude solid. This was purified by silica column chromatography on a biotage system, using a gradient of DCM:MeOH, with 1% trimethylamine in the MeOH. This yielded II (1-(4-(aminomethyl)benzyl)-2-(ethoxymethyl)-1H-imidazo[4,5-c]quinolin-4-amine) (60 mg, 0.16 mmol, 32% yield)

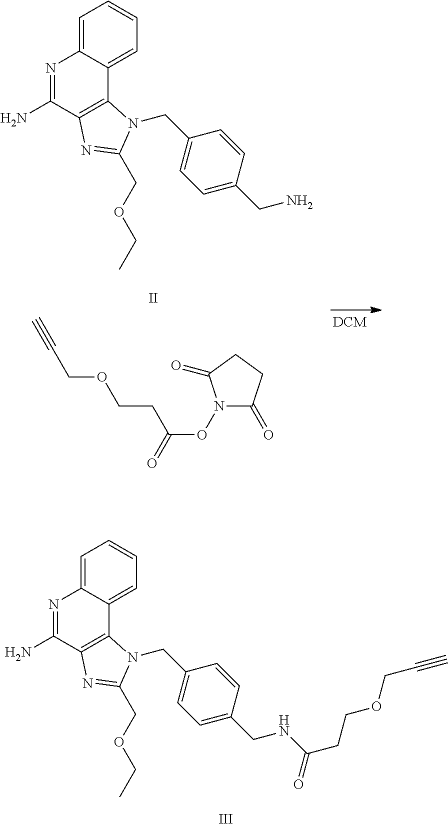

II (25 mg, 69 μmol) and propargyl-N-hydroxysuccinimidyl ester (17 mg, 76 μmol) were dissolved in 1 mL anhydrous DCM, and stirred for 2 hours at room temperature. The reaction mixture was loaded onto a silica column, and the product was purified using a DCM:MeOH gradient. This yielded III (FIG. 6B) N-(4-((4-amino-2-(ethoxymethyl)-1H-imidazo[4,5-c]quinolin-1-yl)methyl)benzyl)-3-(prop-2-yn-1-yloxy)propanamide (25 mg. 53 μmol, 76% yield).

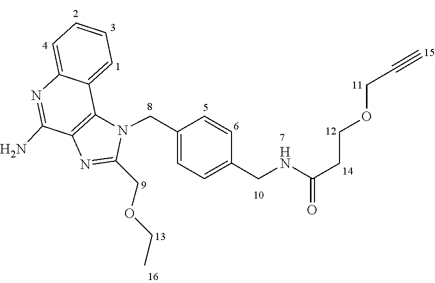 [SMILES: C#CCOCCC(=O)NCc1ccc(Cn2c(COCC)nc3c(N)nc4ccccc4c32)cc1]

¹H NMR (300 MHz, CDCl₃3-d) δ 8.05 (d, J=8.4 Hz, 1H, H1), 7.73 (d, J=8.3 Hz, 1H, H2), 7.58 (t, J=7.9 Hz, 1H, H3), 7.29 (3H, H4, H5), 7.02 (d, J=7.9 Hz, 2H, H6), 6.43 (s, 1H, H7), 5.91 (s, 2H, H8), 4.78 (s, 2, H9), 4.46 (d, J=5.9 Hz, 2H, H10), 4.15 (d, J=2.4 Hz, 2H, H11), 3.82 (t, J=5.7 Hz, 2H, H12), 3.58 (q, J=7.2 Hz, 2H, H13), 2.55 (t, J=5.7 Hz, 2H, H14), 2.36 (t, J=2.4 Hz, 1H, H15), 1.14 (t, J=7.2 Hz, 3H, H16).

¹³C NMR (75 MHz, CDCl₃) δ 171.35, 166.55, 151.77, 150.14, 138.70, 136.66, 136.09, 133.40, 129.84, 128.43, 125.71, 124.66, 121.24, 120.73, 112.79, 79.06, 75.01, 66.77, 66.02, 64.19, 58.44, 58.38, 58.21, 36.88, 31.89, 14.86. HRMS (ESI) calcd. for C₂₇H₂₉N₅O₃+H⁺: 472.2343; found 472.2335.

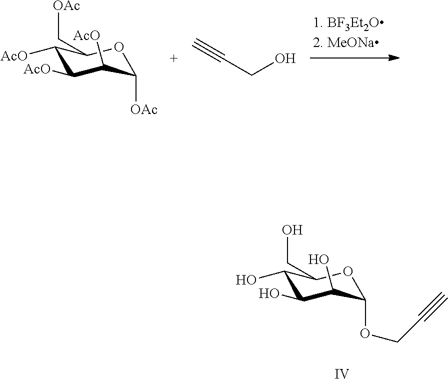

1′-O-propargyl-α-Mannose, IV. Alkynated mannose was synthesized as described elsewhere.³⁶ α-Mannose pentaacetate (1.95 g, 5 mmol) and propargyl alcohol (0.34 g, 0.35 mL, 6.3 mmol) were dissolved in dry DCM (44 mL) while stirring. BF₃Et₂O (9.3 uL, 8 mg, 0.06 mmol) was added at 0° C., and the mixture was stirred for 16 h. The solution was washed with 1M NaOH, and EtOAc was added, and the organic phase was washed with brine and dried over Na₂SO₄. The solution was reduced to approximately 5 mL in vacuo, and diluted with 5 mL hexanes, and purified by silica chromatography with a EtOAc:Hexanes gradient. The product was dissolved in dry MeOH, and 100 μL 2% NaOMe in MeOH was added. The solution was left for one hour, quenched with a drop of acetic acid, and volatiles removed in vacuo to yield IV. ¹H NMR spectrum is shown in FIG. 6A.

N₃-PEG-PLA, MeO-PEG-PLA: PEG-PLA was prepared as previously reported.³⁷ In short, 0.500 g N₃-PEG₍₁₁₄₎-OH or MeO-PEG₍₁₁₄₎-OH in dry DCM (2 mL) with equimolar 1,8-Diazabicyclo[5.4.0]undec-7-ene (15 μL, 15 mg, 0.1 mmol) was rapidly added to a solution of 2.0 g D,L-lactide in 8 mL dry DCM. The mixture was stirred for 8 min, and quenched with the addition of 1 mL acetone with 200 μL acetic acid. The solution was precipitated into 35 mL of a 1:1 mixture of hexanes and diethyl ether in a 50 mL centrifuge tube. The supernatant was discarded, and 35 mL diethyl ether was added to fully precipitate the polymer. This was dissolved in a minimal amount of ethyl acetate, and 35 mL diethyl ether was added to re-precipitate the polymer.

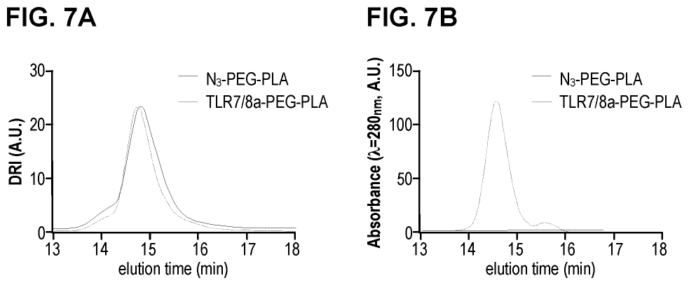

TLR7/8a-PEG-PLA, Mannose-PEG-PLA: Conjugations were performed analogous to the procedure previously reported.³⁷ A 20 mL scintillation vial was charged with III or IV (1.5 eq) and azido poly(ethylene oxide)-b-poly(D,L-lactide) 5 kDa-20 kDa (0.5 g, 20 umol) was dissolved in 4 mL of NMP and sparged with nitrogen for 10 min. 0.1 mL of a degassed CuBr (3.7 mg/mL) and THPTA (16 mg/mL) was added. The reaction mixture was further sparged with nitrogen for 10 min. The reaction mixture is incubated for 16 h at RT, and precipitated into diethyl ether in a 50 mL centrifuge tube to recover the polymer. The polymer was then dissolved in ethyl acetate and precipitated into diethyl ether and dried in vacuo. Synthetic schemes and ¹H NMR data are shown in FIGS. 6A-6B. The conjugation of TLR7/8a was confirmed using SEC as shown in FIGS. 7A-7B.

### REFERENCES

1. Ren, D. X.; Hua, Y Z.; Yu. B. Y.; Ye, X.; He, Z. H.; Li, C. W.; Wang, J.; Mo, Y. Z.; Wei, X. X.; Chen. Y. H.; Zhou, Y. J.; Liao, Q. J.; Wang, H.; Xiang, B.; Zhou, M.; Li, X. L.; Li, G. Y.; Li, Y.; Zeng, Z. Y.; Xiong, W., Predictive biomarkers and mechanisms underlying resistance to PD1/PD-L1 blockade cancer immunotherapy. Mol Cancer 2020, 19 (1).
2. Mullins, S. R.; Vasilakos, J. P.; Deschler, K.; Grigsby, I.; Gillis, P.; John, J.; Elder, M. J.; Swales, J.; Timosenko, E.; Cooper, Z.; Dovedi, S. J.; Leishman, A. J.; Luheshi, N.; Elvecrog, J.; Tilahun, A.; Goodwin, R.; Herbst, R.; Tomai, M. A.; Wilkinson, R. W., Intratumoral immunotherapy with TLR7T8 agonist MEDI9197 modulates the tumor microenvironment leading to enhanced activity when combined with other immunotherapies. Journal for ImmunoTherapy of Cancer 2019, 7 (1), 244.
3. Patel, S. A.; Minn, A. J., Combination Cancer Therapy with Immune Checkpoint Blockade: Mechanisms and Strategies. Immunity 2018, 48 (3), 417-433.
4. Wang, Y.; Su, L.; Morin, M. D.; Jones, B. T.; Mifune, Y.; Shi, H.; Wang, K.-w.; Zhan, X.; Liu, A.; Wang, J.; Li, X.; Tang, M.; Ludwig, S.; Hildebrand, S.; Zhou, K.; Siegwart, D. J.; Moresco, E. M. Y; Zhang, H.; Boger, D. L.; Beutler, B., Adjuvant effect of the novel TLR1/fLR2 agonist Diprovocim synergizes with anti-PD-L1 to eliminate melanoma in mice. Proceedings of the National Academy of Sciences 2018, 115 (37), E8698-E8706.
5. Luo, M.; Wang, H.; Wang, Z.; Cai, H.; Lu, Z.; Li, Y; Du. M.; Huang, G., Wang, C.; Chen, X.; Porembka, M. R.; Lea, J.; Frankel, A. E.; Fu, Y.-X.; Chen, Z. J.; Gao, J., A STING-activating nanovaccine for cancer immunotherapy. Nature Nanotechnology 2017, 12 (7), 648-654.
6. Sato-Kaneko, F.; Yao, S. Y.; Ahmadi, A.; Zhang, S. S.; Hosoya, T.; Kaneda, M. M.; Vamer, J. A.; Pu, M.; Messer, K. S.; Guiducci, C.; Coffman, R. L.; Kitaura, K.; Matsutani, T.; Suzuki, R.; Carson, D. A.; Hayashi. T. Cohen, E. E. W., Combination immunotherapy with TLR agonists and checkpoint inhibitors suppresses head and neck cancer. Jci Insight 2017, 2 (18).
7. Smits, E. L. J. M.; Ponsaerts, P.; Bememan, Z. N.; Van Tendeloo, V. F. I., The Use of TLR7 and TLR8 Ligands for the Enhancement of Cancer Immunotherapy. The Oncologist 2008, 13 (8), 859-875.
8. Pandit, A. S.; Geiger, E. J.; Ariyan, S., Narayan, D.; Choi, J. N., Using topical imiquimod for the management of positive in situ margins after melanoma resection. Cancer Medicine 2015, 4 (4), 507-512.
9. Adams, S.; Kozhaya, L.; Martiniuk, F.; Meng, T. C.; Chiriboga. L.; Liebes, L.; Hochman, T; Shuman, N.; Axelrod, D.; Speyer, J.; Novik, Y.; Tiersten, A.; Goldberg. J. D.; Formenti, S. C.; Bhardwaj, N.; Unutmaz. D.; Demaria, S., Topical TLR7 Agonist Imiquimod Can Induce Immune-Mediated Rejection of Skin Metastases in Patients with Breast Cancer. Clin Cancer Res 2012, 18 (24), 6748-6757.
10. Holcmann. M.; Drobits, B.; Sibilia. M., How imiquimod licenses plasmacytoid dendritic cells to kill tumors. Oncoimmunology 2012, 1 (9), 1661-1663.
11. Aspord, C.; Tramcourt, L.; Leloup, C.; Molens, J. P.; Leccia, M. T.; Charles, J.; Plumas, J., Imiquimod Inhibits Melanoma Development by Promoting pDC Cytotoxic Functions and Impeding Tumor Vascularization. J Invest Dermatol 2014, 134 (10), 2551-2561.
12. Dowling. D. J., Recent Advances in the Discovery and Delivery of TLR7/8 Agonists as Vaccine Adjuvants. ImmunoHorizons 2018, 2 (6), 185-197.
13. Lynn, G. M.; Chytil, P.; Francica, J. R.; Lagová, A.; Kueberuwa, G.; Ishizuka, A. S.; Zaidi, N.; Ramirez-Valdez, R. A.; Blobel, N. J., Baharom, F.; Leal, J.; Wang, A. Q.; Gemer, M. Y. Etrych, T.; Ulbrich, K.; Seymour, L. W; Seder, R. A.; Laga, R., Impact of Polymer-TLR-7/8 Agonist (Adjuvant) Morphology on the Potency and Mechanism of CD8 T Cell Induction. Biomacromolecules 2019, 20 (2), 854-870.
14. Lynn, G. M.; Laga, R.; Darrah, P. A.; Ishizuka. A. S.; Balaci, A. J.; Dulcey, A. E.; Pechar, M., Pola, R.; Gerner, M. Y.; Yamamoto, A.; Buechler, C. R.; Quinn, K. M.; Smelkinson, M. G.; Vanek, O.; Cawood, R.; Hills, T.; Vasalatiy, O.; Kastenmuller, K.; Francica, J. R.; Stutts, L.; Tom, J. K.; Ryu, K. A.; Esser-Kahn, A. P.; Etrych, T.; Fisher, K. D.; Seymour, L. W.; Seder. R. A., In vivo characterization of the physicochemical properties of polymer-linked TLR agonists that enhance vaccine immunogenicity. Nature Biotechnology 2015, 33 (11), 1201-+.
15. Lynn, G. M.; Chytil, P.; Francica, J. R.; Lagova, A.; Kueberuwa, G.; Ishizuka. A. S.; Zaidi, N.; Ramirez-Valdez, R. A.; Blobel, N. J.; Baharom. F.; Leal, J.; Wang, A. Q.; Gemer, M. Y; Etrych, T.; Ulbrich, K.; Seymour, L. W.; Seder, R. A.; Laga, R., Impact of Polymer-TLR-7/8 Agonist (Adjuvant) Morphology on the Potency and Mechanism of CD8 T Cell Induction. Biomacromolecules 2019, 20 (2), 854-870.
16. Nuhn, L.; Van Hoecke, L.; Deswarte, K.; Schepens. B.; Li, Y.; Lambrecht, B. N.; De Koker, S.; David, S. A.; Saelens. X.; De Geest, B. G., Potent anti-viral vaccine adjuvant based on pH-degradable nanogels with covalently linked small molecule imidazoquinoline TLR7/8 agonist. Biomaterials 2018, 178, 643-651.
17. Nuhn. L.; De Koker, S.; Van Lint, S.; Zhong, Z.; Catani, J. P.; Combes, F.; Deswarte, K.; Li, Y.; Lambrecht, B. N.; Lienenklaus, S.; Sanders, N. N.; David, S. A.; Tavemier, J.; De Geest, B. G., Nanoparticle-Conjugate TLR7/8 Agonist Localized Immunotherapy Provokes Safe Antitumoral Responses. Adv Mater 2018, 30 (45), e1803397.
18. Wilson, D. S.; Hirosue, S.; Raczy, M. M.; Bonilla-Ramirez, L.; Jeanbart, L.; Wang, R.; Kwissa, M.; Franetich, J. F.; Broggi, M. A. S.; Diaceri, G.; Quaglia-Thermes, X.; Mazier, D.; Swartz, M. A.; Hubbell, J. A. A.-O. h. o. o., Antigens reversibly conjugated to a polymeric glyco-adjuvant induce protective humoral and cellular immunity. (1476-1122 (Print)).
19. Heil, F.; Ahmad-Nejad, P.; Hemmi, H.; Hochrein, H.; Ampenberger, F.; Gellert, T.; Dietrich, H.; Lipford, G.; Takeda, K.; Akira, S.; Wagner, H.; Bauer, S., The Toll-like receptor 7 (TLR7)-specific stimulus loxoribine uncovers a strong relationship within the TLR7, 8 and 9 subfamily. European Journal of Immunology 2003, 33 (11), 2987-2997.
20. Boehme, K. W.; Compton, T., Innate Sensing of Viruses by Toll-Like Receptors. Journal of Virology 2004, 78 (15), 7867.
21. Zitvogel, L.; Galluzzi, L.; Kepp, O.; Smyth, M. J.; Kroemer, G., Type I interferons in anticancer immunity. Nature Reviews Immunology 2015, 15, 405-414.
22. Sharma. P.; Hu-Lieskovan, S.; Wargo, J. A.; Ribas, A., Primary, Adaptive, and Acquired Resistance to Cancer Immunotherapy. Cell 2017, 168 (4), 707-723.
23. Zou. W.; Wolchok, J. D.; Chen, L., PD-L1 (B7-H1) and PD-1 pathway blockade for cancer therapy: Mechanisms, response biomarkers, and combinations. (1946-6242 (Electronic)).
24. Body Weight Information for C57BL/6J. https://www.jax.org/jax-mice-and-services/strain-data-sheet-pages/body-weight-chart-000664.
25. Kobold, S.; Wiedemann, G.; Rothenfusser, S.; Endres, S., Modes of action of TLR7 agonists in cancer therapy. Immunotherapy 2014, 6 (10), 1085-95.
26. Demaria, O.; Cornen, S.; Daeron, M.; Morel, Y.; Medzhitov, R.; Vivier, E., Harnessing innate immunity in cancer therapy. Nature 2019, 574 (7776), 45-56.
27. Foroozandeh, P.; Aziz, A. A., Insight into Cellular Uptake and Intracellular Trafficking of Nanoparticles. Nanoscale Res Lett 2018, 13 (1), 339.
28. Lasek, W. Zagozdzon, R.; Jakobisiak, M., Interleukin 12: still a promising candidate for tumor immunotherapy? Cancer Immunol Immunother 2014, 63 (5), 419-35.
29. Weeratna, R. D.; Makinen, S. R.; McCluskie, M. J.; Davis, H. L., TLR agonists as vaccine adjuvants: comparison of CpG ODN and Resiquimod (R-848). Vaccine 2005, 23 (45), 5263-5270.
30. Debandt. M.; Vittecoq, O.; Descamps, V.; Le Loët, X.; Meyer, O., Anti-TNF-α-induced systemic lupus syndrome. Clinical Rheumatology 2003, 22 (1), 56-61.
31. Nalbandian, A.; Crispin, J. C.; Tsokos, G. C., Interleukin-17 and systemic lupus erythematosus: current concepts. Clinical & Experimental Immunology 2009, 157 (2), 209-215.
32. Corrado, A., The Th1 chemokine IP-10 in Systemic sclerosis. (1972-6007 (Electronic)).
33. Appel, E. A.; Tibbitt, M. W.; Webber, M. J.; Mattix, B. A.; Veiseh, O.; Langer, R., Self-assembled hydrogels utilizing polymer-nanoparticle interactions. Nature Communications 2015, 6, 9.
34. Shukla, N. M.; Mutz, C. A.; Ukani, R.; Warshakoon, H. J.; Moore, D. S.; and David. S. A., Syntheses of fluorescent imidazoquinoline conjugates as probes of Toll-like receptor 7. Bioorg Med Chem Lett 2010, 20, 6384-6386.
35. Kandalkar, S. R.; Kaduskar, R. D.; Ramaiah, P.; Barawkar, D. A.; Bhuniya, D.; and Deshpande, A. M., Highly efficient one-pot amination of carboxylate-substituted nitrogen-containing heteroaryl chlorides via Staudinger reaction. Tetrahedron Letters 2013, 54, 414-418.
36. Daly. R.; Vaz, G.; Davies, A. M.; Senge, M. O.; and Scanlan, E. M., Synthesis and Biological Evaluation of a Library of Glycoporphyrin Compounds. Chem-Eur J 2012, 18, 14671-14679.
37. Appel, E. A.; Tibbitt, M. W.; Webber, M. J.; Mattix. B. A.; Veiseh. O.; and Langer, R., Self-assembled hydrogels utilizing polymer-nanoparticle interactions. Nature Communications 2015, 6, 9.

### Example 2. Injectable Hydrogels for Sustained Co-Delivery of Subunit Vaccines Enhance Humoral Immunity

### Abstract

Vaccines aim to elicit a robust, yet targeted, immune response. Failure of a vaccine to elicit such a response arises in part from inappropriate temporal control over antigen and adjuvant presentation to the immune system. In this work, we sought to exploit the immune system's natural response to extended pathogen exposure during infection by designing an easily administered slow-delivery vaccine platform. We utilized an injectable and self-healing polymer-nanoparticle (PNP) hydrogel platform to prolong the co-delivery of vaccine components to the immune system. We demonstrated that these hydrogels exhibit unique delivery characteristics whereby physicochemically distinct compounds (such as antigen and adjuvant) could be co-delivered over the course of weeks. When administered in mice, hydrogel-based sustained vaccine exposure enhanced the magnitude, duration, and quality of the humoral immune response compared to standard PBS bolus administration of the same model vaccine. We report that the creation of a local inflammatory niche within the hydrogel, coupled with sustained release of vaccine cargo, enhanced the magnitude and duration of germinal center responses in the lymph nodes. This strengthened germinal center response promoted greater antibody affinity maturation, resulting in a more than 1000-fold increase in antigen-specific antibody affinity in comparison to bolus immunization. Furthermore, our technology improved the efficacy of an influenza hemagglutinin subunit vaccine compared to the most potent adjuvant system used clinically for influenza vaccination, by increasing antibody titers and cross-reactivity against other hemagglutinin variants. In summary, this work introduces a simple and effective vaccine delivery platform that increases the potency and durability of subunit vaccines.

### Introduction

The challenges in designing vaccines towards rapidly mutating pathogens, such as Influenza and HIV, are complex and highly interdisciplinary. To elicit a protective antibody response, vaccines must interact with the appropriate cell types at the right time and place while also providing the necessary cues to guide the immune system¹. With regards to temporal dynamics, natural infections expose the immune system to antigen and inflammatory signals for 1-2 weeks¹. Conversely, the short-term presentation of subunit vaccines from a single bolus administration persists for only 1-2 days¹. The sustained release of soluble antigen provides essential signals to germinal centers (GCs) in the lymph nodes—the structures responsible for the affinity maturation of B cells-leading to high affinity antibody production and humoral immune memory formation²⁻⁴. Recent work demonstrates that slow delivery of an HIV vaccine using osmotic pumps in nonhuman primates resulted in higher neutralizing titers⁵. Similarly, sustained release of a stabilized HIV antigen from a microneedle patch led to increased serum titers and bone marrow plasma cells in mice^{6,7}. It has been reported that the mechanism of traditional adjuvant systems such as aluminum hydroxide (Alum) and MF59 rely on increased antigen persistence in the body⁸⁻¹⁰. These studies, along with others, demonstrate that the kinetics of antigen presentation to the immune system dramatically influences the adaptive immune responses^{5,6,8,9,11-22}. However, these technologies are limited by several factors: (i) the inability to deliver chemically diverse cargo, (ii) restricted time-frame tunability for prolonged release, and/or (iii) low yielding and, oftentimes, challenging multi-step syntheses-. Therefore, there is a significant need for the development of new materials that allow for the sustained exposure of vaccine components to the immune system.

Hydrogel materials are well-suited for drug delivery applications due to their high water content and mechanical tunability that make them similar to many biological tissues²⁴. However, traditional covalently cross-linked hydrogels are often not appropriate for vaccine applications as they cannot be easily administered and have limitations in their delivery kinetics for diverse cargo²⁴. Polymer-nanoparticle (PNP) hydrogels are a type of supramolecular hydrogel where the polymeric constituents are held together by dynamic, multivalent non-covalent interactions between polymers and nanoparticles²⁵⁻²⁸. These materials exhibit many favorable characteristics such as high drug loading capacity, gentle conditions for encapsulation of biologic cargo, sustained delivery of cargo, and mechanical tunability^{24,28,29}. In addition, PNP hydrogels are easily administered due to their shear thinning and self-healing properties^{27,30,31}. The fabrication process, which is easily scaled and therefore highly translatable, involves straightforward mixing of the polymer, nanoparticles (NPs), and an aqueous solution of cargo^{28,32}. This combination of unique properties makes PNP hydrogels ideal candidates for use as a vaccine delivery platform.

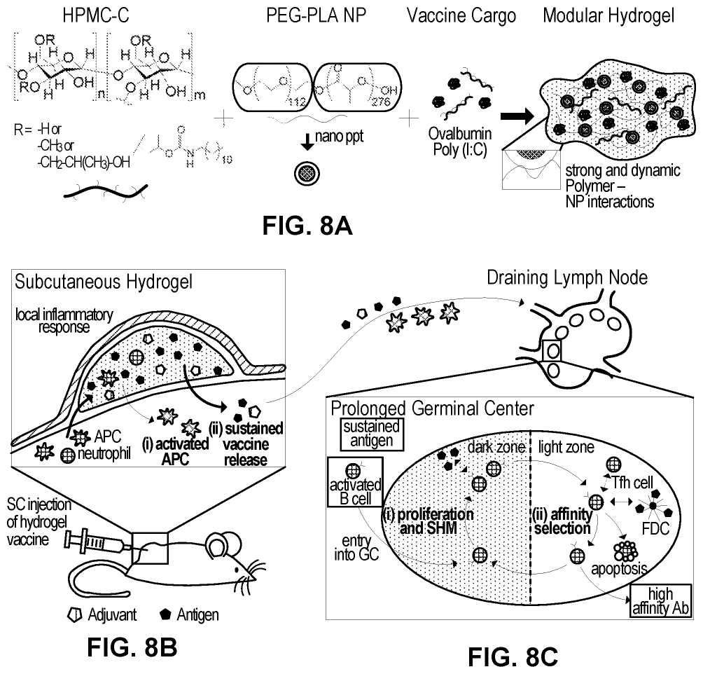

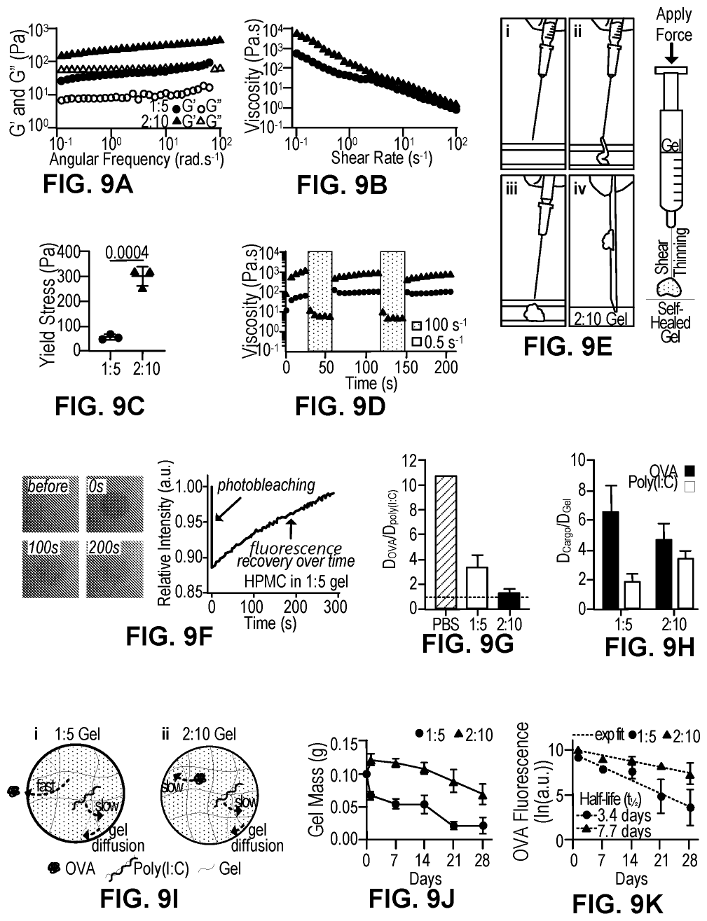

Here, we report the use of PNP hydrogels as a vaccine delivery platform that affords simple encapsulation of vaccine components, provides sustained co-delivery of physicochemically distinct vaccine cargo, and creates a transient inflammatory niche upon administration for activation of the immune system. We show that these materials are easily injected and initiate the vaccine response locally through recruitment of antigen presenting cells (APCs), while also providing sustained release of vaccine cargo (FIGS. 8A-8C). Importantly, our study shows an enhanced and prolonged humoral immune response to a single administration of vaccine-loaded PNP hydrogels, giving rise to average polyclonal antibody affinities equal to those of a monoclonal antibody. Furthermore, we demonstrate the versatility of our delivery platform by showing enhanced potency and durability of antibody responses towards a viral antigen, hemagglutinin (HA) from influenza, compared to clinical adjuvant systems. Overall, this study demonstrates the potential of the injectable PNP hydrogel platform to be used widely for improving subunit vaccine efficacy through simple encapsulation and prolonged delivery of these vaccines.

### Results and Discussion

### Hydrogels for Sustained Vaccine Exposure

We designed a PNP hydrogel material that can load vaccine components with high efficiency, is injectable, and can be tuned to co-deliver subunit vaccine components over prolonged timeframes. Our PNP hydrogels form rapidly when aqueous solutions of hydroxypropyl methylcellulose derivatives (HPMC-C₁₂) are mixed with biodegradable polymeric NPs composed of poly(ethylene glycol)-b-poly(lactic acid) (PEG-PLA) (FIG. 8A, Table 5). Prior to mixing, the two components are solutions, but upon mixing a hydrogel rapidly forms multivalent and dynamic non-covalent interactions between the HPMC polymer and the PEG-PLA nanoparticles creating the physical cross-links that give rise to the hydrogel structure itself. Moreover, as is true with other supramolecular hydrogel platforms, all components that are mixed into the container become part of the self-assembled PNP hydrogel, ensuring 100% encapsulation efficiencies for any cargo. Furthermore, this simple synthesis enables the creation of numerous hydrogel formulations by changing the ratio of HPMC-C₁₂ to NP to an aqueous solution²⁸. We chose two formulations based on their differences in mechanical properties: (i) 1 wt % HPMC-C₁₂+5 wt % NP, designated “1:5”, and (ii) 2 wt % HPMC-C₁₂+10 wt % NP, designated “2:10”. The future translation of our platform is simplified by the ease of synthesis along with the stability of the individual components of our system³³.

TABLE 5
Nanoparticle Characterization (Measured with DLS).

| | Diameter (nm) | Zeta Potential (mV) |
|---|---|---|
| PEG-PLA NP (n = 4) | 32 ± 4 | −28 ± 7 |
| TLR 7/8 PEG-PLA NP(n = 4) | 29 ± 3 | −10 ± 1 |

TABLE 6
Cargo and Polymer Diffusivities (Measured with FRAP).

| Sample | Diffusivity (μm²/s) |
|---|---|
| Poly(I:C) in PBS* | 7.2 |
| OVA in PBS* | 76.6 |
| Poly(I:C) in 1:5 | 1.7 ± 0.3 |
| OVA in 1:5 | 6 ± 1 |
| HPMC—C₁₂ in 1:5 | 0.9 ± 0.1 |
| Poly(I:C) in 2:10 | 2.4 ± 0.1 |
| OVA in 2:10 | 3.2 ± 0.6 |
| HPMC—C₁₂ in 2:10 | 0.68 ± 0.08 |
| Poly(I:C) in PEG Gel** | 0.4 |
| OVA in PEG Gel** | 66.2 |

*Calculated with Stokes-Einstein (details in methods)
**Calculated with Multiscale Dynamic Model (details in methods)

TABLE 7
Flow cytometry antibody information.

| Antibody (all anti-moose) | Manufacturer | Clone |
|---|---|---|
| CD19-PerCP-Cy5.5 | BioLegend | 6D5 |
| GL7-A488 | BioLegend | GL7 |
| CD95-PE-Cy7 | BD Biosciences | Jo2 |
| CXCR4-BV421 | BioLegend | L276F12 |
| CD86-BV785 | Prepared in Pulendran | GL1 |
| IgG1-PE | BD Biosciences | A85-1 |
| CD4-BV650 | BioLegend | GK1.5 |
| CXCR5-BV421 | BioLegend | L138D7 |
| PD1-A647 | BioLegend | 29F.1A12 |
| CD45-BV785 | BioLegend | 30-F11 |
| CD3-BV650 | BioLegend | 17A2 |
| CD19-Alexa700 | BD Biosciences | 1D3 |
| CD11c-APC-Cy7 | ebioscience | N418 |
| Ly6G-BUV395 | BD Biosciences | 1A8 |
| CD11b-BV650 | BioLegend | M1/70 |
| MHCII-BV510 | BioLegend | M5/114.15.2 |

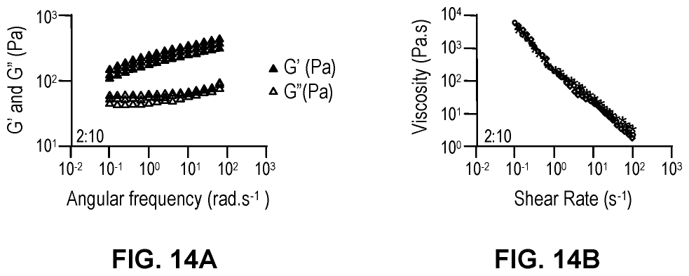

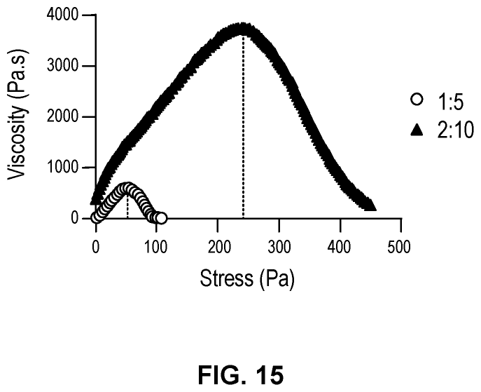

The rheological properties of the hydrogel formulations of interest were then measured. Frequency-dependent oscillatory shear experiments, performed in the linear viscoelastic regime, demonstrated a formulation-dependent frequency response (FIG. 9A). At a representative angular frequency (ω=10 rad/s), the 1:5 and 2:10 gels exhibited storage moduli (G′) of 50 Pa and 350 Pa, respectively. An angular frequency sweep showed that gels remained solid-like across a wide range of frequencies, with the G′ remaining above the loss modulus (G″) at all frequencies tested (FIG. 9A, FIG. 14). A shear rate sweep showed that the viscosity of these PNP hydrogels decreased over two orders of magnitude with increasing shear rates, thereby demonstrating their ability to shear-thin (FIG. 9B, FIG. 14). The 1:5 gel exhibited a lower viscosity than the 2:10 gel across the shear rates tested: 10⁻¹ to 10² s⁻¹ (FIG. 9B). The yield stress of these materials was determined by a stress ramp to be approximately 60 Pa and 300 Pa for the 1:5 and 2:10 gels, respectively (FIG. 9C, FIG. 15). These values describe both gels' abilities to remain solid-like under low stresses (i.e., before and after injection), therefore maintaining a solid gel structure after injection and preventing flow from the injection site which facilitates the creation of a local inflammatory niche³¹.

Both gel formulations were shown to be injectable, a critical consideration for future translation of our material as a vaccine platform. Injectability was tested by measuring the viscosity of the gels when alternating between a high shear rate (100 s⁻¹) reminiscent of the injection process, and a low shear rate (0.5 s⁻¹) reminiscent of the working conditions upon implantation²⁶. The viscosity of both gel formulations decreased by over two orders of magnitude with the applied high shear rate, and rapidly recovered (<5 s) to their original viscosity when returned to a low shear rate (FIG. 9D and FIG. 9E).

Vaccine Cargo Dynamics

Subunit vaccines are composed of two main components: (i) antigen, which directs the antibody response to a specific substance, and (ii) adjuvant, which enhances the innate immune response³⁴. These biomolecules can have vastly different sizes and physicochemical properties³⁴, providing a challenge for controlled co-delivery because these characteristics typically dictate the diffusivity of these compounds and their release kinetics from hydrogel materials in vitro and in vivo³⁵. To understand how PNP hydrogels can be used to deliver a wide range of cargo, we chose a model vaccine that incorporated an antigen and adjuvant with distinct physicochemical properties and very different molecular weights. We used the protein ovalbumin (OVA; MW=43 kDa) as a model antigen and Poly(I:C) (a toll-like receptor 3 agonist (TLR3); MW>1 MDa)^{36,37} as the adjuvant. Poly(I:C), a double-stranded RNA mimic that activates endosomal TLR3, is a potent type I interferon producer and has been used successfully as an adjuvant in preclinical vaccine studies, though it has not been approved for clinical use to date³⁷⁻³⁹. Importantly, this highly tunable materials platform enables these components to be easily replaced with other antigens and adjuvants for future applications.

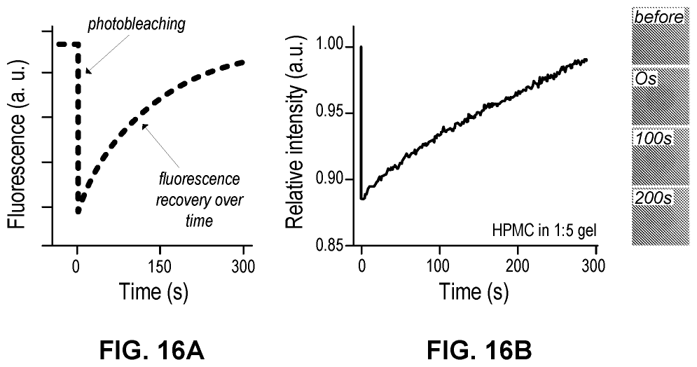

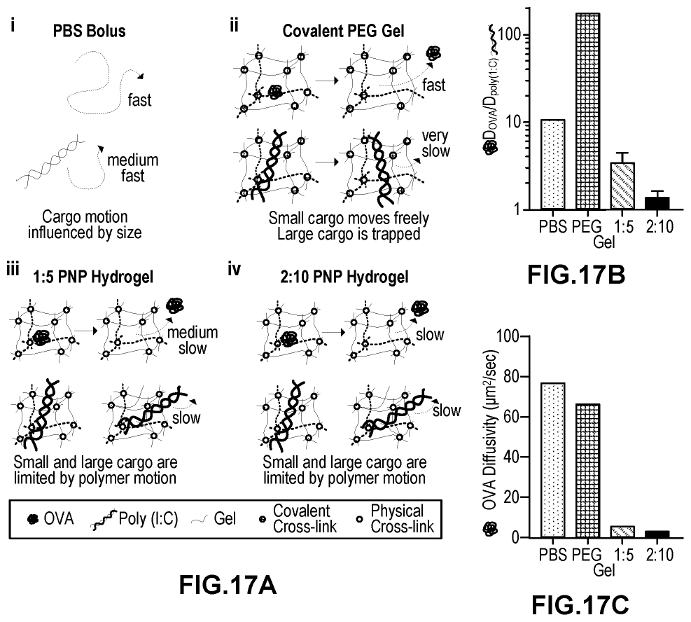

To characterize the dynamics of vaccine exposure expected from the vaccine-loaded gels, we assessed the diffusivities of both vaccine cargo in each of the two gel formulations of interest using fluorescence recovery after photobleaching (FRAP) experiments (FIG. 9F. FIG. 16). The PNP gels are unique compared to traditional covalently cross-linked gels as their bonds are dynamic and continuously rearrange²⁸. This feature results in self-diffusion of the polymer network over time within the intact hydrogel^{40,41}. OVA's hydrodynamic radius (R_{H}) in PBS is reported in the literature to be 3.2 nm⁴², corresponding to a diffusivity value, D, of 76.6 μm²/s. Using multi-angle light scattering (MALS) we measured Poly(I:C)'s hydrodynamic radius to be much larger, with an R_{H} of 34.2 nm, corresponding to a D value of 7.2 μm²/s. This difference in hydrodynamic size results in OVA having more than a 10-fold greater diffusivity than Poly(I:C) in PBS (FIG. 9G) and a 100-fold difference in a typical covalently cross-linked hydrogel (FIG. 17). In contrast, FRAP measurements of the molecular cargo in the PNP gels demonstrated that the hydrogel mesh reduced cargo diffusivities by multiple orders of magnitude, whereby the diffusivity of OVA was determined to be 5.76 μm²/s in the 1:5 gel and 3.2 μm²/s in the 2:10 gel. The measured diffusivities for all experiments can be found in Table 6.

Interestingly, the ratio of OVA to Poly(I:C) diffusivities (D_{OVA}/D_{Poly(I:C)}) was 3.4 in the 1:5 gel, and only 1.4 in the 2:10 gel (FIG. 9G). When comparing the diffusivity of OVA to the self-diffusion of the hydrogel matrix, D_{Gel}, which was determined by tracking the diffusivity of HPMC-C₁₂ within the hydrogel, we saw that D_{OVA}/D_{Gel} was ˜6.5 in the 1:5 gel, but only 4.7 in the 2:10 gel, while the ratio of Poly(I:C) to the hydrogel self-diffusion (D_{Poly(I:C)}/D_{Gel}) was approximately 2 for the 1:5 gel and 3.5 for the 2:10 gel (FIG. 9H). These data suggest that in both gels, Poly(I:C) is immobilized by the hydrogel mesh (i.e., Poly(I:C) is larger than the effective mesh size of the hydrogel; FIG. 17) such that the diffusion of Poly(I:C) in both gels is limited by the self-diffusion of the hydrogel network. The diffusion of OVA, however, is more limited by hydrogel self-diffusion in the 2:10 gel and appears to be small enough to diffuse more freely in the 1:5 gel (FIG. 9I, FIG. 17). Accordingly, the diffusivity of both OVA and Poly(I:C) is restricted by the self-diffusion of the hydrogel network in the 2:10 gel, enabling sustained co-delivery of the two cargo despite their large difference in physicochemical properties.

The in vivo cargo dynamics were characterized by assessing the timeframes of OVA retention in the subcutaneous (SC) space following in vivo administration. The 1:5 and 2:10 gels were loaded with Alexa Fluor 647 conjugated OVA and explanted to measure gel mass and cargo fluorescence at multiple time-points up to 4 weeks after vaccine administration. The 1:5 gels reached half of their initial mass after 2 weeks whereas the 2:10 gels retained half of their initial mass past 4 weeks (FIG. 9J). The cargo fluorescence was measured after explanation and mechanical disruption. From these in vivo data we calculated an OVA retention half-life of 3.4 days and 7.7 days for the 1:5 and 2:10 gels, respectively (FIG. 9K).

### Humoral Immune Response to Vaccination

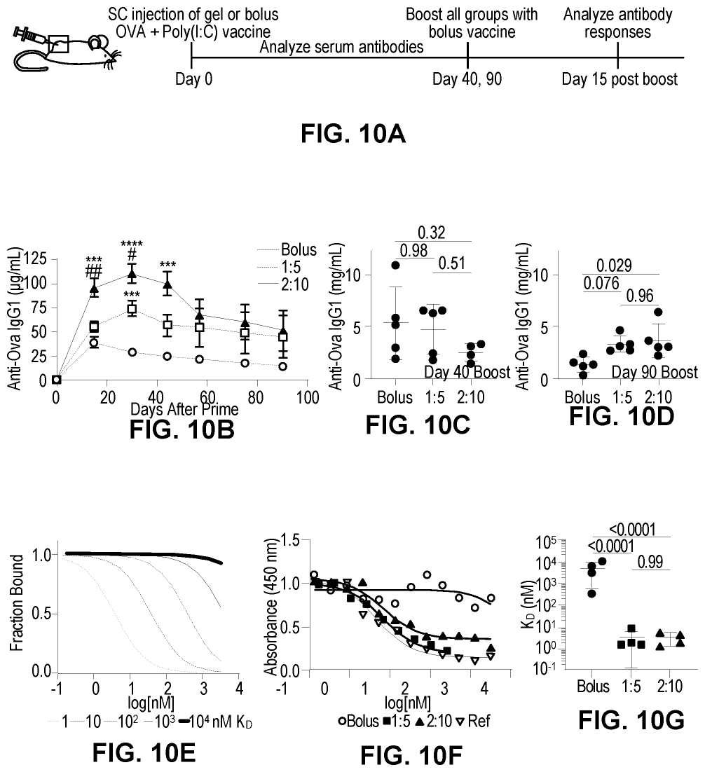

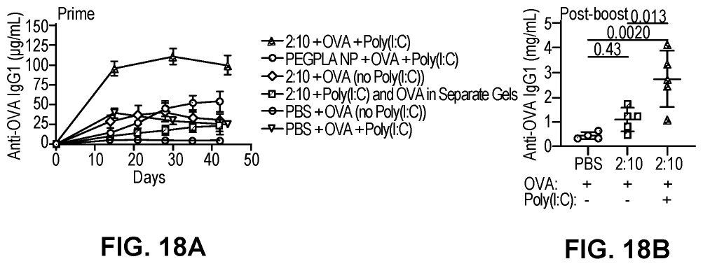

To investigate if sustained vaccine exposure from a single administration of the gel-based vaccines (i.e., gel carrying OVA and Poly(I:C)) could enhance the magnitude and duration of the humoral response, we quantified antigen specific antibodies in the serum after SC injection (FIG. 10A). Humoral immunity is a component of the adaptive immune response mediated by antibodies, which are critical to viral immunity³⁴. The peak concentration of OVA-specific serum antibodies was 2-3 times higher for mice receiving the gel-based vaccines than mice receiving the same vaccine in a standard PBS bolus administration (FIG. 10B). The 1:5 and 2:10 gel-based vaccines yielded antibody concentrations higher than the bolus peak response past day 90 (FIG. 10B). We also observed that the gel alone acts as a mild adjuvant, but the addition of Poly(I:C) significantly improves the potency and durability of the antibody response (FIG. 18). Additionally, delivering antigen and adjuvant in separate gels on contralateral flanks was not as effective as co-delivery of OVA and Poly(I:C) from the same gel (FIG. 18). The increased magnitude and duration of the primary antibody response suggests that delivery of vaccines in PNP hydrogels would afford more durable protection against target pathogens than bolus administration of the same vaccines.

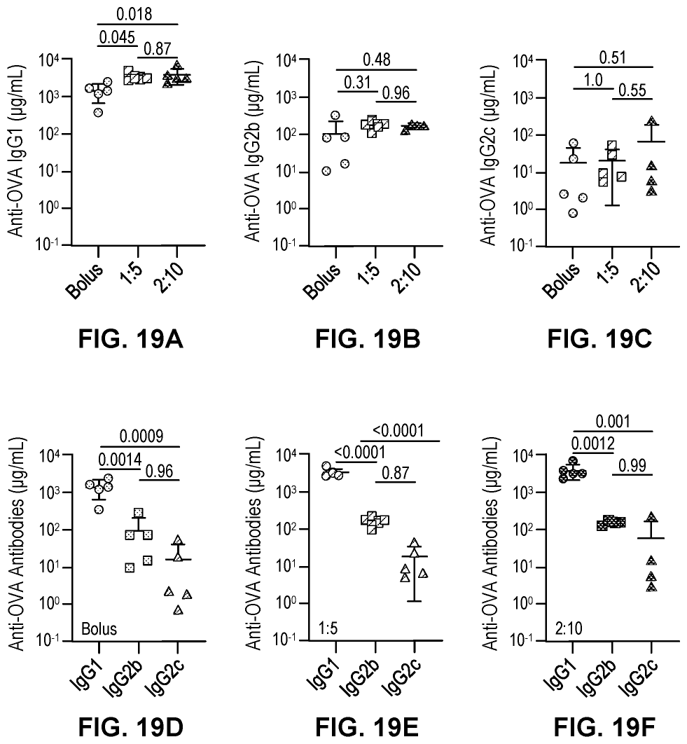

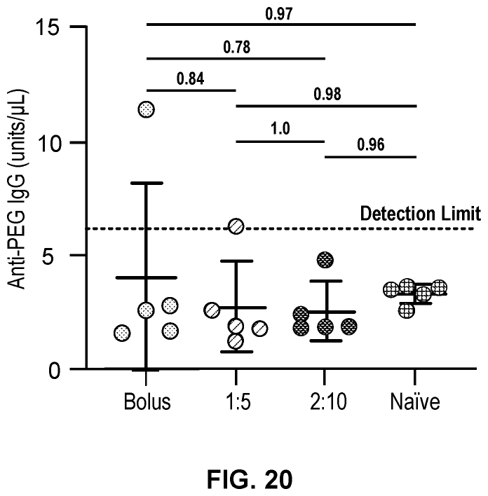

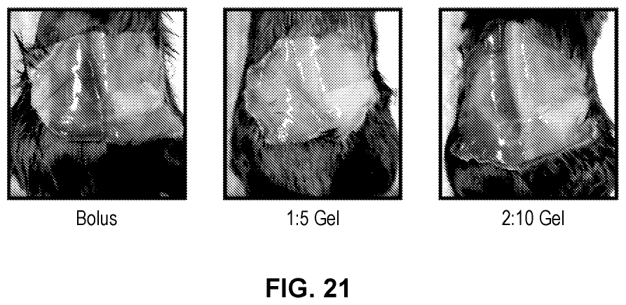

Most vaccines are administered with booster shots to increase the immune memory and ensure long-term protective antibodies are produced³⁴. To study the prime-boost response, we administered OVA and Poly(I:C) in PBS for all groups at 40 or 90 days post-prime in separate mouse cohorts (FIG. 10A). The OVA-specific serum IgG1 antibodies were assessed 15 days after the boost⁴³. The day 40 boost led to statistically equivalent antibody concentrations in all groups (FIG. 10C); however, when the boost was administered 90 days after priming, the mice primed with the 2:10 gel responded with higher antibody titers than the bolus vaccine primed mice (FIG. 10D). Both gel groups exhibited an equivalently strong antibody response to boosts administered at both time-points (FIG. 10C and FIG. 10D), indicating robust humoral memory compared to the bolus group, which had a 3.8-fold decrease in antibody production in response to boosts at 40 days and 90 days (p=0.038). The described antibody responses all refer to IgG1 antibodies, which were the most highly represented among the IgG subclasses. Characterization of IgG2b and IgG2c anti-OVA antibodies can be found in the supplemental information (FIG. 19). Importantly, we see no detectable anti-PEG antibodies developed and no noticeable fibrotic response to these hydrogel-based vaccines (FIG. 20, FIG. 21).

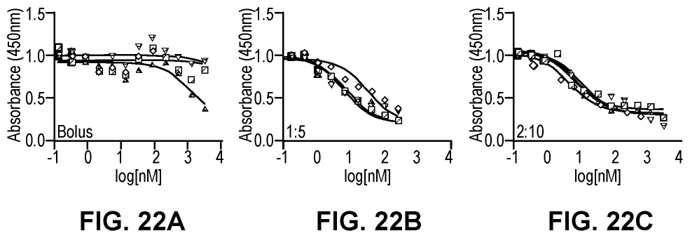

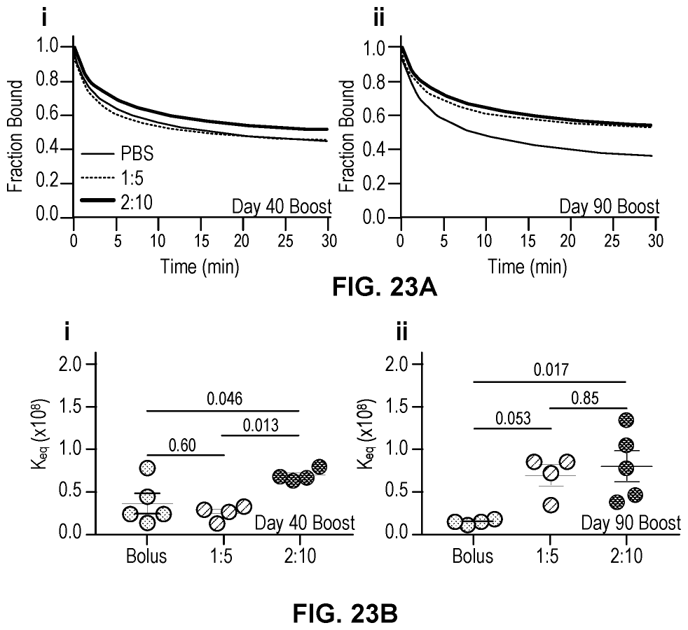

To investigate the quality of the humoral response, we also determined the affinities of the serum antibodies after the 90-day boost towards OVA using a competitive binding enzyme-linked immunosorbent assay (ELISA) (FIGS. 10E-10G). We found that the polyclonal population of antibodies produced by the mice that received the 1:5 or 2:10 gel vaccine prime had K_{D} values of approximately 3 nM, while the antibodies produced by the mice receiving OVA and Poly(I:C) in a PBS bolus prime had a K_{D} value of 3,000 nM (FIGS. 10F-10G, FIG. 22). PNP gel-based vaccination, therefore, results in at least a 1000-fold enhancement in affinity maturation of the antibodies. We validated the trends seen in the competitive binding ELISA with surface plasmon resonance (SPR) experiments (FIG. 23). The increase in antibody affinity observed for the gel groups suggests that the prolonged vaccine exposure with the gel prime led to an enhancement of GC responses and B cell affinity selection⁴.

Local Inflammatory Niche within the Hydrogel Depot

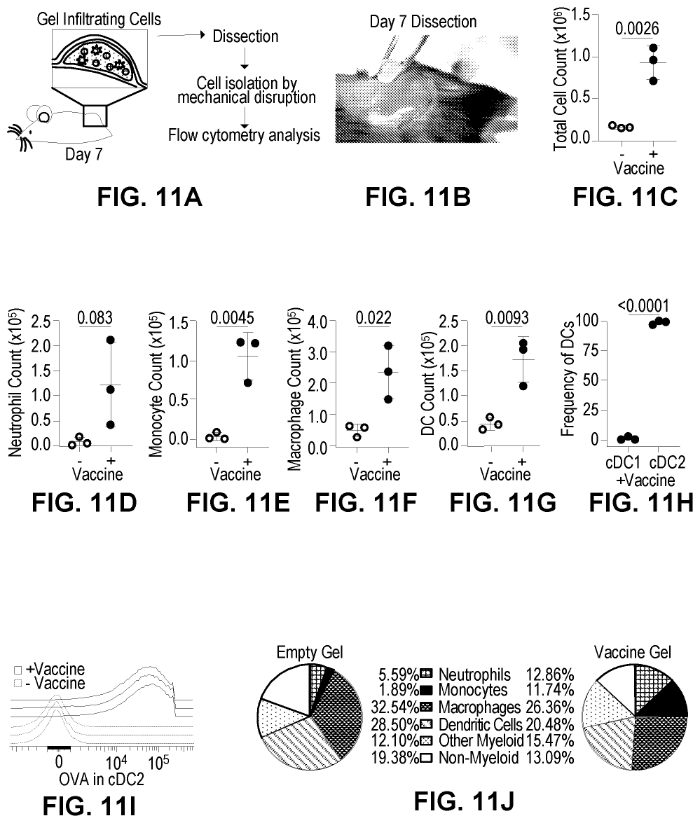

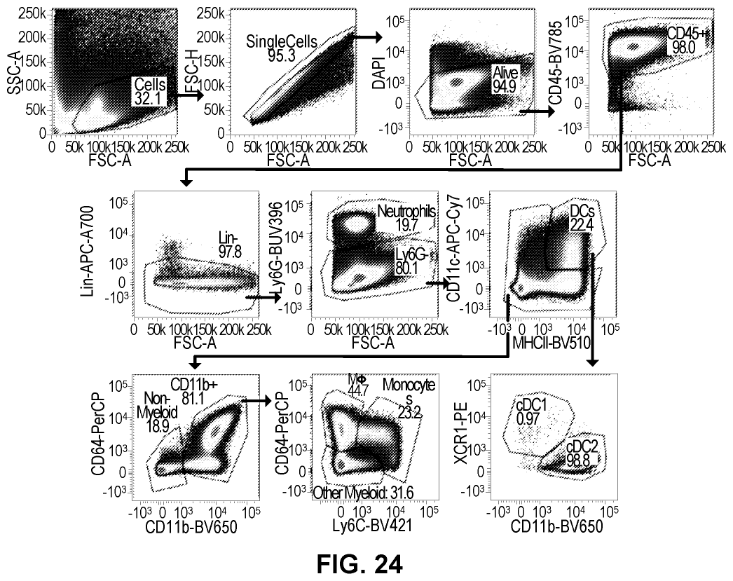

We evaluated the cells infiltrating the gel depots to understand if the gel was creating an inflammatory niche in addition to providing sustained release of the cargo (FIGS. 11A-11B). We quantified cell infiltration into the OVA and Poly(I:C) vaccine-loaded 2:10 gel compared to the 2:10 gel alone 7 days after SC injection in vivo with flow cytometry (FIG. 24). We found that the presence of vaccine cargo increased total cell infiltration into the gels, whereby almost 1.0×10⁶ total cells were found within a vaccine-loaded gel compared to 0.2×10⁶ cells in the control gel (FIG. 11C). The vaccine-loaded gel did not significantly recruit more neutrophils, but did recruit significantly higher numbers of monocytes, macrophages, and dendritic cells than an control gel (FIGS. 11D-11G)⁴⁴. Within the dendritic cell population found in the vaccine-loaded gel, the majority were migratory type 2 conventional DCs (cDC2), which play a critical role in activating follicular T helper cells (Tfh) and initiating the humoral immune response (FIG. 11H)⁴⁵. OVA uptake by cDC2s was confirmed via flow cytometry using Alexa Fluor 647 conjugated OVA as part of the vaccine (FIG. 11I). Diverse cell populations were observed in both the vaccine loaded gel and control gel, including neutrophils, monocytes, macrophages, dendritic cells, as well as other myeloid and non-myeloid cells (FIG. 11J). The initiation of the adaptive immune response relies on signaling from APCs, therefore the abundance of APCs (i.e., macrophages and DCs) in the vaccine-loaded gel at this early timepoint suggests that the gel acts as a beneficial inflammatory niche for immune activation.

### Germinal Center Response to Vaccination

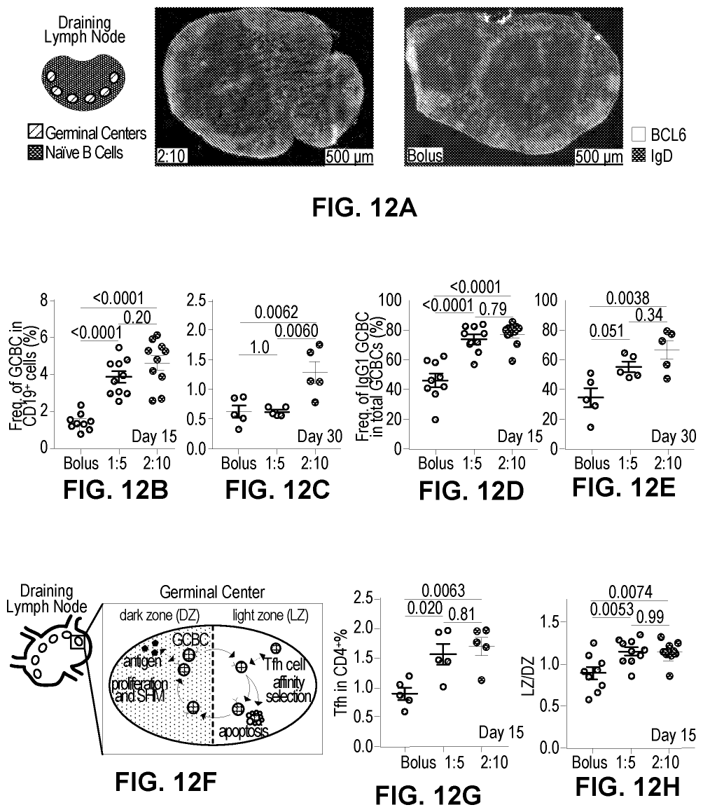

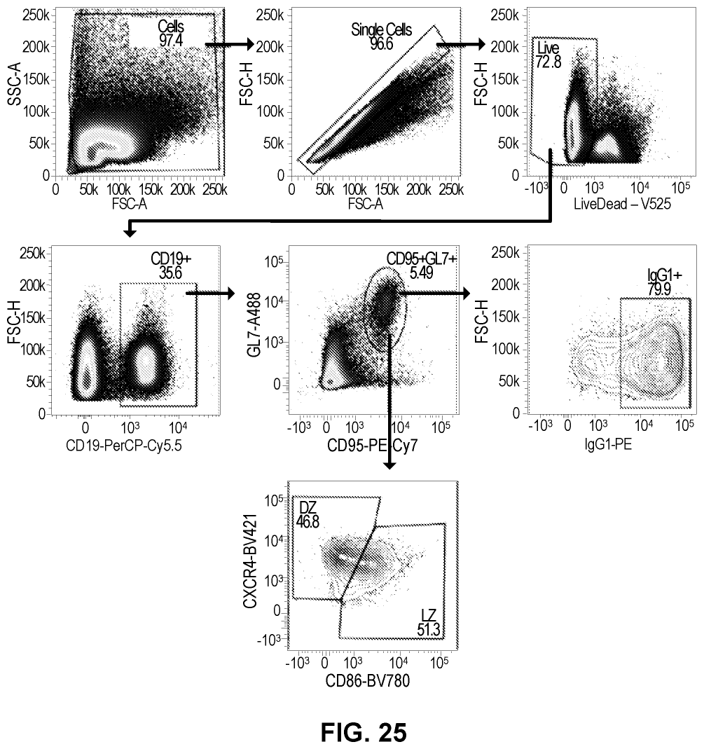

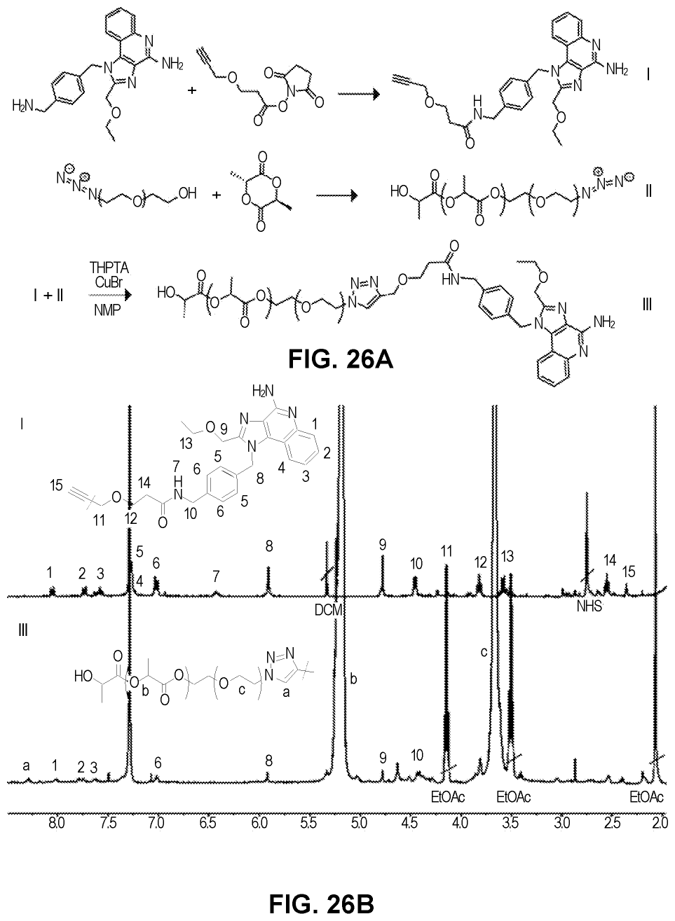

We hypothesized that the enhanced humoral response observed for gel-based sustained-exposure vaccines was due to the prolonged lifetime of GCs in the lymph nodes. GCs are dynamic sites that form after the activation of germinal center B cells (GCBCs) and are responsible for producing memory B cells and high-affinity antibodies³⁴. The GCs of the 2:10 and bolus groups 15 days after vaccination with OVA and Poly(I:C) were qualitatively visualized with immunohistochemistry (FIG. 12A). To quantitatively evaluate the GC response, we measured the frequency of GCBCs in the draining lymph nodes 15 and 30 days after vaccination with OVA and Poly(I:C) (FIG. 25). At 15 days after vaccine administration, mice in both the 1:5 and 2:10 gel groups had significantly higher frequencies of GCBCs than the mice who received the vaccine in PBS (FIG. 12B). At 30 days post-vaccination, mice that received the 2:10 gel formulation continued to have a higher GCBC frequency (FIG. 12C). Moreover, the 1:5 and 2:10 gel groups had a higher frequency of class-switched GCBCs (IgG1⁺) compared to the bolus group at day 15 (FIG. 12D); this is a critical indicator of a protective humoral respnonse^{34,46}. At day 30, the percent of class-switched GCBCs remained higher for the 2:10 group compared to the vaccine in PBS (FIG. 12E). Tfh cells play a critical role in GCBC selection, class-switching, and differentiation, and GCBCs interact with Tfh cells mainly in the light zone (LZ) of the GC (FIG. 12F)⁴⁷. In agreement with the GCBC results, we found an increase in the frequency of Tfh cells in the lymph nodes of both gel groups compared to bolus administration at the 15-day timepoint (FIG. 12G). The gel also led to an increase in the ratio of light zone to dark zone (LZ/DZ) GCBCs compared to bolus administration (FIG. 12H). These data indicate an increase in affinity selection compared to expansion and somatic hypermutation³⁴ Together, these data suggest that the prolonged vaccine exposure from the gels enhanced the magnitude and duration of the GC response and antibody affinity maturation.

### Sustained Influenza Vaccination

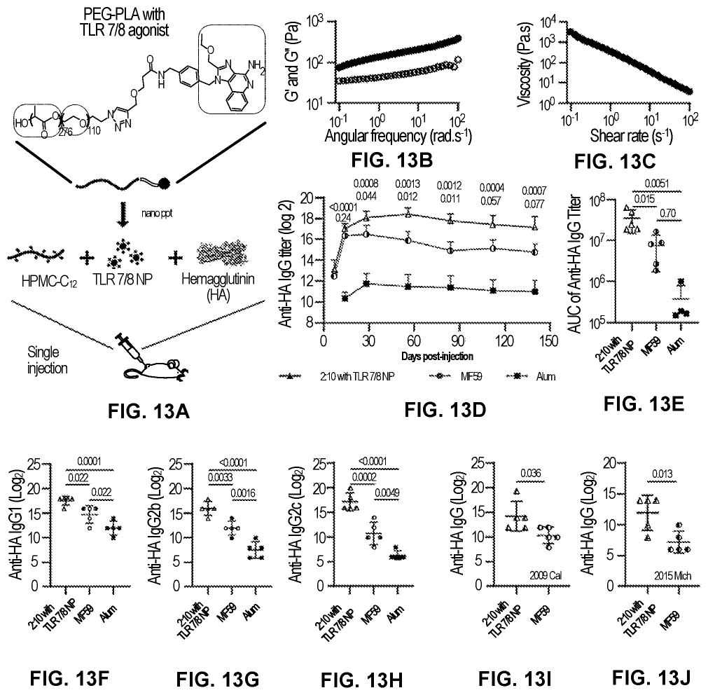

To demonstrate that our gel platform can be optimized for real viral antigens, we used the influenza antigen hemagglutinin (HA), which is the most commonly used antigen in influenza subunit vaccines. In these studies, we used a TLR 7/8 agonist adjuvant because it has been previously shown to elicit strong titers against HA and has demonstrated promise for clinical translation⁴⁸. To ensure sustained co-administration of HA and the TLR 7/8 agonist, which is a small molecule (314 Da), we tethered the TLR 7/8 agonist to the PEG-PLA NPs that form the PNP hydrogel network together with HPMC-C₁₂ (FIG. 13A, FIG. 16, Table 5). The TLR 7/8 NPs were formulated into 2:10 gels due to the improved efficacy seen with this formulation in the delivery of OVA-based vaccines. PNP hydrogels formulated with TLR 7/8 NPs exhibited similar rheological properties to gels prepared with standard PEG-PLA NPs (FIGS. 13B-13C).

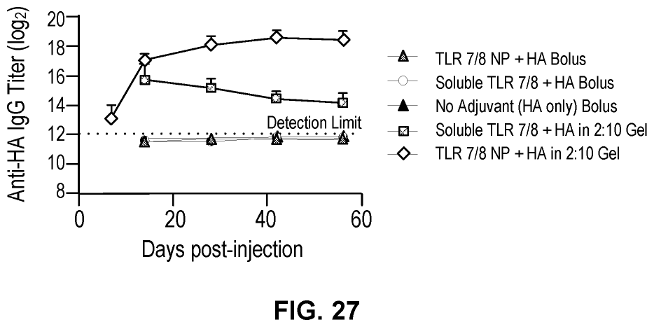

Next, to interrogate our material's ability to improve humoral immunity towards the clinically relevant HA antigen, we quantified the HA-specific IgG antibody titers after single administration of HA delivered in the TLR 7/8 NP-based 2:10 gel compared with clinically relevant adjuvants MF59, which is the most potent influenza vaccine adjuvant used clinically, and Alum. We showed that the TLR 7/8 NP-based gels led to 3.7-fold and 100-fold higher antibody titers compared to MF59 and Alum, respectively. Additionally, mice receiving the gel-based vaccine maintained antibody titers above the MF59 group's peak titer for over 140 days (FIG. 13D). The increased area under the curve (AUC) of the titers over time demonstrates that the gel vaccine formulation led to more potent and durable antigen-specific humoral immune responses compared to MF59 and Alum (FIG. 13E). We found that the TLR 7/8 NP-based gel performed better than the TLR 7/8 NPs alone as a bolus as well as soluble TLR 7/8 agonist (R848) in the gel (FIG. 27). Further characterization of the IgG subclasses 56 days after single administration shows that the gel delivery led to a significant increase in IgG1, IgG2b, and IgG2c antibodies compared to both adjuvant controls (FIGS. 13F-13H). Notably, we saw a 66-fold increase in IgG2c titers for the gel-based vaccine compared to MF59, which is the most important IgG subclass in C57BL/6 mice for fighting viral infections.

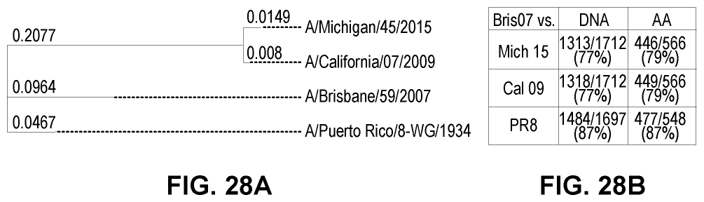

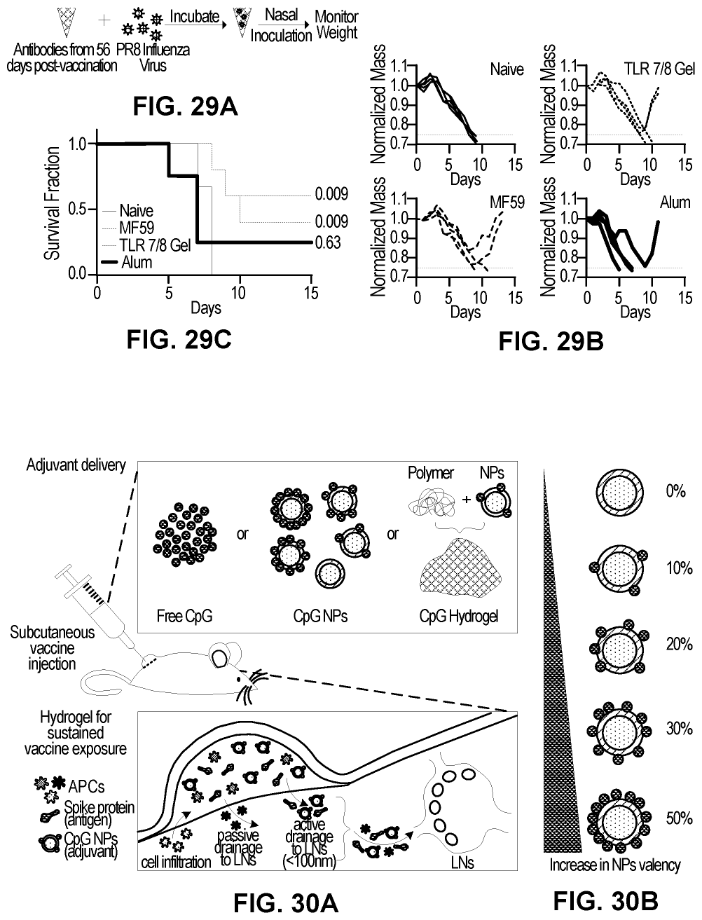

To evaluate cross reactivity to HA variants not included in the vaccine formulation, total IgG titers against HA from A/California/07/2009(H1N1) (Cal09) and A/Michigan/45/2015(H1N1) (Mich15) were quantified (FIGS. 13I-13J, FIG. 28). Delivery of A/Brisbane/59/2007(H1N1) HA in TLR 7/8 NP-based gel led to a 15-fold and 28-fold increase in titers against Cal09 and Mich15 HA variants compared to delivery with MF59 adjuvant, respectively, indicating that prolonged delivery of HA and a TLR 7/8 agonist in our hydrogel platform increases the breadth of the antibody response (FIGS. 13I-13J). Furthermore, the antibodies produced 56 days after vaccination were protective against a non-homologous viral challenge with the PR8 strain of influenza in an in vivo passive transfer model (FIG. 29). These studies demonstrate the ability of our PNP hydrogel platform to be used for augmenting and enhancing humoral immunity towards viral antigens.

### Cargo Diffusivity in PNP

The diagrams and diffusivity values in FIG. 17 show that in PBS the molecular size of a given cargo dictates its diffusivity according to the Stokes-Einstein equation, leading to approximately 10-fold different diffusivities for OVA and Poly(I:C) since they are approximately 10-fold different in size. When entrapped in a covalent PEG gel, the smaller cargo moves freely while the much larger Poly(I:C) is hindered by the mesh of the polymer network, leading to an even larger discrepancy in the diffusivities of the two cargo. In contrast, when encapsulated within the 1:5 and 2:10 PNP gels, both OVA and Poly(I:C) are hindered by the polymer network and their diffusion is therefore limited by the self-diffusivity of the dynamic PNP hydrogel network. Indeed, both cargo exhibit significantly slower rates of diffusion than are observed in PBS because of the obstruction the polymer network poses to their diffusion. When encapsulated in the 2:10 gel in particular, these two biomolecules exhibit matched diffusivities, despite their approximate 10-fold difference in size, as both cargo are completely hindered by the PNP hydrogel matrix. These findings suggest that when a hydrogel matrix is constraining to the diffusivity of entrapped cargo, the hydrogel self-diffusivity can be used as a powerful design principle for precisely engineering desired cargo diffusion properties within dynamically crosslinked supramolecular hydrogels.

### Germinal Center Response Overview

Germinal centers (GCs) are sites within lymphoid organs where mature B cells undergo somatic hypermutation (SHM) leading to higher affinity antibodies. Within the GCs, mature B cells go through cycles of proliferation, mutation, and selection in order to create B cells with the highest affinity B cell receptors (BCRs). The GC is divided into two anatomical compartments: the light zone (LZ) and the dark zone (DZ). In the LZ, GC B cells compete to capture antigen from follicular dendritic cells (FDCs) and present antigen peptides on MHCII2. The GC B cells with the highest affinity BCRs have increased antigen presentation to the T follicular helper (Tfh) cells and therefore will receive more positive signaling from Tfh cells. Tfh cell signaling is critical for positive selection of B cells with BCRs which have high affinity for antigen. The B cells that successfully interact with Tfh either mature into plasma cells or return the DZ for another round of proliferation and additional SHM^{57,58}. For this reason, prolonged Tfh cell presence in GCs is indicative of prolonged SHM. Similarly, a shift towards LZ B cell markers indicates an increase in the process of affinity selection compared to the processes of expansion and SHM which is necessary to continuing B cell cycling through the GC4.

### Rationale for Influenza Vaccine Model

The polymer-nanoparticle (PNP) gels described herein are formed by simple mixing of two aqueous solutions comprising (i) “polymer” (HPMC-C₁₂) and (ii) “nanoparticles” (PEG-PLA NPs). Upon mixing, a hydrogel rapidly forms as multivalent and dynamic non-covalent interactions between the HPMC-C₁₂ polymer and the PEG-PLA NPs generate physical cross-links that give rise to a polymer network structure. This hydrogel network structure provides the sustained vaccine exposure properties described herein. These PNP hydrogels act as a simple platform for encapsulation of a diverse array of vaccine components of interest and enhance humoral immune responses by altering the timeframe of vaccine presentation to the immune system. Our study focuses on the material platform's ability to manipulate the humoral immune response, regardless of the particular vaccine, by first using a model vaccine consisting of ovalbumin (OVA) and Poly(I:C) and then using an influenza vaccine consisting of hemagglutinin (HA) and an R848 derivative. The studies with HA immunization demonstrate the ability of the PNP hydrogel to be used for slow delivery of a “real” viral antigen and reveals the broad applicability of our delivery platform as the molecular weights and physicochemical properties of OVA, Poly(I:C), R848, and HA are very diverse. It is important to note that an optimized and highly effective vaccine requires three parts: (i) advanced antigen design, (ii) potent adjuvants, and (iii) appropriate delivery characteristics.

Several adjuvant technologies have been used in commercial influenza vaccines, including MF59, Al(OH)3, and AS03. The only adjuvanted seasonal flu vaccine is FLUAD® (Seqirus Inc), which contains MF59 and is used for persons 65 years of age and older⁶⁰. The remaining licensed adjuvanted flu vaccines all target the H5N1 pandemic influenza strain. These formulations include MF59 in AUDENZ (Seqirus), Al(OH)3 in Panflu (Sinovac), and AS03 in Prepandrix (GSK) and Q-Pan H5N1 (GSK)⁶⁰. Yet, studies indicate that adjuvants alone may not provide sufficient immunological driving forces for promoting extensive affinity maturation, suggesting the kinetics of vaccine exposure must be carefully considered. Recently, Crotty and coworkers demonstrated that slow release of an HIV vaccine using surgically implantable pump leads to more robust germinal center responses, higher antibody titers, and the targeting of a more diverse set of epitopes than standard bolus administration of the same vaccine⁶¹. Sustained exposure can therefore enhance neutralizing antibody development by altering germinal responses and modulating the immunodominance of non-neutralizing epitopes.

For these reasons, we do not argue that we have designed an optimized influenza vaccine as we use a simple, commercially available HA antigen and compare our sustained delivery hydrogel system to current clinically used adjuvants. Our study indicates the ability to improve the humoral immune response to the HA viral antigen by choosing an appropriate adjuvant and using the PNP hydrogel platform to provide prolonged co-delivery of antigen and adjuvant.

### Discussion

In this study, we designed an injectable hydrogel material capable of prolonged co-delivery of physicochemically-distinct cargo on timescales relevant to the kinetics of antigen exposure typical of natural infections. The vaccine-loaded PNP hydrogels are injectable and retain their solid-like structure when under low stresses, enabling creation of a new stimulatory microenvironment within the body while facilitating sustained vaccine delivery. Additionally, the dynamic networks comprising the 1:5 and 2:10 gels provide unique cargo diffusion characteristics compared to traditional covalent hydrogel systems. Our observations suggest that the 2:10 gel mesh sufficiently constrains the diffusion of both OVA and Poly(I:C), even though Poly(I:C) is much larger than OVA, such that their diffusion and release is dictated by the self-diffusion of the dynamically cross-linked hydrogel network. These physical properties ensure co-presentation of antigen and adjuvant over prolonged timeframes to the immune system which has been a long-standing challenge within the field.

Developing effective humoral immune responses towards difficult pathogens, such as HIV-1 or malaria, requires the creation of high affinity antibodies^{49,50}. The sustained vaccine exposure enabled by these gels was shown to increase the magnitude and duration of GC responses in the draining lymph nodes, leading to enhancements in the magnitude, persistence, and quality of the humoral immune response against the antigen. The sustained release of antigen from PNP hydrogels is able to better mimic natural infections where GCBCs continuously receive antigen-derived signaling to undergo many rounds of selection and somatic hypermutation (FIG. 8C). In addition, sustained vaccine release provides GCBCs the essential B cell receptor signaling needed to promote the DZ to LZ transition and positive selection of high affinity GCBCs². The significantly higher affinity antibodies produced in mice immunized with gel-based vaccines suggest an increase in somatic hypermutation and commensurate enhancement in affinity maturation.

These gels also act as a local stimulatory microenvironment where infiltrating cells experience high local concentrations of adjuvant and antigen. Implantable biomaterials have been shown to act as immune-stimulating niches which recruit and activate APCs that then migrate to the lymph nodes⁵¹. Herein, we observed minimal immune cell infiltration in the gels alone; however, loading the gels with vaccine cargo dramatically increased immune cell infiltration, which is likely driven by the inflammatory signals initiated by the entrapped adjuvant. The vaccine-loaded gel was able to recruit macrophages and DCs, which are professional APCs that are essential for initiating the adaptive immune response. In particular, almost all infiltrating DCs were found to be cDC2s which are known to play a critical role in activating Tfh cells and initiating the humoral immune response. We hypothesize that these cells are activated by entrapped adjuvant and migrate to the draining lymph nodes. The draining lymph nodes are therefore receiving activated immune cells from the gel as well as the soluble vaccine cargo as it is released from the gel depot over time (FIGS. 8B-8C).

### Conclusion

In conclusion, PNP hydrogels provide a simple and effective platform for sustained delivery of subunit vaccines to increase the potency and durability of the humoral immune response. Using our platform as a tool to probe the interactions between the immune system and a vaccine depot will enable more precise material development for vaccine delivery. Our PNP hydrogel represents a highly tunable platform for effectively manipulating the humoral immune response for any subunit vaccine of interest.

### Methods

Materials HPMC (meets USP testing specifications), N,N-Diisopropylethylamine (Hunig's base), hexanes, diethyl ether, N-methyl-2-pyrrolidone (NMP), dichloromethane (DCM), lactide (LA), 1-dodecylisocynate, and diazobicylcoundecene (DBU) were purchased from Sigma Aldrich and used as received. Monomethoxy-PEG (5 kDa) was purchased from Sigma Aldrich and was purified by azeotropic distillation with toluene prior to use.

Preparation of HPMC-C₂: HPMC-C₁₂ was prepared according to previously reported procedures²⁸. HPMC (1.0 g) was dissolved in NMP (40 mL) by stirring at 80° C. for 1 h. Once the solution cooled to room temperature, 1-dodecylisocynate (105 mg, 0.5 mmol) and N,N-Diisopropylethylamine (catalyst, ˜3 drops) were dissolved in NMP (5.0 mL). This solution was added dropwise to the reaction mixture, which was then stirred at room temperature for 16 h. For rhodamine conjugated HPMC-C₁₂, 10 mg of rhodamine B isothiocyanate (0.019 mmol) was added to the 1-dodecylisocyanate solution before adding it dropwise to the reaction mixture. This solution was then precipitated from acetone, decanted, re-dissolved in water (˜2 wt %), and placed in a dialysis tube for dialysis for 3-4 days. The polymer was lyophilized and reconstituted to a 60 mg/mL solution with sterile PBS.

Preparation of PEG-PLA NPs and TLR 7/8 NPs: PEG-PLA was prepared as previously reported²⁸. Monomethoxy-PEG (5 kDa; 0.25 g, 4.1 mmol) and DBU (15 μL, 0.1 mmol; 1.4 mol % relative to LA) were dissolved in anhydrous dichloromethane (1.0 mL). LA (1.0 g, 6.9 mmol) was dissolved in anhydrous DCM (3.0 mL) with mild heating. The LA solution was added rapidly to the PEG/DBU solution and was allowed to stir for 10 min. The reaction mixture was quenched and precipitated by 1:1 hexane and ethyl ether solution. The synthesized PEG-PLA was collected and dried under vacuum. Gel permeation chromatography (GPC) was used to verify that the molecular weight and dispersity of polymers meet our quality control (QC) parameters.

Azide-PEG-PLA was prepared using N₃-PEG-OH (0.5 g, 5 kDa, 100 μmol) in 2 mL of anhydrous DCM with 30 μL DBU (30 mg, 0.20 mmol) which was added quickly to a stirring solution of LA (2 g, 13.9 mmol) in 6 mL anhydrous DCM. The solution was stirred for 10 min, after which 2 drops of acetic acid was added, and the polymer was precipitated into a 1:1 mixture of hexanes and diethyl ether. The polymer was re-dissolved in a minimal amount of acetone, and precipitated again in diethyl ether, and dried in vacuo. GPC was used to verify that the molecular weight and dispersity of polymers meet our QC parameters.

A 20 mL scintillation vial was charged with the TLR 7/8 agonist alkyne (14 mg, 30 μmol) and azido poly(ethylene oxide)-b-poly(D,L-lactide) 5 kDa-20 kDa (0.5 g, 20 umol) was dissolved in 4 mL of NMP and sparged with nitrogen for 10 min. Next, 0.1 mL of a degassed CuBr (3.7 mg/mL) and THPTA (16 mg/mL) was added. The reaction mixture was further sparged with nitrogen gas for 10 min. The reaction mixture was incubated for 16 h at room temperature, and precipitated into diethyl ether in a 50 mL centrifuge tube to recover the polymer. The polymer was then dissolved in ethyl acetate and precipitated into diethyl ether, and dried in vacuo. GPC was used to verify that the molecular weight was not altered by conjugation.

NPs were prepared as previously reported²⁸. A 1 mL solution of PEG-PLA in DMSO (50 mg/ml) or TLR 7/8-PEG-PLA in acetonitrile (50 mg/mL) was added dropwise to 10 mL of water at room temperature under a high stir rate (600 rpm). NPs were purified by ultracentrifugation over a filter (molecular weight cut-off of 10 kDa; Millipore Amicon Ultra-15) followed by resuspension in water to a final concentration of 200 mg/mL. NPs were characterized by dynamic light scattering (DLS) to find the NP diameters, 32±4 nm and 29±3 nm, and zeta potential, −28±7 mV and −10±7 mV, for the PEG-PLA and TLR 7/8-PEG-PLA NPs, respectively (Table 6).

PNP Hydrogel Preparation. The 2:10 formulation contained 2 wt % HPMC-C₁₂ and 10 wt % PEG-PLA NPs in PBS. These gels were made by mixing a 2:3:1 weight ratio of 6 wt % HPMC-C₁₂ polymer solution, 20 wt % NP solution, and PBS. The 1:5 formulation contained 1 wt % HPMC-C₁₂ and 5 wt % PEG-PLA NPs in PBS. These gels were made by mixing a 2:3:7 weight ratio of 6 wt % HPMC-C₁₂ polymer solution, 20 wt % NP solution, and PBS. The solutions were mixed with a spatula, mildly centrifuged to remove bubbles arising from mixing, and then loaded into a syringe.

Vaccine Formulations: The model vaccine contained a 100 μg dose of OVA (Sigma Aldrich) and 50 μg dose of Poly(I:C) (Sigma Aldrich) per 100 μL of gel or PBS. The influenza vaccine contained a 2 μg dose of Influenza A HIN1 (A/Brisbane/59/2007) hemagglutinin (HA)(Sino Biological) and an approximate TLR 7/8 agonist dose of 50 μg. For the bolus vaccines, the above vaccine concentrations were prepared in PBS and loaded into a syringe for administration. For the PNP hydrogels, the vaccine cargo was added at the appropriate concentration into the PBS component of the gel before adding the polymer and NP solutions, as described above. For MF59 (Invivogen) and Alum (Invivogen) vaccines, the formulations were prepared according to the manufacturer's instructions with a 2 μg dose of HA.

Material Characterization: Rheological characterization was performed using a TA Instruments Discovery HR-2 torque-controlled rheometer fitted with a Peltier stage. All measurements were performed using a serrated 20-mm plate geometry at 25° C.

Dynamic oscillatory frequency sweep measurements were performed with a constant torque (2 uN·m; σ=1.27 Pa) from 0.1 rad/s to 100 rad/s. Steady shear experiments were performed from 0.1 to 100 s⁻¹. Step-shear experiments were performed by alternating between a low shear rate (0.05 s⁻¹) and high shear rate (100 s⁻¹) for two full cycles.

FRAP Analysis: Alexa Fluor 647 conjugated OVA (Thermo Fisher Scientific), rhodamine conjugated Poly(I:C) (Invivogen), and rhodamine conjugated HPMC-C₁₂ were used to visualize the diffusion of the cargo and gel. Samples were photobleached with a 50 μm diameter for the region of interest (ROI). Different tests (n=5) were made for 3 different samples from the same batch at different locations of the sample. A spot was bleached with a pixel dwell time of 177.32 μs. 500 post-bleach frames were recorded at 1 frame/s to form the recovery exponential curve. The diffusion coefficient was calculated as⁵²:

D=γD(ω²/4Tτ_{1/2})

Where the constant γD=τ_{1/2}τD, with τ_{1/2} being the half-time of the recovery. τ_{D} the characteristic diffusion time, both yielded by the ZEN software, and ω the radius of the bleached ROI (25 μm).

The diffusivity of cargo in PBS was calculated using the Stokes-Einstein Law Equation for diffusion⁵³ where k_{B} is Boltzmann's constant, T is temperature in Kelvin, η is solvent viscosity, and R is solute hydrodynamic radius

$$D = \frac{{k}_{B} T}{6 πη R}$$

The diffusivity of cargo in a model covalent PEG gel was calculated using the Multiscale Diffusion Model (MSDM) assuming 25° C., 5% volume fraction, and 35 nm mesh size⁵⁴. The calculated values are comparable to experiment diffusivities of similar sized cargo found in the literature⁵⁵.

Mice and Vaccination: C57BL/6 (B6) mice were purchased from Charles River and housed at Stanford University. Female mice between 6 and 10 weeks of age at the start of the experiment were used. The mice were shaved several days before vaccine administration and received a subcutaneous injection of 100 μL gel or bolus vaccine on their backs under brief isoflurane anesthesia. PBS injections used a 26-gauge needle, and gel injections used a 21-gauge needle. Mouse blood was collected from the cheek or tail vein for survival studies, or through cardiac puncture for terminal studies. The inguinal LN's and SC gels were collected for GC and cell infiltration analysis after euthanasia.

In Vivo Cargo Dynamics: Vaccine loaded gels with Alexa Fluor 647 conjugated OVA (Thermo Fisher Scientific), were explanted at 1, 7, 14, 21, or 28 days, and weighed to report the gel erosion over time. After weighing, the explanted gels were diluted in PBS and homogenized using a glass dounce homogenizer (Wheaton). The fluorescence of the homogenized gels was read with ex: 650 nm and em: 665 nm on a plate reader (Tecan Infinite M1000). Raw fluorescence values were normalized for polymer background, the natural log of these values was plotted and fit with linear equations to find the rate constant using GraphPad Prism 7.04 (GraphPad Software). This rate constant was used to calculate a half-life of cargo retention.

Antibody Concentration and Affinity: Serum antibody concentrations and affinity for the OVA model vaccine were measured using an anti-ovalbumin mouse IgG1 ELISA (Cayman Chemicals. 500830). For time course measurements the serum was diluted 1:1,000 in assay buffer and for post-challenge analysis the serum was diluted 1:100,000. The assay was performed according to the manufacturer's instructions to find concentration. The plates were analyzed using a Synergy™ H1 Microplate Reader (BioTek Instruments) at 450 nm. Serum antibody concentrations were calculated from the standard curves and represented as μg/mL or mg/mL.

Anti-OVA IgG2b and IgG2c antibody concentrations were measured using mouse anti-OVA antibody assay kits for IgG2b (chondrex, 3016) and IgG2c (Chondrex, 3029). Serum was diluted 1:1,000 and the assay was performed according to the manufacturer's instructions to find concentration. The plates were analyzed using a Synergy™ H1 Microplate Reader (BioTek Instruments) at 450 nm. Serum antibody concentrations were calculated from the standard curves and represented as μg/mL.

For affinity, dilutions of the serum were mixed with a constant 3 nM of an HRP conjugated anti-OVA antibody (BioLegend) and incubated for 2 h at room temperature. The wells were washed, incubated with TMB substrate (TMB ELISA Substrate (High Sensitivity). Abcam), and the reaction was stopped with 1 M HCl. Absorbance was read with a plate reader at 450 nm. Data was fit with a one-site competitive binding model with Graphpad Prism. The control antibody was assumed to have a K_{D} of 1 nM based on common affinities of mAbs found in the industry. This assumption affects only the absolute K_{D} values reported and not the relative differences between treatment groups. Statistics were performed on the log₁₀ (K_{D}) values. Individual binding curves can be found in FIG. 16.

Serum IgG antibody titers for the influenza vaccine were measure using an ELISA. Ni-coated plates (Thermofisher) were coated with HA (Sino Biological) at 2.5 μg/mL in PBS for 1 h at 25° C. and blocked with PBS containing 1% BSA for 1 h at 25° C. A standard curve was created by pooling serum and completing serial dilutions (2×) before adding to the plate and serum samples were diluted 1:200 (Alum group) or 1:1,000 (Gel and MF59 groups) and added to plates. After 2 h at 25° C., goat-anti-mouse IgG Fc-HRP (1:10,000, Invitrogen, A16084) was added for 1 h at 25° C. Plates were developed with TMB substrate (TMB ELISA Substrate (High Sensitivity), Abcam). The reaction was stopped with 1 M HCl. The plates were analyzed using a Synergy™ H1 Microplate Reader (BioTek Instruments) at 450 nm. Serum antibody titers were calculated from the standard curve and represented as the dilution required to reach the detection limit.

Serum IgG1, IgG2b, and IgG2c antibody titers against A/Brisbane/59/2007 HA and IgG titers against A/California/07/2009 and A/Michigan/45/2015 HA were measure using an endpoint ELISA. Ni-coated plates (Thermofisher) were coated with HA (Sino Biological) at 2.5 μg/mL in PBS for 1 h at 25° C. and blocked with PBS containing 1% BSA for 1 h at 25° C. First, serum was diluted 1:250 and then four-fold serial dilutions were performed up to 1:4,096,000 dilution. Titrations were added to plates and after 2 h at 25° C., HRP-conjugated goat anti mouse IgG1 (Abcam, ab97240), IgG2b (Chondrex, 3016), IgG2C (Abcam, ab97255), or IgG (Invitrogen, A16084) were added at a 1:10,000 dilution for 1 h at 25° C. Plates were developed with TMB substrate (TMB ELISA Substrate (High Sensitivity), Abcam). The reaction was stopped with 1 M HCl. The plates were analyzed using a Synergy™ H1 Microplate Reader (BioTek Instruments) at 450 nm. Endpoint titers were defined as the reciprocal of the highest serum dilution that gave an optical density above 0.1.

Immunohistochemistry in LN: Draining inguinal lymph nodes were isolated and frozen in molds containing OCT medium on dry ice. Frozen lymph nodes were sectioned at 7 μm and stored at −80° C. Sections were stained with biotin labeled anti mouse IgD (eBioscience) and PE labeled anti mouse BCL6 (BD Biosciences) antibodies, and subsequently stained with A1488 conjugated Streptavidin (Thermo Fisher Scientific). Fluorescent images were captured using a 20×objectives on a fluorescence microscope (Keyence). There were fundamental limitations in the quantity and orientation of GCs in the LNs, so immunohistochemistry was not used to quantify cell types or GC features.

Immunophenotyping in LN: Inguinal lymph nodes were surgically removed from the animals after euthanasia and then were mechanically disrupted to create a cell suspension. For FACS analysis, cells were blocked with Fc receptor antibody (clone: 2.4G2, Tonbo Biosciences) and then stained with fluorochrome conjugated antibodies: CD19, GL7, CD95, CXCR4, CD86, IgG1, CD4, CXCR5, and PD1. After staining, cells were washed and fixed with 1.5% paraformaldehyde (PFA). Stained cells were analyzed on LSRII flow cytometer. Data were analyzed with FlowJo 10 (FlowJo LLC). See. FIG. 24 for gating strategy and Table 7 for the antibody panel.

Immunophenotyping in Gel: Gels were surgically removed from the animals after euthanasia. The gels were processed using mechanical disruption to create a cell suspension which was then filtered through a 70 μm cell strainer (Celltreat). To count total cells in the gels, the single-cell suspensions were stained with anti-mouse CD45 and DAPI to count live leukocytes. The cells were mixed with CountBright Absolute Counting beads (ThermoFisher) and analyzed on a LSRFortessa X-20 flow cytometer (BD Biosciences). To analyze neutrophil and other myeloid cell counts in the gels, the single-cell suspensions were incubated with anti-CD16/CD32 (produced in house from 2.4G2 hybridoma) to block Fc receptor binding, and then stained with anti-mouse CD45, CD3, CD19, CD11c, Ly6G, CD11b, and MHCII in FACS buffer (2 mM EDTA, 2% FBS in PBS) for 30 min on ice. Dead cells were excluded by DAPI staining. Cells were acquired on a LSRFortessa X-20 and fcs files were analyzed using FlowJo 10 (FlowJo LLC). See FIG. 23 for gating strategy and Table 7 for the antibody panel.

Animal Protocol: All animal studies were performed in accordance with National Institutes of Health guidelines, with the approval of Stanford Administrative Panel on Laboratory Animal Care.

Statistical Analysis: All results are expressed as a mean±standard deviation (s.d.) except for FIG. 10B which is mean±standard error of the mean (s.e.m.) as indicated in the figure captions. Comparisons between two groups were conducted by a two-tailed Student's t-test. One-way ANOVA test with a Tukey's multiple comparisons test was used for comparison across multiple groups. Statistical analysis was run using GraphPad Prism 7.04 (GraphPad Software). Statistical significance was considered as p<0.05.

FRAP Analysis: Alexa Fluor 647 conjugated OVA (Thermo Fisher Scientific), rhodamine conjugated Poly(I:C) (Invivogen), and rhodamine conjugated HPMC-C₁₂ were used to visualize the diffusion of the cargo and gel. The samples were placed in a sterile 0.18 mm thick glass bottom dish (Ibidi). An Inverted Zeiss LSM 780 Laser Scanning Confocal Microscope (Germany) using a Plan-Apochromat 20×/0.8 M27 objective lens was used for FRAP measurements. To excite the fluorescent Alexa Fluor 647, a 5 mW 633 nm He—Ne laser was employed at 2%, and the emitted fluorescence was detected by Alexa Fluor 647 specific band pass filter (638-756 nm). To excite the fluorescent rhodamine, a 20 mW 561 nm diode pumped solid state laser was used at 2%, and the emitted fluorescence was detected by rhodamine specific band pass filter (415-638 nm). We used pixel dwell time 2.55 μs which took 390.98 ms to finish every scan. Samples were photobleached with a 405 nm diode laser, plus the 633 nm laser in the case of the Alexa Fluor, and a 514 nm argon laser and the 561 nm laser in the case of the rhodamine. All lasers were set al 100% intensity for the bleaching, with a 20-40 μm diameter for the region of interest (ROI). To avoid any extra noise, the high voltage was limited to be 700 V. Different tests (n=5) were made for 3 different samples from the same batch at different locations of the sample. For each test, 10 control pre-bleach images were captured at 1 frame/s. A spot was bleached with a pixel dwell time of 177.32 μs. 500 post-bleach frames were recorded at 1 frame/s to form the recovery exponential curve. The pixel size was set to be 1.66 μm. The diffusion coefficient was calculated as⁶²:

D=γD(ω²/4Tτ_{1/2})

Where the constant γ_{D}=τ_{1/2}/τ_{D}, with τ_{1/2} being the half-time of the recovery, TD the characteristic diffusion time, both yielded by the ZEN software, and ω the radius of the bleached ROI (25 μm). The software used for all FRAP tests was the ZEN lite (Zeiss). All the experiments were conducted in the Stanford University Cell Sciences Imaging Facility (CSIF) at room temperature.

R_{g} of Poly(I:C) was obtained from gel permeation chromatography (GPC) carried out using a Dionex Ultimate 3000 instrument (including pump, autosampler, and column compartment). Detection consisted of an Optilab TrEX (Wyatt Technology Corporation) refractive index detector operating at 658 nm and a HELEOS II light scattering detector (Wyatt Technology Corporation) operating at 659 nm. The column used was a Superose 6 increase 10/300 GL. The eluent was PBS buffer. 137 mM NaCl. 0.0027 mM KCl, 10 mM Phosphate pH 7.3, at 0.75 mL min⁻¹ at room temperature. Analyte samples at 2 mg mL⁻¹ were filtered through a PVDF membrane with 0.2 mm pore size prior to injection. R_{g} was calculated using a dndc of 0.19 mL/g and converted to R_{H} using the following equation⁶³: R_{g}/R_{H}≈0.77.

The diffusivity of cargo in PBS was calculated using Stokes-Einstein Law Equation for diffusion⁶⁴ where kₐ is Boltzmann's constant, T is temperature in Kelvin, η is solvent viscosity, and R is solute hydrodynamic radius:

$$D = \frac{{k}_{B} T}{6 πη R}$$

The diffusivity of cargo in a model covalent PEG gel was calculated using the Multiscale Diffusion Model (MSDM) assuming 25° C., 5% volume fraction, and 35 nm mesh size⁵⁶. The calculated values are comparable to experiment diffusivities of similar sized cargo found in the literature⁶⁵.

TLR small molecule synthesis: The benzylamine TLR 7/8 agonist was synthesized as described by Shukla et. al.⁶⁶, with a modification of the final aromatic substitution and tert-butyl carbamate removal on tert-butyl (4-((4-chloro-2-(ethoxymethyl)-1H-imidazo[4,5-c]quinolin-1-yl)methyl)benzyl)carbamate (1), which was done as a one-pot Staudinger-type reaction.⁶⁷

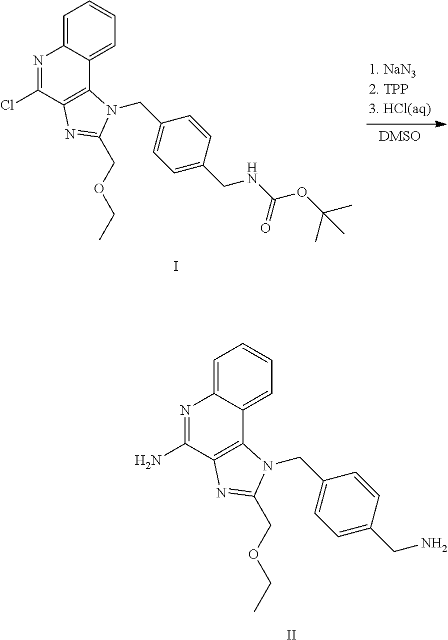

Compound I and 2 equivalents of NaN₃ (68 mg, 1.04 mmol) was dissolved in 1.5 mL DMSO, and was heated to 90° C. for 2 h. 2 equivalents of triphenylphosphine (273 mg, 1.04 mmol) was added, and the temperature was increased to 120° C. and stirred for 16 h. 4M HCl (aq; 0.5 mL) was added to the reaction flask and the temperature was lowered to 95° C. for 3 h. Water (4 mL) was added at which a precipitate formed, and the mixture was washed with EtOAc. NaCO₃ (sat) was added to the aqueous phase which was extracted with EtOAc three times, which was done with Na₂SO₄ and the solvent removed in vacuo to yield a crude solid. This was purified by silica column chromatography on a Biotage system, using a gradient of DCM:MeOH, with 1% trimethylamine in the MeOH. This yielded compound II (1-(4-(aminomethyl)benzyl)-2-(ethoxymethyl)-1H-imidazo[4,5-c]quinolin-4-amine) (60 mg, 0.16 mmol, 32% yield).

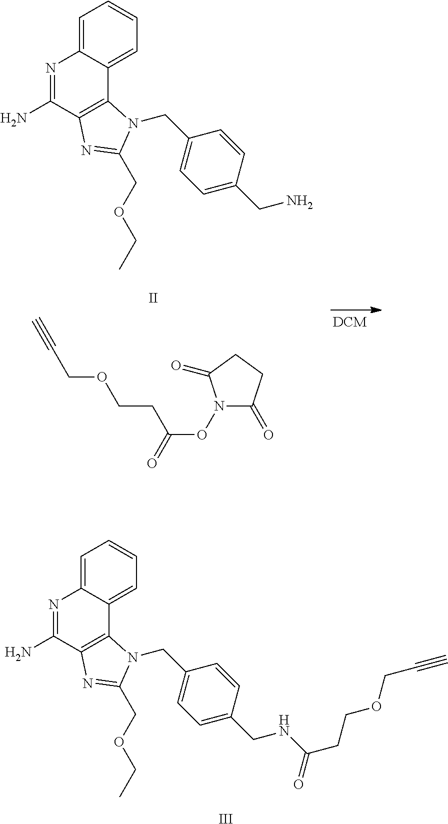

Compound II (25 mg, 69 μmol) and propargyl-N-hydroxysuccinimidyl ester (17 mg, 76 μmol) were dissolved in 1 mL anhydrous DCM, and stirred for 2 hours at room temperature. The reaction mixture was loaded onto a silica column, and the product was purified using a DCM:MeOH gradient. This yielded compound III N-(4-((4-amino-2-(ethoxymethyl)-1H-imidazo[4,5-c]quinolin-1-yl)methyl)benzyl)-3-(prop-2-yn-1-yloxy)propanamide (25 mg, 53 μmol, 76% yield).

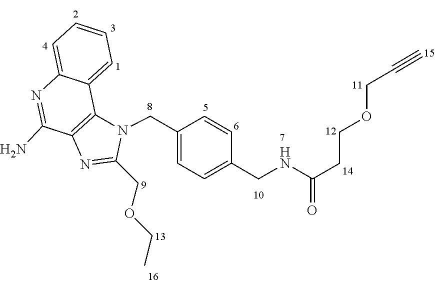 [SMILES: C#CCOCCC(=O)NCc1ccc(Cn2c(COCC)nc3c(N)nc4ccccc4c32)cc1]

¹H NMR (300 MHz, CDCl₃-d): δ 8.05 (d, J=8.4 Hz, 1H, H1), 7.73 (d, J=8.3 Hz, 1H, H2), 7.58 (t, J=7.9 Hz, 1H, H3), 7.29 (3H, H4, H5), 7.02 (d, J=7.9 Hz, 2H, H6), 6.43 (s, 1H, H7), 5.91 (s, 2H, H8), 4.78 (s, 2, H9), 4.46 (d, J=5.9 Hz, 2H, H10), 4.15 (d, J=2.4 Hz, 2H, H 11), 3.82 (t, J=5.7 Hz, 2H, H12), 3.58 (q, J=7.2 Hz, 2H, H13), 2.55 (t, J=5.7 Hz, 2H, H14), 2.36 (t, J=2.4 Hz, 1H, H15), 1.14 (t, J=7.2 Hz, 3H, H16).

¹³C NMR (75 MHz, CDCl₃): δ 171.35, 166.55, 151.77, 150.14, 138.70, 136.66, 136.09, 133.40, 129.84, 128.43, 125.71, 124.66, 121.24, 120.73, 112.79, 79.06, 75.01, 66.77, 66.02, 64.19, 58.44, 58.38, 58.21, 36.88, 31.89, 14.86.

Polymer synthesis and functionalization: N₃-PEG-OH (0.5 g, 5 kDa, 100 μmol) in 2 mL anhydrous DCM with 30 μL DBU (30 mg, 0.20 mmol) was added quickly to a stirring solution of lactide (2 g, 13.9 mmol) in 6 mL anhydrous DCM. The solution was stirred for 10 min, after which 2 drops of acetic acid was added to quench the polymerization, and the polymer was precipitated into a 1:1 mixture of hexanes and diethyl ether. The polymer was re-dissolved in a minimal amount of acetone, precipitated again in diethyl ether and dried in vacuo.

A 20 mL scintillation vial was charged with compound III (14 mg, 30 μmol) and azido-poly(ethylene oxide)-b-poly(D,L-lactide) 5 kDa-20 kDa (0.5 g, 20 μmol) was dissolved in 4 mL of NMP and sparged with nitrogen for 10 min. Degassed CuBr (3.7 mg/mL: 0.1 mL) and THPTA (16 mg/mL) was added to the reaction flask. The reaction mixture was further sparged with nitrogen for 10 min. The reaction mixture was incubated for 16 h at room temperature, and precipitated into diethyl ether in a 50 mL centrifuge tube to recover the polymer. The polymer was then dissolved in ethyl acetate, precipitated into diethyl ether and dried in vacuo.

Conjugation was confirmed by ¹H-NMR spectroscopy and increased UV absorption as indicated by GPC (DMF) eluagram.

NMR Spectroscopy: NMR spectra were obtained using an Inova 300 MHz NMR spectrometer with a Varian Inova console using VNMRJ 4.2 A software. Number-average (Mn) and weight-average (Mw) molar mass and dispersity (D=Mw/Mn) of polymers were obtained from gel permeation chromatography (GPC) carried out using an Dionex Ultimate 3000 instrument (including pump, autosampler, and column compartment) outfitted with an ERC Refractomax 520 refractometer. The columns were Jordi Resolve DVB 1000 Å, 5 m, 30 cm×7.8 mm and a Mixed Bed Low, 5 m, 30 cm×7.8 mm, with a Jordi Resolve DVB Guard Column, 1000 Å, 5 m, 30 cm×7.8 mm, 5 cm×7.8 mm. DMF with 10 mM LiBr was used as eluent at 1 mL min⁻¹ at room temperature. Poly (ethylene glycol) were used to calibrate the GPC system. Analyte samples at 2 mg mL⁻¹ were filtered through a nylon membrane with 0.2 mm pore size before injection (20 μL). Data was analyzed using Chromeleon GPC/SEC Software.

Surface Plasmon Resonance: A BIAcore X100 instrument (GE Healthcare) was used for all Surface Plasmon Resonance (SPR) kinetics measurements. Anti-mouse IgG antibodies (Cat. BR100838, GE Healthcare) were immobilized to a CM5 chip (Cat. BR100012, GE Healthcare) using ammine coupling according to the manufacturer's instructions. Serum samples were injected at a 1:100 dilution in HBS-EP+ buffer (Cat. BR 100826, GE Healthcare) for 140 seconds at a flow rate of 10 μL/min to capture the mouse antibodies onto the chip. For association analysis, 5 different concentrations of OVA in HBS-EP+ buffer (978 nM, 733 nM, 489 nM, 244 nM, 162 nM) were flowed for 90 seconds at 30 μL/min followed by 9×) seconds of buffer alone. For dissociation analysis, OVA at 489 nM was run for 90 seconds at 30 μL/min followed by 30 minutes of HBS-EP+ to allow for OVA to dissociate from the antibodies.

For each run, we had two steps of reference subtraction. First, naïve mouse serum was run in parallel to the test serum in the reference cell of the chip and subtracted from all test curves. Second, buffer was run above the captured antibodies with the same settings as the OVA solutions to subtract for temporal changes due to fluid flow.

To calculate the association rate constant, GraphPad Prism 7.04 (GraphPad Software) was used to fit a one-phase association model to all 5 concentrations of OVA. The observed k values from those fits were plotted against the concentration and fit with a linear equation. The slope from this line was defined as the association rate for that sample.

To calculate the dissociation rate constants and account for the polyclonal population of antibodies in the serum, GraphPad Prism 7.04 (GraphPad Software) was used to fit a 3-component exponential decay to the dissociation curves:

Y=Y1*exp(−K1*X)+(2*exp(−K2*X))+((Y3)*exp(−K3*X))

The initial values used for the model were:

Y1=0.25,Y2=0.25,Y3=0.5

K1=0.1,K2=0.001,K3=0.001

The component with the highest fraction and lowest dissociation rate was used for comparing the experimental groups.

In vivo passive transfer influenza challenge model: Serum was collected at day 56 after vaccination with A/Brisbane/59/2007(H1N1) hemagglutinin delivered in (i) TLR 7/8 NP-based 2:10 hydrogels, (ii) MF59, or (iii) Alum. Control serum was collected from a naïve mouse. Serum from 5 mice per group was pooled and antibodies were isolated as follows: First, a Zeba Spin Desalting Column (Thermo Scientific, 89882) was used to buffer exchange into Melon Gel Buffer, then IgG antibodies were purified using a Melon Gel IgG Spin Purification Kit (Thermo Scientific, 45206), and lastly the solution was buffer exchanged into PBS using the Zeba Spin Desalting Column. The columns were used following the manufacturer's guidelines. A mixture of the purified serum and PR8 virus was incubated at 37° C. for 30 minutes before nasal inoculation into C57/BL6 mice. The nasal inoculation contained 4 μL of purified serum, 2,000 PFU of PR8 virus, and PBS to a total volume of 20 μL. Mice were weighed daily to monitor morbidity and animals that exceeded 25% weight loss were euthanized.

### REFERENCES

1 Irvine, D. J., Swartz, M. A. & Szeto, G. L. Engineering synthetic vaccines using cues from natural immunity. Nat. Mater. 12, 978-990, (2013).
2 Luo, W., Weisel, F. & Shlomchik, M. J. B Cell Receptor and CD40 Signaling Are Rewired for Synergistic Induction of the c-Myc Transcription Factor in Germinal Center B Cells. Immunity 48, 313-326 e315, (2018).
3 Shlomchik, M. J. & Weisel, F. Germinal center selection and the development of memory B and plasma cells. Immunol. Rev. 247, 52-63, (2012).
4 Cirelli, K. M. & Crotty. S. Germinal center enhancement by extended antigen availability. Curr. Opin. Immunol. 47, 64-69. (2017).
5 Cirelli, K. M. et al. Slow Delivery Immunization Enhances HIV Neutralizing Antibody and Germinal Center Responses via Modulation of Immunodominance. Cell 177, 1153-1171 e1128, (2019).
6 Boopathy, A. V. et al. Enhancing humoral immunity via sustained-release implantable microneedle patch vaccination. Proceedings of the National Academy of Sciences USA 116, 16473-16478, (2019).
7 Zhang, J. et al. Stabilization of vaccines and antibiotics in silk and eliminating the cold chain. P. Natl. Acad. Sci. U.S.A. 109, 11981-11986, (2012).
8 de Veer, M., Kemp, J., Chatelier, J., Elhay, M. J. & Meeusen, E. N. The kinetics of soluble and particulate antigen trafficking in the afferent lymph, and its modulation by aluminum-based adjuvant. Vaccine 28, 6597-6602, (2010).
9 Lofano, G. et al. Oil-in-Water Emulsion MF59 Increases Germinal Center B Cell Differentiation and Persistence in Response to Vaccination. J. Immunol. 195, 1617-1627, (2015).
10 Cantisani, R. et al. Vaccine Adjuvant MF59 Promotes Retention of Unprocessed Antigen in Lymph Node Macrophage Compartments and Follicular Dendritic Cells. J. Immunol. 194, 1717-1725, (2015).
11 Meyer, S. et al. AIRE-Deficient Patients Harbor Unique High-Affinity Disease-Ameliorating Autoantibodies. Cell 166, 582-595, (2016).
12 Tam, H. H. et al. Sustained antigen availability during germinal center initiation enhances antibody responses to vaccination. P. Natl. Acad. Sci. U.S.A. 113, E6639-E6648, (2016).
13 Henriksen-Lacey, M. et al. Liposomal cationic charge and antigen adsorption are important properties for the efficient deposition of antigen at the injection site and ability of the vaccine to induce a CMI response. J. Control. Release 145, 102-108, (2010).
14 DeMuth, P. C., Min, Y., Irvine, D. J. & Hammond, P. T. Implantable silk composite microneedles for programmable vaccine release kinetics and enhanced immunogenicity in transcutaneous immunization. Adv. Healthc. Mater. 3, 47-58, (2014).
15 Moyer, T. J., Zmolek, A. C. & Irvine, D. J. Beyond antigens and adjuvants: formulating future vaccines. J. Clin. Invest. 126, 799-808, (2016).
16 Ali, O. A. et al. Identification of immune factors regulating antitumor immunity using polymeric vaccines with multiple adjuvants. Cancer Res. 74, 1670-1681. (2014).
17 Johansen, P. et al. Antigen kinetics determines immune reactivity. P. Natl. Acad Sci. USA. 105, 5189-5194, (2008).
18 Kemp, J. M. et al. Continuous antigen delivery from controlled release implants induces significant and anamnestic immune responses. Vaccine 20, 1089-1098, (2002).
19 Spiers, I. D., Eyles, J. E., Baillie, L. W., Williamson, E. D. & Alpar, H. O. Biodegradable microparticles with different release profiles: effect on the immune response after a single administration via intranasal and intramuscular routes. J. Pharm. Pharmacol. 52, 1195-1201, (2000).
20 Thomasin, C., Corradin, G., Men, Y., Merkle, H. P. & Gander, B. Tetanus toxoid and synthetic malaria antigen containing poly(lactide)/poly(lactide-co-glycolide) microspheres: importance of polymer degradation and antigen release for immune response. J. Control. Release 41, 131-145, (1996).
21 Preis, I. & Langer, R. S. A single-step immunization by sustained antigen release. J. Immunol. Methods 28, 193-197, (1979).
22 DeMuth. P. C., Garcia-Beltran, W. F., Ai-Ling, M. L., Hammond, P. T. & Irvine, D. J. Composite Dissolving Microneedles for Coordinated Control of Antigen and Adjuvant Delivery Kinetics in Transcutaneous Vaccination. Adv. Funct. Mater. 23, 161-172, (2013).
23 Pagels, R. F. & Prud'homme, R. K. Polymeric nanoparticles and microparticles for the delivery of peptides, biologics, and soluble therapeutics. J. Control. Release 219, 519-535, (2015).
24 Li, J. & Mooney, D. J. Designing hydrogels for controlled drug delivery. Nat. Rev. Mater. 1, (2016).
25 Appel, E. A., del Barrio, J., Loh, X. J. & Scherman, O. A. Supramolecular Polymeric Hydrogels. Chem. Soc. Rev. 41, 6195-6214, (2012).
26 Mann, J. L., Yu, A. C., Agmon, G. & Appel, E. A. Supramolecular polymeric biomaterials. Biomater. Sci. 6, 10-37. (2017).
27 Stapleton, L. M. et al. Use of a supramolecular polymeric hydrogel as an effective post-operative pericardial adhesion barrier. Nat. Biomed. Eng. 3, 611-620, (2019).
28 Appel, E. A. et al. Self-assembled hydrogels utilizing polymer-nanoparticle interactions. Nat. Commun. 6, 6295, (2015).
29 Appel, E. A. et al. Exploiting Electrostatic Interactions in Polymer-Nanoparticle Hydrogels. ACS Macro Lett. 4, 848-852, (2015).
30 Lopez Hernandez, H., Grosskopf, A. K., Stapleton, L. M., Agmon, G. & Appel, E. A. Non-Newtonian Polymer-Nanoparticle Hydrogels Enhance Cell Viability during Injection. Macromol. Biosci. 19, e1800275, (2019).
31 Grosskopf, A. K. et al. Injectable Supramolecular Polymer-Nanoparticle Hydrogels Enhance Human Mesenchymal Stem Cell Delivery. Bioeng. Transl. Med. 5, e10147, (2020).
32 Yu, A. C. et al. Scalable manufacturing of biomimetic moldable hydrogels for industrial applications. P. Natl. Acad. Sci. U.S.A. 113, 14255-14260, (2016).
33 Maikawa, C. L. et al. Block copolymer composition drives function of self-assembled nanoparticles for delivery of small-molecule cargo. J Polym. Sci., Part A: Polym. Chem. 57, 1322-1332, (2019).
34 Murphy, K., Travers, P., Walport, M. & Janeway, C. Janeway's Immunobiology. 8th edn. (Garland Science, 2012).
35 Saltzman, W. M. Drug delivery: engineering principles for drug therapy. (Oxford University Press, 2001).
36 Hafner, A. M., Corthesy, B. & Merkle, H. P. Particulate formulations for the delivery of poly(I:C) as vaccine adjuvant. Adv. Drug. Deliv. Rev. 65, 1386-1399, (2013).
37 Longhi, M. P. et al. Dendritic cells require a systemic type I interferon response to mature and induce CD4+Th1 immunity with poly IC as adjuvant. J. Exp. Med. 206, 1589-1602, (2009).
38 Desmet, C. J. & Ishii, K. J. Nucleic acid sensing at the interface between innate and adaptive immunity in vaccination. Nat. Rev. Immunol. 12, 479-491, (2012).
39 Kumar, H., Koyama, S., Ishii, K. J., Kawai, T. & Akira, S. Cutting edge: cooperation of IPS-1- and TRIF-dependent pathways in poly IC-enhanced antibody production and cytotoxic T cell responses. J. Immunol. 180, 683-687, (2008).
Tang, S., Habicht, A., Li, S., Seiffert, S. & Olsen, B. D. Self-Diffusion of Associating Star-Shaped Polymers. Macromolecules 49, 5599-5608, (2016).
41 Tang, S., Wang, M. & Olsen, B. D. Anomalous self-diffusion and sticky Rouse dynamics in associative protein hydrogels. J. Am. Chem. Soc. 137, 3946-3957, (2015).
42 Croguennec, T., Renault, A., Beaufils, S., Dubois, J.-J. & Pezennec, S. Interfacial properties of heat-treated ovalbumin. J Colloid Interface Sci. 315, 627-636, (2007).
43 Hangartner, L., Zinkemagel, R. M. & Hengartner, H. Antiviral antibody responses: the two extremes of a wide spectrum. Nat. Rev. Immunol. 6, 231-243, (2006).
44 Merad, M., Sathe, P., Helft, J., Miller, J. & Mortha, A. The dendritic cell lineage: ontogeny and function of dendritic cells and their subsets in the steady state and the inflamed setting. Annu. Rev. Immunol. 31, 563-604, (2013).
Krishnaswamy, J. K. et al. Migratory CD11b(+) conventional dendritic cells induce T follicular helper cell-dependent antibody responses. Sci. Immunol. 2, (2017).
46 Vidarsson, G., Dekkers, G. & Rispens, T. IgG subclasses and allotypes: from structure to effector functions. Front. Immunol. 5, 520, (2014).
47 De Silva, N. S. & Klein, U. Dynamics of B cells in germinal centres. Nat. Rev. Immunol. 15, 137-148. (2015).
48 Kasturi, S. P. et al. Programming the magnitude and persistence of antibody responses with innate immunity. Nature 470, 543-U136, (2011).
49 Haynes, B. F., Kelsoe, G., Harrison, S. C. & Kepler, T. B. B-cell-lineage immunogen design in vaccine development with HIV-1 as a case study. Nat. Biotechnol. 30, 423-433. (2012).
50 West, A. P., Jr. et al. Structural insights on the role of antibodies in HIV-1 vaccine and therapy. Cell 156, 633-648, (2014).
51 Kim, J. et al. Injectable, spontaneously assembling, inorganic scaffolds modulate immune cells in vivo and increase vaccine efficacy. Nat. Biotechnol. 33, 64-72, (2015).
52 Axelrod, D., Koppel, D. E., Schlessinger, J., Elson, E. & Webb, W. W. Mobility measurement by analysis of fluorescence photobleaching recovery kinetics. Biophys. J. 16, 1055-1069, (1976).
53 Miller, C. C. The Stokes-Einstein Law for Diffusion in Solution. Proc. R. Soc. London. Ser. A Cont. Paper Math. Phys. Charact. 106, 724-749, (1924).
54 Axpe, E. et al. A Multiscale Model for Solute Diffusion in Hydrogels. Macromolecules 52, 6889-6897, (2019).
55 Brandl, F. et al. Hydrogel-based drug delivery systems: comparison of drug diffusivity and release kinetics. J. Control. Release 142, 221-228, (2010).
56 Axpe, E. et al. A Multiscale Model for Solute Diffusion in Hydrogels. Macromolecules 52, 6889-6897, (2019).
57 Cirelli, K. M. & Crotty, S. Germinal center enhancement by extended antigen availability. Curr Opin Immunol 47, 64-69, (2017).
58 Crotty, S. T follicular helper cell differentiation, function, and roles in disease. Immunity 41, 529-542, (2014).
59 Murphy, K., Travers, P., Walport, M. & Janeway, C. Janeway's Immunobiology. 8th edn, (Garland Science, 2012).
60 Tregoning, J. S., Russell, R. F. & Kinnear, E. Adjuvanted influenza vaccines. Hum. Vaccin. Immunother. 14, 550-564, (2018).
61 Cirelli, K. M. et al. Slow Delivery Immunization Enhances HIV Neutralizing Antibody and Germinal Center Responses via Modulation of Immunodominance. Cell 177, 1153-1171 e1128, (2019).
62 Axelrod, D., Koppel, D. E., Schlessinger. J., Elson, E. & Webb, W. W. Mobility measurement by analysis of fluorescence photobleaching recovery kinetics. Biophys. J 16, 1055-1069, (1976).
63 Tande, B. M., Wagner, N. J., Mackay, M. E., Hawker, C. J. & Jeong, M. Viscosimetric, Hydrodynamic, and Conformational Properties of Dendrimers and Dendrons. Macromolecules 34, 8580-8585, (2001).
64 Miller, C. C. The Stokes-Einstein Law for Diffusion in Solution. Proc. R. Soc. London, Ser. A Cont. Paper Math. Phys. Charact. 106, 724-749, (1924).
65 Brandl, F. et al. Hydrogel-based drug delivery systems: comparison of drug diffusivity and release kinetics. J. Control. Release 142, 221-228, (2010).
66 Shukla. N. M. et al. Syntheses of fluorescent imidazoquinoline conjugates as probes of Toll-like receptor 7. Bioorg Med Chem Lett 20, 6384-6386, (2010).
67 Kandalkar, S. R et al. Highly efficient one-pot amination of carboxylate-substituted nitrogen-containing heteroaryl chlorides via Staudinger reaction. Tetrahedron Letters 54, 414-418, (2013).

### Example 3. Development of Nanoparticle-Conjugated TLR9 Agonist Adjuvants to Improve the Potency and Durability of COVID-19 Vaccines

### Introduction

Vaccines are among the most effective medical advancements in history to prevent and control infectious diseases. The eradication of smallpox, near eradication of poliomyelitis, and vast decreases in diphtheria, measles, and rubella are testaments to the ability of vaccines to mitigate disease burden worldwide^{1,2}. It is estimated that vaccines save 2.5 million lives worldwide per year³. However, there are an abundance of infectious diseases that still do not have effective vaccines; for example, influenza, which still kills roughly 500,000 people annually worldwide despite the yearly flu vaccines. Furthermore, new viral strains continue to emerge, threatening our society and challenging our humanity. The most notable current example is the ongoing pandemic caused by severe acute respiratory syndrome coronavirus 2 (SARS-CoV-2) that emerged at the end of 2019. To date, while this is being written, more than 60 million cases and 1.4 million deaths have been confirmed; marking the COVID-19 pandemic as the most serious health crisis of modern times. The COVID-19 pandemic highlights the increasing threat of new pandemic strains of viruses, and further motivates the need for continued improvement of vaccines technologies.

Different types of vaccines, such as inactivated viruses, live-attenuated viruses and subunit vaccines⁴ are commonly used in the clinic. The subunit vaccine approach offers an opportunity for more precise vaccine design, improved safety, stability, and inexpensive and scalable manufacturing capabilities, as well as less extreme storage conditions⁵. Subunit vaccines contain specific microbial antigens, which directs the antibody response to a specific foreign substance, along with one or more immune stimulating molecule(s), which are commonly referred to as adjuvants. Since subunit vaccines, such as the spike protein in COVID-19 vaccines, elicit weaker immune responses, adjuvants are needed to promote vaccine efficacy and the body's immune response to a pathogen⁶. As subunit vaccines become more popular, there is a growing need for new approaches to augment the adjuvant potency and guide the immune system to enhance the quality and durability of immune responses.

Adjuvants trigger various pathways of innate immune cell activation, such as pattern recognition receptor (PRR) signaling on innate immune cells. Currently, a handful of adjuvants that have been used in the clinic include aluminum salt-based adjuvants (Alum), squalene-based oil-in-water emulsions such as MF59 and AS03, and toll-like receptor agonists (TLRa) like the synthetic oligodeoxynucleotide CpG ODN (cytidine-phosphate-guanosine oligodeoxynucleotide) 1018. CpG 1018, specifically, is the only clinically approved version of CpG, is included in the Hepatitis B vaccine Heplisav B (2017), has been shown to increase the immune response by strongly activating B cells⁷ and is therefore currently being tested in phase I clinical trials with numerous SARS-CoV-2 vaccine candidates.

Synthetic CpG motifs are agonists for TLR9, mimic the activity of naturally occurring motifs found in bacterial DNA and are commonly used as immunostimulatory adjuvants to increase vaccine immunogenicity. Specifically. CpG triggers intracellular signaling leading to activation of macrophages, dendritic cells (DCs) and B-cells, as well as the production of chemokines and cytokines, enhancing innate and adaptive immune responses. CpGs are composed of short synthetic single-stranded DNA molecules of approximately 20 base pairs, contain unmethylated CpG dinucleotides in a specific sequence and have a phosphorothioated backbone to allow for increased stability. Three major classes of CpGs exist, including Class A. Class B, and Class C, and they differ based on their distinct biological activity and structural characteristics. Class B (CpG-B) strongly activates B cells and TLR9-dependent nuclear factor kappa-B (NF-kB) signaling pathways but weakly stimulates interferon-alpha (IFN-α) secretion, whereas Class C (CpG-C) both stimulates B cells and induces IFN-α production⁸. The biggest enhancer of the humoral response is CpG-B, which has been found to be a highly effective vaccine adjuvant inducing Th1 polarized responses⁸⁻¹⁰. Numerous studies have demonstrated innate immune defenses being activated by CpG-B¹¹⁻¹³ but fewer studies have been reported with CpG-C¹⁴⁻¹⁵.

To maximize the activation of antigen presenting cells (APCs) and other innate immune cells while minimizing systemic toxicities^{16,17}, significant efforts have focused on localizing TLR agonists, among other adjuvants, to the injection site and lymph nodes (LNs) using nanoparticles (NPs). Polymer-based NPs have been heavily studied due to their modularity, ease of production, scalability and biocompatibility¹⁶⁻²⁰. Furthermore, NPs have been widely employed in drug delivery applications²¹⁻²⁶. These advantages make NPs a powerful design platform to overcome the challenges associated with conventional delivery methods to enhance the spatiotemporal responses of vaccines²⁷. Precise control of the NPs size^{28,29,30,31-33} enables efficient targeting to LNs without requiring specific cell targeting ligands. NPs between 20 and 100 nm efficiently drain through the lymphatic system into the LNs^{28,29,34} where they are directly taken up by LN-resident APCs³⁵. Recent reports also have discovered that covalently conjugating TLRa to polymer NPs, marks a significant increase in both antibody production against the antigen and on the induction of cytotoxic T-cells¹⁷. Similarly, other studies have leveraged particle technologies to increase the density of presented adjuvant molecules on their surface to improve the potency of the adjuvant response^{17,36-38}. Controlling adjuvant presentation is a useful tool because many TLRs cluster upon ligand binding and this clustering effect can enhance downstream signaling, leading to a more potent response³⁹. There is a growing body of work demonstrating that immune cells require precise spatial and temporal cues to drive specified responses, supporting the idea that spatiotemporal control of vaccines can have a profound effect on the magnitude and quality of the immune response⁴⁰⁻⁴⁹.

Recent proof-of-concept studies evaluating slow delivery, have shown that sustained release of an HIV vaccine through implantable osmotic pumps leads to greatly improved quality of vaccine responses, such as durable germinal center (GC) responses, high antibody titers, and development of better virus neutralization compared to standard bolus administration of the same vaccine⁴⁶. Our group recently demonstrated the use of easily injectable physically cross-linked Polymer-NanoParticle (PNP) hydrogels for prolonged co-delivery of subunit vaccine components can greatly improve the potency, durability and breadth of the elicited immune response³⁹. PNP hydrogels are composed of polymers physically and dynamically crosslinked by multivalent noncovalent interactions with the NPs^{43,50-54}. This newly developed hydrogel, which can be administered in a minimally invasive fashion, is capable of providing sustained release of cargo molecules over the course of weeks, while prolonging the GC reaction and improving the affinity of the antibodies by more than 1000-fold⁴³.

In the current study we sought to optimize the delivery of CpG, by developing a NP adjuvant platform via chemically conjugating a Class B (CpG-B) or a Class C (CpG-C) CpG adjuvant to polymeric poly(ethylene glycol)-b-poly(lactic acid) (PEG-b-PLA) NPs of approximately 50 nm in size, allowing for efficient, passive and direct transport to the LNs (FIG. 30A). We hypothesize that the chemical conjugation of CpG to the NPs will reduce morbidity and systemic cytokine production while controlling the pharmacokinetics and enhancing the vaccine immunogenicity for induction of antibodies compared to free CpG. Firstly, we showed that TLR9a (CpG) activity was not affected by the conjugation to the NPs construct and that indeed the presentation of potent innate activators on the NPs surface increases their immunogenic potential compared to the free TLR9a. Furthermore, by precisely tuning the CpG valency on the NPs surface (FIG. 30B) we were able to control the resulting potency of the elicited immune response in vitro. Moreover, the developed modular and easily modifiable adjuvant-NPs platform is compatible with the previously described PNP hydrogels and allowed for prolonged and sustained release of vaccine cargos (FIG. 30B). We then compared the adjuvanticity of the CpG NPs and of the CpG encapsulated PNP hydrogels (i.e., PNP hydrogels with encapsulated CpG-NPs in it) in vivo in a COVID-19 vaccine study using the SARS-CoV-2 spike protein as the antigen. As such, we demonstrate rapid, potent and consistent IgG responses of the CpG PNP hydrogel vaccines compared to bolus injections of the CpG NPs vaccines already at day 7. PNP hydrogels containing CpG-NPs drove an increased antibody titer production that lead to a durable APC activation signal and to more potent vaccine responses. Furthermore, at day 21 both CpG PNP hydrogel vaccines were the only vaccine candidates eliciting broadly neutralizing antibodies, not allowing for any viral entry in the cells and protecting them from infection. Overall, this study shows the facile design of a broadly implementable adjuvant NPs platform technology with improved drainage, adjuvanticity and with increased potency and durability towards COVID-19 vaccines.

### Vaccine Immunity

A potent vaccine response results in durable production of neutralizing antibodies (humoral immunity), and in the activation of specific T cells (cell-mediated immunity). Antibodies bind to antigens on the pathogen's surface and block them from infecting host cells by neutralizing the pathogen. Cellular immunity involves T cells that can directly kill infected cells, which in turn eliminates the pathogen.

The materials discussed in this work primarily influence APCs and downstream humoral immunity, so those mechanisms are the focus of this section. The immune system first interacts with a vaccine at the site of injection. The extent of local inflammation is controlled by both the site of administration and the presence of immune activating molecules called adjuvants in the vaccine. Innate immune cells such as macrophages and DCs immediately respond to these adjuvants by producing cytokines to recruit cells to the site of injection. After the initial inflammatory response at the site of injection, activated DCs and the vaccine components themselves travel via afferent lymph vessels to the draining LNs^{55,56}. LNs provide a site where cells that are present at low frequencies within the body come together with precise spatial organization to enhance the cell-cell interactions necessary for generating a robust immune response. Migratory DCs present antigen directly to T cells themselves in the LN^{57,58}. The migration of immune cells can be enhanced by the use of adjuvants^{59,60}. In addition to antigen-presenting migratory DCs, there are also LN-resident DCs which are constantly scanning the lymph to capture any soluble antigens that reach the LNs by passive diffusion. The vaccine response therefore benefits from promoting antigen presentation and migration of tissue residents DCs as well as increasing antigen trafficking to the LN.

The overall goal of activating the innate immune system is to initiate an adaptive immune response to the vaccine, whereby APCs must transport and present antigen to B cells. Antigen-specific B cells proliferate and can differentiate into short lived antibody producing cells within a few days. Alternatively, antigen activated B cells can migrate into the B cell follicles in the LN, where they form dynamic microenvironments called germinal centers (GCs). The GC has two anatomical compartments, the dark zone where B cells proliferate and undergo somatic hypermutation (SHM) and the light zone where antigen-driven selection favors higher-affinity B cells⁶¹. It is very important for vaccines to initiate GC responses that are maintained for enough time to allow for many cycles of SHM to improve both, the breadth and the affinity of antibody responses. Finally, after the immune response is mounted, the circulation of antibodies in the blood allows them to reach infected tissues and protect the entire body.

### Materials and Methods

Poly(ethylene glycol)methyl ether 5000 Da (PEG), Poly(ethylene glycol)α-hydroxy-ω-azido terminated 5000 Da (N₃-PEG-OH), 3,6-Dimethyl-1,4-dioxane-2,5-dione (Lactide), 1,8-diazabicyclo(5.4.0)undec-7-ene (DBU, 98%), (Hydroxypropyl)methyl cellulose (HPMC, meets USP testing specifications), N,N-Diisopropylethylamine (Hunig's base), N-methyl-2-pyrrolidone (NMP), 1-dodecyl isocyanate (99%), mini Quick Spin Oligo columns (Sephadex G-25 Superfine packing material), Sepharose CL-6B crosslinked, bovine serum albumin (BSA), were purchased from Sigma-Aldrich, U.S. CpG-B 1826 (5′-TCCATGACGTTCCTGACGTT-3′) and CpG-C 2395 (5′-TCGTCGTTTTCGGCGCGCGCCG-3′) oligonucleotides, Aluminum hydroxide gel (Alhydrogel adjuvant 2%), zeocin were purchased from InvivoGen. Amino CpG-B 1826 (5′Amino Modifier C₆, 5′-NH₂-TCCATGACGTTCCTGACGTT-3′), amino CpG-C 2395 (5′Amino Modifier C₆, 5′-NH₂-TCGTCGTTTTCGGCGCGCGCCG-3′) were purchased from Integrated DNA Technologies (IDT); in order to allow for a direct comparison between the modified and unmodified CpG classes, unmodified CpG-B 1826 and CpG-C 2395 were also purchased from Integrated DNA Technologies. Dibenzocyclooctyne-PEG4-N-hydroxysuccinimidyl ester (DBCO-PEG4-NHS ester) and Alexa Fluor 647 DBCO (AFDye 647 DBCO) were purchased from Click Chemistry Tools. Dulbecco's modified Eagle's medium (DMEM, Gibco), phosphate buffered saline (PBS pH 7.4, Gibco) and Invitrogen E-Gel EX Agarose Gels 4% were purchased from Thermo Fisher Scientific. Heat Inactivated fetal bovine serum (HI-FBS) was purchased from Atlanta Biologicals. IFN-α cytokine enzyme-linked immunosorbent assay (ELISA) kit was purchased from PBL Assay Science, and the TNF-α cytokine ELISA kits was purchased from R&D Systems (Fisher Scientific). Goat anti-mouse IgG Fc secondary antibody (A16084) HRP (Horseradish peroxidase) was purchased from Invitrogen. 3,3′,5,5′-Tetramethylbenzidine (TMB) ELISA Substrate, high sensitivity was acquired from Abcam. HIS Lite Cy3 Bis NTA-Ni Complex was purchased from AAT Bioquest. Unless otherwise stated, all chemicals were used as received without further purification. Deionized (DI) water (Milli-Q, MilliporeSigma, 18 MΩ) was used for all the experiments. In order to remove any traces of water, all glassware and teflon-coated magnetic stir bars used for the polymer synthesis were dried overnight in the oven. Nuclear magnetic resonance (NMR) spectra of the polymeric structures were obtained on an Inova NMR spectrometer (¹H: 600 MHz, ¹³C: 150 MHz) with a Varian Inova console and using the VNMRJ4.2 software. Chemical shifts (δ) are given in parts per million (ppm) and peak multiplicity is reported as follow: s=singlet and m=multiplet.

PEG-PLA synthesis. PEG-PLA was prepared as previously reported⁵¹. Prior to use, the commercial lactide was recrystallized in ethyl acetate and DCM was dried via cryo distillation. Under inert atmosphere (N₂), PEG-methyl ether (5 kDa, 0.25 g, 4.1 mmol) and DBU (15 μL, 0.1 mmol) were dissolved in 1 mL of anhydrous DCM. Lactide (1.0 g. 6.9 mmol) was dissolved under N₂ in 3 mL of anhydrous DCM. The lactide solution was then quickly added to the PEG/DBU mixture and was allowed to polymerize for 8 min. The reaction was then quenched with an acetic acid solution and the product precipitated into a 1:1 mixture of ethyl ether and hexanes solution mixture, collected by centrifugation and dried under vacuum. ¹H NMR (600 MHz, CDCl₃, δ in ppm in ppm): 1.48-1.62 (m, 3H, CH₃ PLA), 3.64 (s, 4H, CH₂ PEG), 5.1-5.24 (m, 1H, CH, PLA).

Azide PEG-PLA synthesis. Azide-PEG-PLA was synthesized similarly to PEG-PLA. Prior to use, DCM was dried using 3-4 Å molecular sieves and N₃-PEG-OH was dried under vacuum overnight. Under inert atmosphere a solution of N₃-PEG-OH (0.5 g, 5 kDa, 100 μmol) and DBU (30 μL, 30 mg, 9.29 mmol) in anhydrous DCM (1 mL) was rapidly added to a solution of lactide (2.0 g 13.9 mmol) in anhydrous DCM (10 mL) and stirred for 8 min. The reaction mixture was quenched with an acetic acid solution, precipitated by an ethyl ether:hexane (1:1) mixture, centrifuged and dried under vacuum overnight. ¹H NMR (600 MHz, CDCl₃, δ in ppm): 1.48-1.62 (m, 3H, CH₃ PLA), 3.64 (s, 4H, CH₂ PEG), 5.1-5.24 (m, 1H, CH, PLA). ¹³C NMR (150 MHz, DMSO-d6, δ in ppm): 16.4 (C-B, PLA), 68.6 (C-C, PLA), 69.8 (C-A. PEG), 169.2 (C-D, PLA).

Synthesis of DBCO-CpG intermediate. DBCO-PEG-NHS ester (3.78 mg, 5.8 μmol) was dissolved in DMSO (40 μL). The solution was then diluted with PBS in order to reach a final concentration of 10 mM. The Amino-CpG-B and Amino-CpG-C (0.58 μmol) were then reacted with the DBCO-PEG4-NHS ester solution for 4 h at RT. The solution was then directly purified by size-exclusion in PBS, using a Sephadex G-25 Superfine (mini Quick Spin Oligo) column and stored at −20° C.

NPs synthesis and conjugation. PEG-PLA NPs were synthesized as previously described^{43,62}. A 1 mL solution of PEG-PLA in 75:25 AcCN:DMSO (50 mg/mL) was added dropwise to 10 mL of DI water stirring at 600 rpm. After NPs precipitation the particles solution was purified in centrifugal filters (Amicon Ultra, MWCO: 10 kDa) at 4500 RCF for 1 h and resuspended in PBS to reach a final concentration of 200 mg/mL. N₃-PEG-PLA NPs of different valencies (10%, 20%. 30% and 50%) were synthesized by premixing PEG-PLA and N₃-PEG-PLA polymer solutions at the correct volumetric ratios before precipitation. Purification and concentration of the CpG NPs were performed as above stated. For adjuvant conjugation the modified DBCO-CpG-B and DBCO-CpG-C, and N₃-PEG-PLA NPs were reacted via copper-free click chemistry for 12 h at room temperature. After reaction completion, CpG-B conjugated NPs and CpG-C conjugated NPs were purified by size-exclusion chromatography using a CL-6B matrix and stored in PBS at 4° C. Successful purification of the CpG-NPs from unreacted free CpG was confirmed via aqueous GPC and via agarose gel electrophoresis (4%). The CpG concentration on the NPs was determined through absorption calibration curves at 280 nm acquired using a Synergy H1 Microplate Reader (BioTek Instruments). An individual calibration curve for each NPs valency and TLR9 agonist class was recorded.

HPMC-C₁₂ synthesis. HPMC-C₁₂ was prepared according to a previously reported procedure⁵¹. HPMC (1.0 g) was dissolved in NMP by stirring at 80° C. for 1 h before removing the solution from the heat. Once cooled to RT. 1-dodexylisocynate (105 mg, 0.5 mmol) and Hunig's base, acting as the catalyst (˜3 drops) were dissolved in NMP (0.5 mL). This solution was then added dropwise to the reaction mixture, which was stirred at RT for 16 h. The resulting product solution was precipitated using acetone, decanted, redissolved in Milli-Q water (˜2 wt %) and dialyzed against water for 4 days. The polymer mixture was then lyophilized and reconstituted to a 60 mg/mL solution in sterile PBS.

Polymer characterization. After polymer synthesis relative molecular weights, number-average Mₙ, and weight-average M_{w}, molar mass, and polydispersity (PDI=M_{w}/Mₙ) were obtained from gel permeation chromatography (GPC) after passing through two size exclusion chromatography columns (Resolve Mixed Bed Low DVB, ID 7.8 mm, M_{w} range: 200-600,000 g/mol, Jordi Labs) in a mobile phase of DMF with 10 mM LiBr at 35° C. and a flow rate of 1.0 mL/min (Dionex Ultimate 3000 pump, degasser, and autosampler, Thermo Fisher Scientific). Before injection, samples at a concentration of 5 mg/mL were filtered through a 0.22 μm nylon membrane. The data acquired was analyzed using the Chromeleon7 GPC software. Nuclear magnetic resonance (NMR) spectra of the polymeric structures were obtained on an Inova NMR spectrometer (OH: 600 MHz, ¹³C: 150 MHz) with a Varian Inova console and using the VNMRJ4.2 software

NPs characterization. The hydrodynamic diameter of the NPs, before and after adjuvant conjugation, was characterized by dynamic light scattering (DLS) (DynaPro II plate reader, Wyatt Technology), and the NPs surface charge was measured with a Malvern Zetasizer Nano Zs. For the CpG-NPs, successful conjugation of the TLR9 agonist to the NPs was confirmed via aqueous GPC after passing through a SEC column [5000 to 5,000,000 g/mol]; Superose 6 Increase 10/300 GL (GE Healthcare) in a mobile phase of PBS containing 300 parts per million of sodium azide and at a flow rate of 0.75 mL/min (Dionex Ultimate 3000 pump, degasser, and autosampler, Thermo Fisher Scientific). Detection consisted of an Optilab T-rEX (Wyatt Technology Corporation) refractive index detector operating at 658 nm and a diode array detector operating at 280 nm (Dionex Ultimate 3000, Thermo Fischer Scientific). Before injection, samples at a concentration of 1 mg/mL were filtered through a 0.22 μm PVDF membrane. The data acquired was analyzed using the Chromeleon7 GPC software and the ASTRA software from Wyatt Technologies.

PNP and CpG-PNP Hydrogel formation. The CpG polymer-nanoparticle (CpG-PNP) hydrogels were formed at 2 wt % HPMC-C₁₂ and 10 wt % mixture of PEG-b-PLA and CpG-PEG-PLA NPs in PBS. The gels were prepared by mixing a 3:2:1 weight ratio of 6 wt % HPMC-C12 polymer solution, 20 wt % NPs solution, and PBS. Based on the desired adjuvant dosing, 30% CpG conjugated NPs were mixed with non-conjugated PEG-PLA NPs prior to hydrogel formation. The gels were formed by mixing the solutions using syringes connected through an elbow mixer.

Rheological Characterization of PNP gels. All rheological characterization was completed on a Discovery HR-2 Rheometer (TA Instruments). Measurements on the hydrogels were performed using a 20 mm serrated plate geometry at 25° C. and at 500 μm gap height. Dynamic oscillatory frequency sweeps were conducted at a constant 1% strain and angular frequencies from 0.1 to 100 rad/s. Amplitude sweeps were performed at a constant angular frequency of 10 rad/s from 0.5% to 10,000% strain. Flow sweep, steady shear experiments were performed at shear rates from 50 to 0.005 l/s, whereas stress-controlled flow sweep measurements were conducted at shear rates from 0.001 to 10 l/s. Step-shear experiments were performed by alternating between low shear rates (0.1 rad/s for 60 s) and high shear rates (10 rad/s for 30 s) for three full cycles. Yield stress values were extrapolated from stress-controlled flow sweep and amplitude sweep measurements.

FRAP Analysis. Fluorescence recovery after photobleaching (FRAP) was performed on the PNP hydrogel and on the CpG-PNP hydrogel formulations using a confocal LSM780 microscope. Each individual component of the hydrogel was labelled with a fluorescent component. NP-tethered AF647 (10 wt %), rhodamine-conjugated HPMC-C₁₂, His-tagged SARS-CoV-2 spike conjugated with HIS-Lite-Cy3 Bis NTA-Ni Complex (0.27 mg per mL of gel) were used to visualize diffusion of the vaccine cargo and the gel components. Samples were imaged using low intensity lasers to collect an initial level of fluorescence. Then a high intensity laser with an aperture of 25 μm was focused on the region of interest (ROI) for 10 s in order to bleach a circular area. Subsequently, fluorescence emission data was recorded for 4 min to create an exponential fluorescence recovery curve. For each sample, replicate measurements (n=2-5) were taken at multiple locations. The diffusion coefficient D was calculated according to the following equation⁶³:

$$D = γ D \frac{{ω}^{2}}{4 {τ}_{1 / 2}} \qquad ( 1 )$$

where the constant γ_{D}=τ_{1/2}/τ_{D}, with τ_{1/2} being the half-time recovery, to the characteristic diffusion time, both yielded by the ZEN software, and a) the radius of the bleached ROI. The diffusivity of the SARS-CoV-2 spike protein antigen in PBS was calculated using the Stokes-Einstein law equation for diffusion⁶⁴:

$$D = \frac{{k}_{B} T}{6 {πη R}_{H}} \qquad ( 2 )$$

with k_{B} being the Boltzmann's constant, T the temperature in Kelvin, η the solvent viscosity, and R_{H} the solute hydrodynamic radius. The hydrodynamic radius of the spike protein was measured via DLS to be R_{H}=12.2 nm, whereas qi for PBS was approximated to be 0.8872 mPa s at 25° C. The measured R_{H} agrees with the value published in literature and measured via Cryo-EM⁶⁵.

Vaccine formulation. The SARS-CoV-2 spike protein vaccines were injected subcutaneously either in form of a bolus injection or in form of a gel. For the bolus injections, the vaccine formulation contained a 10 μg antigen dose of spike S1+S2 ECD (R683A, R685A, F817P, A892P, A899P, A942P, K986P, V987P)-His Recombinant Protein (Sino Biological) and a 20 μg 30% CpG-NPs adjuvant dose; boosting was performed at day 21. For the CpG-PNP hydrogels, the dose was doubled containing 20 μg of antigen and 40 μg of CpG-NPs adjuvant; for the gel groups, boosting was not performed. Control groups were composed of non-conjugated PEG-PLA NPs, Alum (100 μg, Invivogen), and soluble CpG-B and CpG-C (20 μg. IDT) vaccines. Mouse blood was collected from the tail veins each week for 5 weeks. In order to analyze the early cytokine response, blood was collected at 0 h, at 3 h and 24 h from injection and stored at −80° C. These serum samples were analyzed for IFN-α and TNF-α levels and the concentrations were determined via enzyme-linked immunosorbent assay (ELISA) according to manufacturer's instructions and were calculated from standard curves. Absorbance was measured with a Synergy H1 microplate reader (BioTek Instruments) at 450 nm.

In vitro Raw-Blue reporter assay. The Raw-Blue (NF-kB-SEAP) reporter cell line (Invivogen, raw-sp) was used to evaluate the valency effect of TLR9 agonist conjugated to PEG-PLA NPs. The cells were cultured at 37° C. with 5% CO₂ in DMEM supplemented with L-glutamine (2 mM), D-glucose (4.5 g/L), 10% HI-FBS, and penicillin (100 U/mL)/streptonmcin (100 μg). Every other passage, zeocin (100 μg/mL) was added to the culture medium. Serial dilutions of soluble CpG-B and CpG-C and of the different CpG-B and CpG-C NPs formulations were added to a 96-well tissue culture treated plate to achieve final concentrations between 30 and 3.1 μg/mL of TLR9 agonist. Non-conjugated PEG-PLA NPs were used as a negative control. About 100,000 cells were added to each well in 180 μL of media and were incubated for 21 h at 37° C. in a CO₂ (5%) incubator. Manufacturer instructions were followed for SEAP quantification, and absorbance levels detected at 655 nm after 3 h incubation with QUANTI-Blue Solution (Invivogen). Nonlinear regression fits were found using the “Log(agonist) vs. response—EC₅₀” function in GraphPad Prism 8.4 software.

Animal studies. Six-to-seven weeks old female C57BL/6 (B6) mice were obtained from Charles River, housed in the animal facility at Stanford University and cared for according to Institutional Animal Care and Use guidelines. All animal studies were performed in accordance with the National Institutes of Health guidelines and the approval of Stanford Administrative Panel on Laboratory Animal Care. The day before vaccine administration mice were shaved in order to receive a subcutaneous injection of 150 μL of CpG-PNP gel or 100 μL of bolus on the right side of their backs. Mouse blood was collected from the tail veins each week for 5 weeks.

Antibody concentration. Serum Anti-spike IgG antibody endpoint titers were measured using an ELISA. Maxisorp plates (Thermofisher) were coated with SARS-CoV-2 spike protein at 2 μg/mL in PBS overnight at 4° C. and subsequently blocked with PBS containing 1 wt % BSA for 1 h at 25° C. Serum samples were serially diluted and incubated in the previously coated plates for 2 h at 25° C., and goat-anti-mouse IgG Fc-HRP (1:10,000) was added for 1 h at 25° C. Plates were developed with TMB substrate, the reaction stopped with 1 M HCl and the plates analyzed using a Synergy H1 microplate reader (BioTek Instruments) at 450 nm. End point titers were defined as the highest serum dilution at the one an optical density above 0.1 was detected.

SARS-CoV-2 Spike-pseudotyped Viral Neutralization Assay. Neutralization assays were conducted as previously described⁶⁶. Briefly, SARS-CoV-2 spike pseudotyped lentivirus was produced in HEK239T cells and cells seeded at six million cells the day prior to transfection. A five-plasmid system was used for viral production. Plasmids were added to filter-sterilized water and HEPES-buffered saline was added dropwise to reach a final volume of 1 mL. In order to form transfection complexes. CaCl₂ was added dropwise to the gently agitated solution. The transfection reactions were incubated for 20 min at RT and then added to plated cells. Virus-containing culture supernatants were harvested ˜72 hours after transfection via centrifugation and filtered through a 0.45 μm syringe filter. Viral stocks were stored at −80° C. For the neutralization assays, ACE2/HeLa cells were plated 1 to 2 days prior to infection and mouse serum was heat inactivated at 56° C. for 30 min prior to use. Mouse serum (1:50 dilution) and virus were diluted in cell culture medium and supplemented with polybrene at a final concentration of 5 μg/mL. Serum/virus solution at 1:50 were incubated at 37° C. for 1 h. After the 1 h incubation, the media was removed from the cells and incubated with the serum/virus solution at 37° C. for 48 h. After complete incubation, the cells were then lysed using BriteLite (Perkin Elmer) luciferase readout reagent, and luminescence was measured with a BioTek plate reader. Each plate was normalized by averaging the readout from the wells containing only the virus or only the cells.

Statistical Analysis. All results are expressed as mean±standard deviation (s.d). Comparison between two groups were conducted by a two-tailed Student's t-test. One-way ANOVA tests with a Tukey's multiple-comparisons test was used for comparison across multiple groups. Statistical analysis was performed using GraphPad Prism 8.4 (GraphPad Software). Statistical significance was considered as p<0.05.

### Results and Discussion

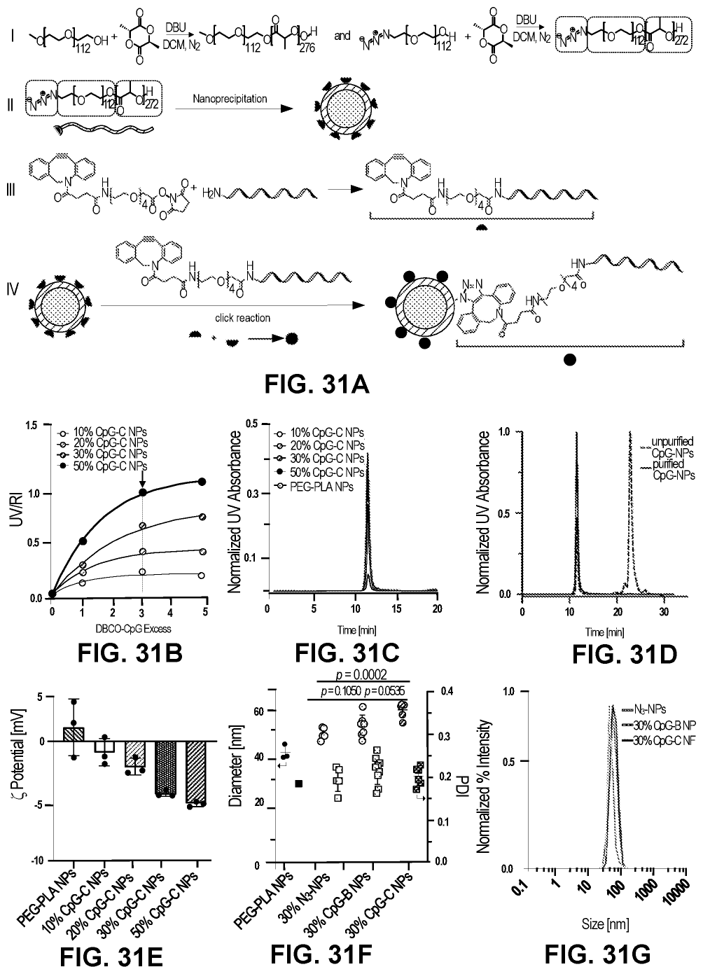

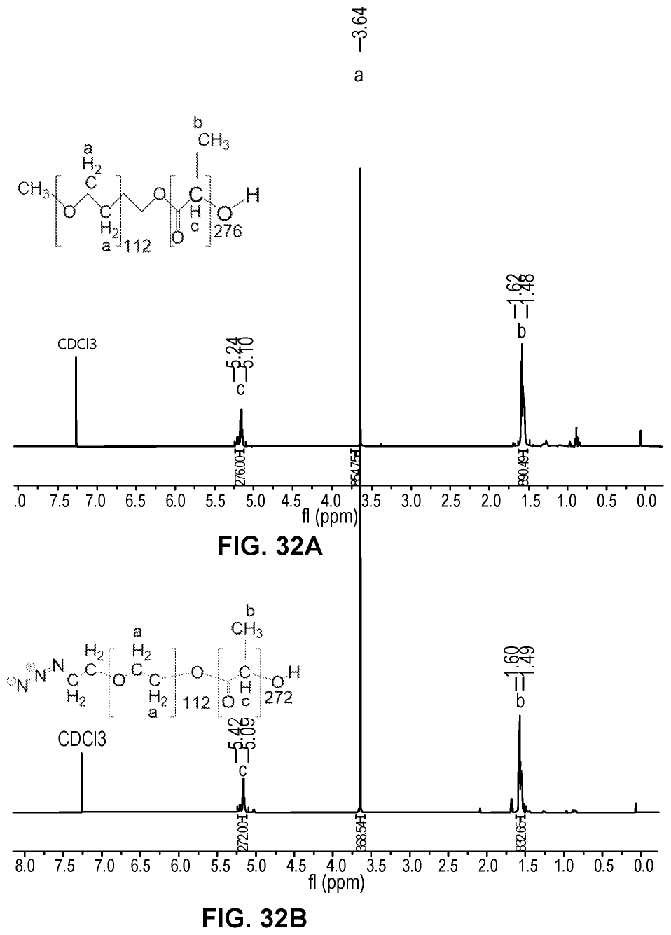

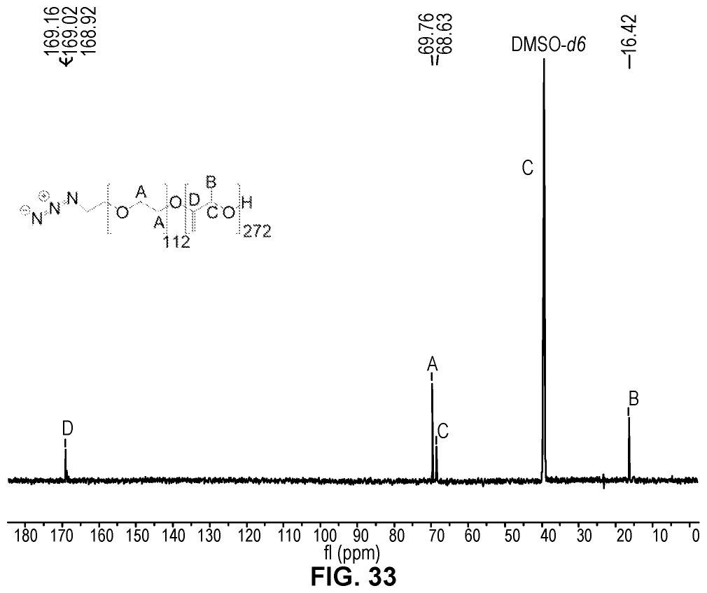

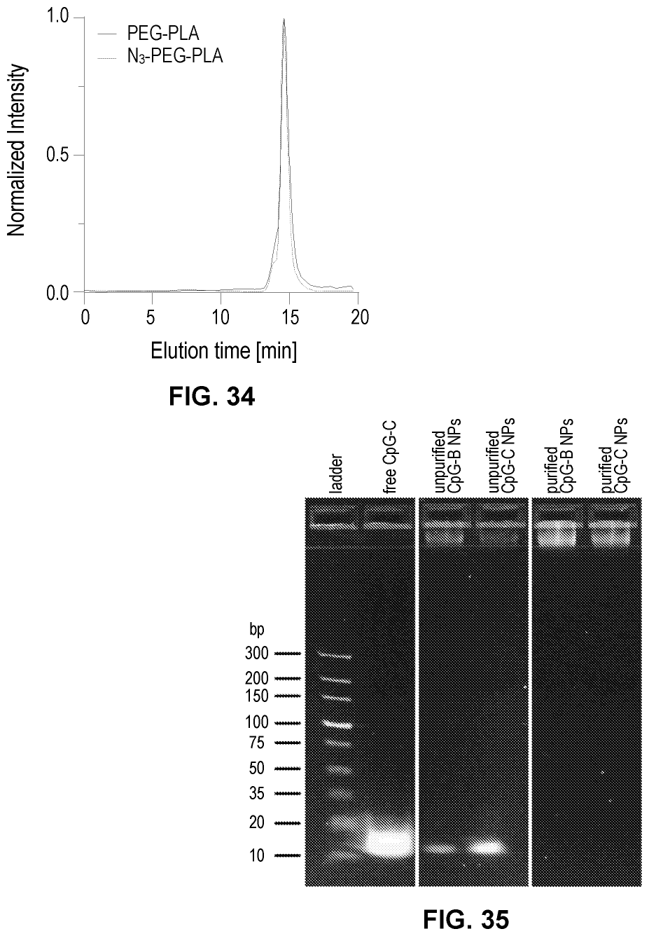

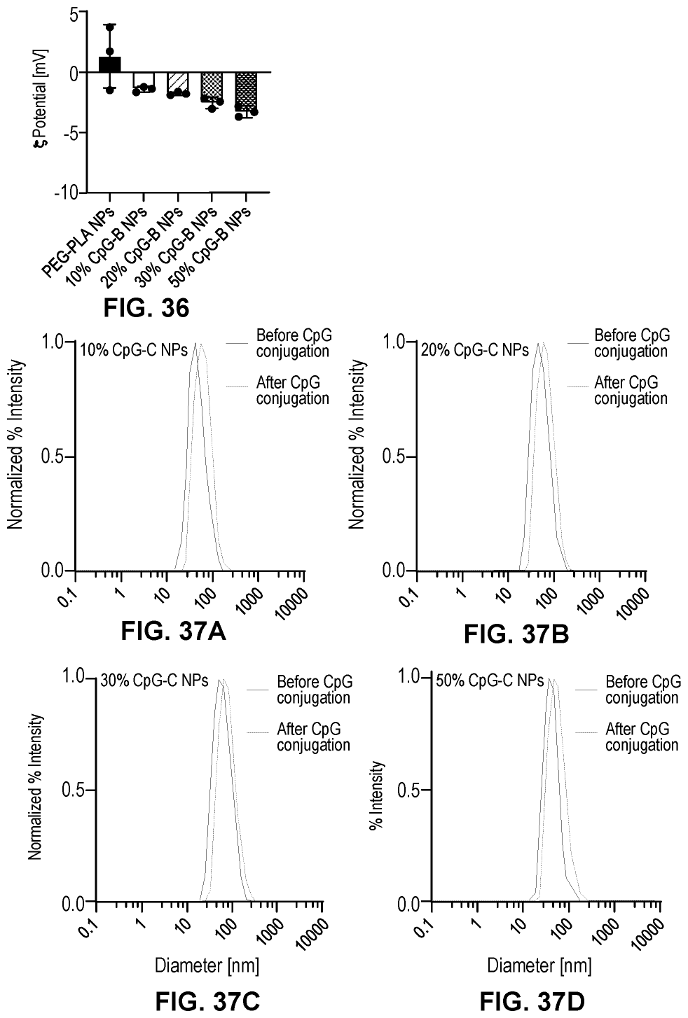

Chemical synthesis and purification of CpG NPs. In this study, we conjugated the TLR9 adjuvant CpG to PEG-PLA NPs in order to improve its stability, targeting to LNs and uptake by APCs. CpG is a strong inducer of Th1 immune responses that have proven important in clinical translation. We have previously described the synthesis of PEG-b-PLA) and azide-terminated PEG-PLA (N₃-PEG-b-PLA) block copolymers^{50,51,62,67} (FIG. 31A-1) for the fabrication of highly stable and monodisperse nanoparticles (N₃-NPs). These NPs form instantaneously when the respective polymer solutions are added dropwise to an antisolvent (FIG. 31A-II). Depending on the relative fractions of the two polymeric blocks, PEG-b-PLA copolymers self-assemble into different morphological structures, including core-shell type NPs^{25,26}. PEG-b-PLA was synthesized via organocatalytic ring-opening polymerization (ROP) using a PEG₅ₖ-methyl ether and lactide, or in the case of the azide-terminated PEG-b-PLA using N₃-PEG₅ₖ-OH and lactide (FIG. 31A-I). Each of the polymers prepared were characterized using nuclear magnetic resonance (NMR) spectroscopy and gel permeation chromatography (GPC). A molecular weight of 25 kDa, with a PEG block of 5 kDa and a PLA block of 20 kDa, was targeted for both polymers in order to allow for the hydrophobic PLA portions of the polymer chains to self-assemble to form a kinetically trapped entangled and stable core^{68,69}. ¹H-NMR and ¹³C-NMR spectroscopy were utilized to verify successful block co-polymer formation (FIGS. 32A, 32B, 33), whereas GPC was utilized to determine the M_{w}. Mₙ and PDIs of the polymers. Number average molecular weights were determined to be 24.9 kDa for the PEG-PLA (PD1=1.08) and 24.6 kDa for the N₃-PEG-PLA (PD1=1.1). Furthermore, co-elution of the clean polymer peaks is representative of comparable molecular weights and low polydispersity indices (PDI_{PEG-PLA}=1.08, PDI_{N3-PEG-PLA}=1.1) (FIG. 34).

The synthesized polymers were used as carriers to deliver CpG. CpG functionalized PEG-PLA NPs were synthesized via a two-step reaction: i) an NHS ester reaction to chemically link CpG to a cyclooctyne derivative functionality (FIG. 31A-III) a Copper free click reaction to conjugate the CpG to the NPs (FIG. 31A-IV). Firstly, the NHS ester-activated cyclooctyne, DBCO-PEG-4-NHS ester was reacted with NH₂-CpG to yield DBCO-CpG. DBCO-PEG-N₄-NHS ester was chosen as the reacting cyclooctyne-NHS ester in order to chemically activate the NH₂-CpG and also because of its fast kinetics, stability and enhanced solubility in water (PEG spacer). Secondly, the strain-promoted 1,3-dipolar cycloaddition of cyclooctynes and azides was used to tether DBCO-CpG to the N₃-PEG-b-PLA NPs and fabricate CpG functionalized NPs (CpG-NPs). This chemical strategy can be applied to conjugate different classes of CpG such as CpG-B and CpG-C, at varying densities onto the NPs surface by controlling the azide functionalization on the NP surface. Different densities of azide-functionalized NPs were formed by simply premixing PEG-PLA and N₃-PEG-PLA polymer solutions at the correct volumetric ratios before precipitation. Four different CpG-NP functionalization densities were fabricated: the increasing density of the CpG sequence on the NPs was denoted as 10% (low valency), 20% (low-medium valency), 30% (medium valency) and 50% (high valency) based on the loading of CpG. The higher the CpG valency, the higher the CpG density on the surface of the NPs.

CpG is a UV active molecule that absorbs UV light at a wavelength of λ=280 nm, therefore a successful conjugation to the NPs can be confirmed via an increase in UV/RI signal at the NPs elution time after passing through the SEC column of an aqueous GPC. A range of molar excesses of DBCO-CpG, from 1 to 5, were tested to optimize the conversion of the click reaction onto the differently functionalized NPs surface (FIG. 31B). An increase in conversion is directly proportional to its UV/RI ratio, when the conversion to excess curve reaches a plateau, it can be assumed that a quantitative amount of azide functionalities have reacted to CpG. High conversions between 88-97% were measured with three equivalents of DBCO-CpG. Moreover, increasing the quantity of reactants did not lead to a significant increase in terms of conversions. Based on these results, three equivalents of DBCO-CpG were chosen for each of the CpG-NP valencies. As expected, an increase in UV absorbance correlated with an increase in CpG density on the NPs surface (FIG. 31C). Because of the considerable size difference between CpG-NPs and DBCO-CpG (M_{w}=˜7.9 kDa), the SEC peaks were well separated from each other, with the CpG-NPs eluting at around 12 min and the free floating DBCO-CpG at around 23 min. After successful conjugation the free floating DBCO-CpG present in the reaction mixture was purified via SEC. To ensure complete removal of the unconjugated DBCO-CpG, the purified NPs were analyzed by GPC (FIG. 31D) and gel electrophoresis (FIG. 35).

Characterization of CpG NPs. Furthermore, it was important to confirm that the conjugation of TLR9 agonist to the NPs surface did not alter the NPs physical and colloidal properties. To this end, CpG-B and CpG-C NPs were characterized via surface zeta potential measurements (FIGS. 31E, 36) and via dynamic light scattering (DLS) (FIGS. 31F, 31G). In order to approximate the surface charge of the NPs under physiological conditions, the surface zeta potential measurements were conducted in diluted PBS. The negatively charged phosphorothioate backbone of CpG is responsible for a decrease in NPs surface charge with increased CpG conjugation. As the CpG density (higher CpG valency) on the NPs increased, the surface charge became more negative (FIG. 31E). Measuring the surface charge in diluted PBS results in screening of the surface charges through interactions with salt ions and therefore leads to smaller apparent surface charges than previously measured in water⁴³. A slightly negative surface charge, given by the hydroxyl and phosphorothioate groups on the surface of the NPs, is preferred as it electrostatically stabilizes the colloidal particles and prevents the formation of unwanted aggregation in vivo⁷⁰.

CpG NPs and plain NPs were characterized using DLS and were found to have hydrodynamic diameters of D_{H} of 50.0±3.1 nm (sd: 2.5, 30% N₃-NPs), 54.8±4.4 nm (sd: 5.8, 30% CpG-B NPs), 60.3±3.4 nm (sd: 3.2, 30% CpG-C NPs) (FIG. 31F). The DLS results show that the CpG conjugated NPs have a slightly larger diameter than the plain NPs. The relatively small standard deviations and narrow confidence intervals show that the following nanoprecipitation and chemical modification approach allows for NPs of different functionalization to be prepared in a consistent manner, largely without affecting the NPs hydrodynamic size (FIG. 31G, 37A-37D). Furthermore, based on previous literature we hypothesize that NPs of any size from 20-100 nm would promote LN trafficking^{28,29,34} while avoiding immediate partitioning of CpG into the blood stream and therefore reducing systemic toxicities.

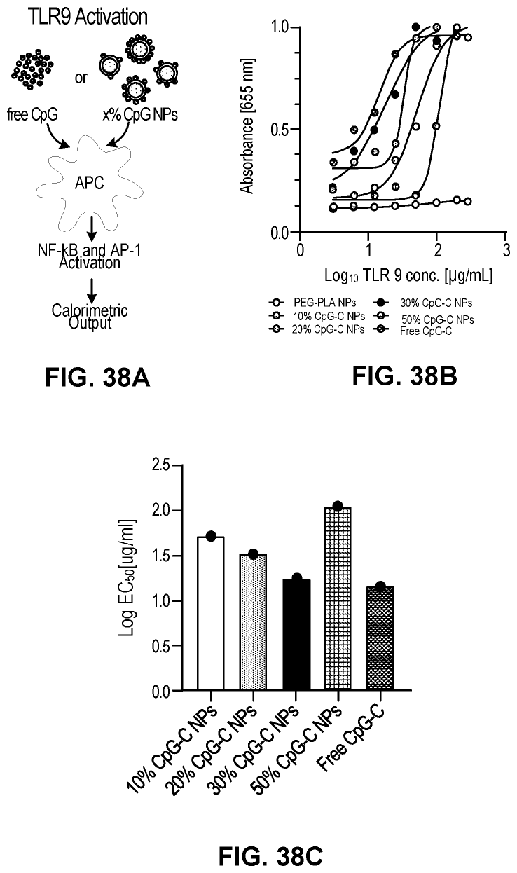

In vitro evaluation of CpG NPs potency. To ensure that the CpG conjugation to the NPs did not impair the biological activity and immunogenicity of the adjuvant, RAW-Blue transgenic mouse macrophage cells were used to quantify TLR9 activation. The cells were incubated with either CpG-C NPs, or soluble CpG-C. Activation of TLR9 by the CpG agonist induces signaling pathways leading to the activation of NF-kB and AP-1 (activator protein), and the subsequent secretion of secreted alkaline phosphatase (SEAP) which is easily detectable and measurable calorimetrically using QUANTI-Blue detection medium (FIG. 38A). Additionally, we conducted this in vitro assay to evaluate the impact of CpG-C valency on the NPs; specifically, valency's effect on the potency of innate immune cell activation. Different surface densities of TLR agonists on NPs have been shown to influence the magnitude and persistence of innate immune activation by leading to higher expression of co-stimulatory molecules¹⁷. We therefore hypothesized that there is an optimal density of CpG on the NPs at which the potency would be comparable to the free CpG species.

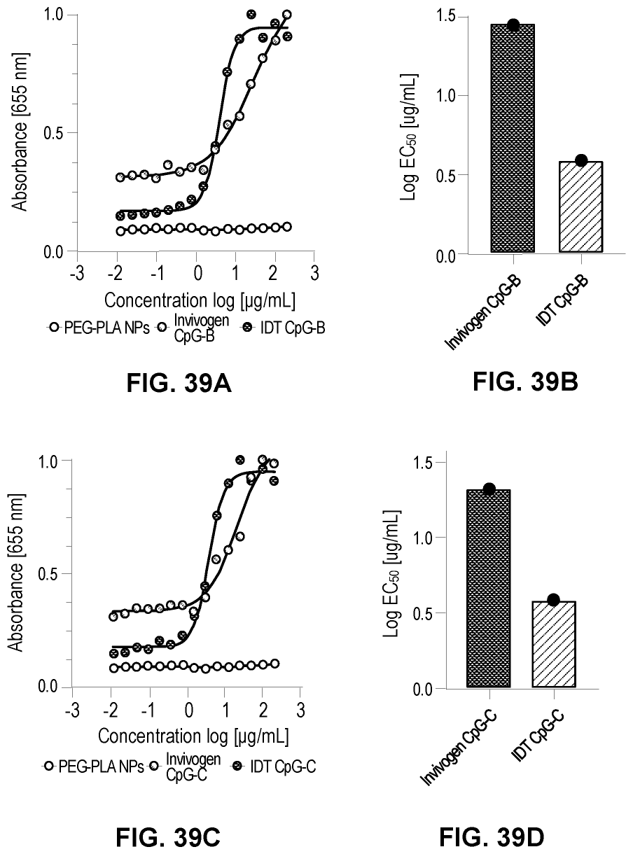

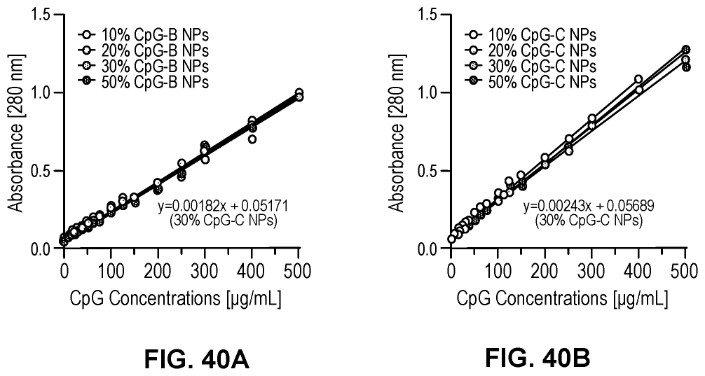

In these assays, Raw-Blue cells were incubated for 21 h with either the free form, or the NPs tethered form (10%, 20%, 30%, 50%) of CpG-C at a range of CpG concentrations (3.1-29 μg/mL) in order to generate concentration dependent activation curves (FIG. 38B). In order to have the most comparable free CpG control, both free CpG-B and CpG-C were purchased from IDT like the DBCO-CpG. CpG from IDT elicits similar responses to CpG from Invivogen (which is more commonly used) in our in vitro studies (FIGS. 39A-39D). In order to achieve the same administered dose of the active CpG molecule, the concentration of CpG-NPs required for a given dose will be inversely proportional to the valency. To ensure a correct and consistent dosing of CpG NPs throughout different experiments, we constructed a standard curve for both classes of CpG-NPs (FIGS. 40A, 40B) by measuring the absorbance of different concentration of CpG at 280 nm. It is important to denote that when dosing the CpG NPs, the concentrations are referred to the active CpG molecules tethered to the NPs and not the NPs concentration itself. Since we expected similar trends for Class B and Class C CpG NPs activation, we performed the in vitro study only on Class C CpG-NPs. From the dilution curves we then determined the EC₅₀ values (FIG. 38C), whereby EC₅₀ is the concentration of a drug that gives the half maximal response and is commonly used as the measure of a drug's potency. We observed that the CpG density influenced the resulting EC₅₀ values and therefore their potency. A lower EC₅₀ value is optimal as it demonstrates that a lower concentration is needed to reach the half maximum activation. An increase in CpG density, from the 10% to the 30% valency, resulted in increasingly lower EC₅₀ values; as a comparison, the 30% CpG-C NPs generated EC₅₀ values that were 66% lower (log EC₅₀=1.2 μg/mL) than the EC₅₀ value (log EC₅₀=1.7 ug/mL) of the 10% valency curve. Furthermore, the 30% CpG-C NPs performed best compared to all other valencies and behaved similarly to the free CpG-C (log EC₅₀=1.15). Interestingly, the 50% valency performed worse than all the other NPs, resulting in the highest EC₅₀ values (log EC₅₀=2.1 μg/mL), and therefore lowest activation. We hypothesize that the reduction in potency observed with the 50% CpG-C NPs was most likely due to the highly negatively charged surface, which by surpassing the critical threshold for charge density, decreased the association with cells and thereby reduced its potency.

In vitro, none of the tested NPs performed as well as the free CpG, demonstrating that conjugation of adjuvants to PEG-b-PLA NPs slightly decreases the potency of the molecule. However, in vitro cell assays do not take pharmacokinetics or biodistribution into account, and therefore are not always a good proxy for in vivo studies⁷¹. Based on these results, the 30% CpG valency NP was identified as the most promising CpG NPs adjuvant to promote high magnitude and persistent immune activation and was therefore tested in an in vivo vaccine study with SARS-CoV-2 as the antigen.

Together, these data demonstrate that we were able to synthesize pure and stable CpG NPs of different valencies, that were capable of activating TLR9 and showed similar potencies in vitro to free CpG. It has recently been shown, that the density and valency of antigen and adjuvant in vaccine design, dictates the magnitude and composition of the immune response⁶.

Furthermore, recent studies have highlighted the importance of vaccines in initiating GC responses that are maintained for long enough to improve the breadth and affinity of antibody responses^{46,72}. Sustained delivery technologies can promote long-lived GCs and therefore increase the magnitude and persistence of the humoral immune response. We have previously described the development of injectable PNP hydrogel materials that can encapsulate physiochemically diverse vaccine components such as antigens and adjuvants and can be tuned to provide sustained co-delivery over extended periods of time. Because of these characteristics, we expected that by loading and implementing our adjuvant NPs with improved potency into the slow and controlled release PNP hydrogels, we will further enhance the vaccine response.

Formation of CpG-PNP hydrogels and rheological characterization thereof. Physically crosslinked PNP hydrogels can be quickly and easily formed by mixing aqueous solutions of hydroxypropyl methylcellulose derivatives (HPMC-C₁₂) and biodegradable PEG-b-PLA NPs^{43,50,53,54} (FIG. 41A). After rapid mixture of the aqueous NPs and HPMC-C₁₂ using an elbow mixer (FIG. 41B), the HPMC derivative and the PEG-b-PLA NPs form multivalent, physically associated and dynamic interactions holding together the hydrogel structure. The multiplex interplay of non-covalent interactions provides the hydrogel with complex rheological behaviors ranging from viscoelasticity to plasticity, shear-thinning and thixotropy (FIG. 41C). These supramolecular hydrogels present solid-like properties when under static conditions and liquid-like behaviors under shear: this allows them to be injected but also to form solid depots (FIGS. 41B, 41C). For the sake of this study we chose to use a 2 wt % HPMC-C₁₂ and a 10 wt % NP formulation. Furthermore, the PNP hydrogels can easily be loaded with adjuvants and antigens within the hydrogel network⁴³. Adjuvant molecules such as free CpG can be loaded in the aqueous phase of the gel, but given their small molecular size and hydrophilicity, they rapidly diffuse out of the gel. In order to retain CpG over a long period of time in the gel, it is critical to use the above synthesized CpG NPs. We implement CpG-NPs in the gel to deliver the CpG adjuvant, making them an integral part of the polymer network, while contributing to the formation of non-covalent interactions with the polymer chains.

To ensure that the CpG-conjugation to the NPs did not influence the mechanical properties of the PNP gels, an extensive rheological characterization was performed where we compared, PNP hydrogels loaded with the equivalent of 20 ug of CpG-C in the form of CpG-C NPs (CpG-C PNP) to PNP hydrogels loaded with non-functionalized NPs (FIGS. 41D-41I). The rheological characterization was performed with class C CpG NPs. Previous experiment conducted in our lab proved that the molecular sequence of the CpG oligonucleotide does not significantly alter the effect on rheological properties (unpublished data). PNPs are complex fluids which possess viscoelastic, yielding, shear-thinning, and thixotropic behaviors. Here, we explored how each of these responses was affected by the introduction of CpG-C NPs.

In order to measure the hydrogel's viscoelastic response, frequency-dependent oscillatory shear experiments were conducted within the linear viscoelastic regime (LVER). The introduction of CpG-NPs did slightly alter the PNP's frequency response. Gels with (CpG PNP gels) and without (bare PNP gels) CpG-NPs exhibited solid-like properties within the explored frequency range (0.1 rad/s-100 rad/s) where the storage (G′) modulus was greater than the loss (G″) modulus. At a representative angular frequency of ω=10 rad/s the CpG-C PNP and the PNP gels exhibited storage moduli of 440 and 430 Pa, respectively. No crossover frequency was measured within this frequency range.

The yielding response of the hydrogels was measured with both dynamic amplitude sweeps and stress-controlled flow experiments. A dynamic amplitude sweep (FIG. 41E) was performed at a frequency of 10 rad/s: for both the CpG PNP and the PNP gel a yield stress of approximately 900 Pa was measured at the crossover point of G′ and G″. These results were consistent with the yield stress measured via stress-controlled flow sweep measurements. The response of the CpG PNP gel is shown in FIG. 41F, which shows the measured viscosity versus the imposed stress. The yield stress is the stress at which, for a very small increase in stress there is a significant decrease, of approximately 3 orders in magnitude, in viscosity, corresponding to approximately 940 Pa. Both yield stress measurements agreed and resulted in very high yield stresses (FIG. 41G). To compare the gel's abilities to shear-thin flow sweep measurements were performed. Both samples showed decreasing viscosities with increasing shear rates and similar shear-dependent viscosities (FIG. 41H). The viscosity of polymer solutions and physically crosslinked hydrogels adhere to this power law⁷³:

η=K\dot{γ}ⁿ⁻¹ (3)

with K being the consistency index, \dot{γ} the shear rate and n the shear thinning parameter. A shear thinning parameter below 1 is representative of a shear thinning fluid; a scaling exponent of −1 (n=0) is an evidence for a pre-yielding behavior. The flow portion of the viscosity vs. shear rate plot was fitted to the power law in order to find the shear thinning parameter. For the PNP and CpG-C PNP hydrogels n˜0.2 and n˜0 were extrapolated. From this fit we conclude that PNP hydrogels show dramatically shear thinning behaviors, whereas CpG-C PNP hydrogels show yielding. However, it has to be noted that the flow sweep measurement of the CpG-C PNP hydrogel has to be critically evaluated since the gel was partly ejected from the geometry.

The PNP hydrogels also display injectability, which is defined as the ability of the gel to flow at pressures that are clinically applied while injecting through a needle. To investigate the hydrogels' ability to self-heal after flow, step-shear experiments were performed by stepwise interchanging between low (0.1 l/s) and high (10 l/s) shear rates for 3 full cycles. At low shear rates the viscosity was of about 1000 Pa s, which decreased by 2 orders of magnitude to approximately 15 Pa s upon the application of elevated shear rates. The hydrogels are thixotropic, showing a considerable delay in restructuring their dynamic structure. This phenomenon is observable in the step-shear experiments where, once the shear is decreased to a steady-state value, the viscosity slowly increases to its equilibrium value (η˜1000 Pa s) while rebuilding its internal network (FIG. 41I). The inspected rheological properties are consistent with the presence of dynamic crosslinking motifs, where upon shearing a rupture in the physical crosslinks between NPs and polymers occurs.

The rheological characterization of these materials supports that the CpG-PNP hydrogels can be injected through high-gauge (e.g., 21-gauge) needles (FIG. 41B-iv) and maintain a robust gel structure after injection to allow for the formation of a local inflammatory niche^{43,74}.

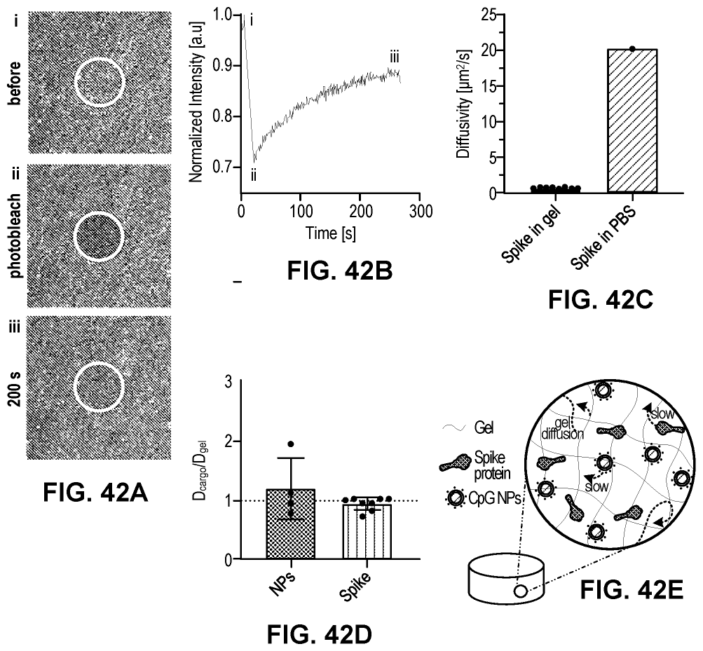

Vaccine cargo dynamics in PNP hydrogels. The different physiochemical properties of vaccine components pose a challenge for their controlled and sustained delivery. Vaccine components such as antigens and adjuvants can have extremely distinct polarities, molecular weights and hydrodynamic radii (R_{H}) influencing their diffusivity in the hydrogel network⁴³. CpG-PNP hydrogels are loaded with CpG tethered NPs (CpG NPs, R_{H}˜30 nm) and spike protein (R_{H}=12 nm. M_{w}=139 kDa). Because of its small molecular weight and hydrodynamic radius, we tethered the CpG nucleotide to the NPs. Unconjugated CpG (R_{H}˜4 nm⁷⁵, M_{w}˜7 kDa) is hypothesized to be smaller than the gels' mesh size⁶⁴ and therefore would rapidly diffuse out of the gel, not allowing for sustained delivery. In order to tune and characterize the dynamics of vaccine exposure expected in the PNP hydrogels, we performed fluorescence after photobleaching experiments (FRAP) (FIGS. 42A-42E). FRAP is a microscopy-based technique capable of determining and quantifying the kinetics of diffusion of fluorescently labelled molecules. We labeled the main gel components (e.g., both the NPs and the HPMC-C₁₂) through conjugation of a fluorescent dye and used a fluorescent polyhistidine nickel complex to label the spike antige. The diffusivity of each component was assessed in distinct experiments in order to isolate the individual diffusivity effects. A defined circular region (ROI) of the fluorescent gel was selectively photobleached by a high-intensity laser source (FIG. 42A). The mobile fluorescently tagged molecules passively diffused over time throughout the sample, enabling the intensity of the bleached area to recover by an exchange of bleached and unbleached fluorophores. The recovery was monitored over time until the point where uniform intensity was restored (FIG. 42B). From the fluorescent recovery curve, we determined the recovery half time inn, which was then used to calculate the gel diffusivities D of NPs, HPMC-C₁₂, and spike protein. FRAP measurements showed that the hydrogel network significantly reduced the cargo diffusivity of the spike protein by 97%, with a measured diffusivity of Dₛₚᵢₖₑ=0.64 μm²/s in the hydrogel compared to the calculated antigen's diffusivity in PBS of D=20.22 μm²/s (DLS, R_{H}=12 nm) (Eq. (2), FIG. 42C). Moreover, the self-diffusion of the PNP matrix D_{gel} was determined by measuring the diffusivity of the HPMC-C₁₂ within the hydrogel and amounted to D_{gel}=0.98 μm²/s.

In order to compare the diffusivity of the CpG-NPs (D_{NP}=0.68 μm²/s) and the spike protein in the gel (Dₛₚᵢₖₑ=0.64 μm²/s), to the self-diffusion of the PNP matrix, we calculated D_{cargo}/D_{gel} ratios close to 1 (FIG. 42D). Ratios around 1 are representative of cargo diffusion at rates close to the self-diffusivity of the hydrogel polymer matrix itself. This indicates that both cargoes, despite of their physiochemical differences, are largely immobilized by the hydrogel's network and are therefore only able to diffuse at a rate limited by the gel self-diffusivity thanks to the continuous rearrangement of the network bonds allowed by the dynamic nature of the physical network (FIG. 42E).

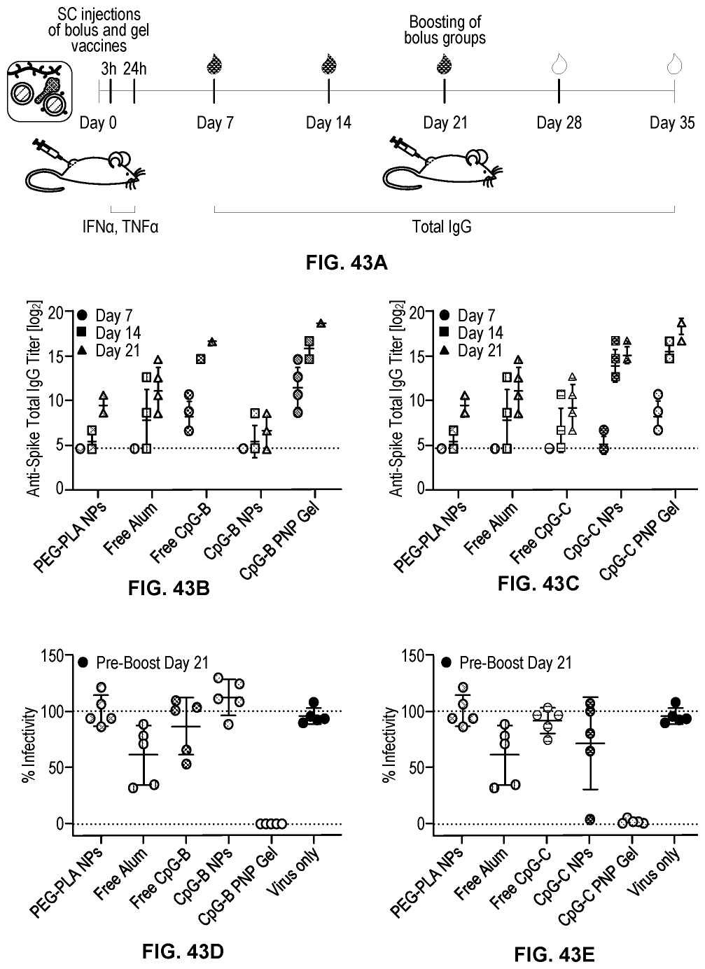

Humoral immune response to COVID-19 vaccine. In order to investigate if sustained delivery of newly developed CpG NPs would result in a more potent humoral immune response, we subcutaneously (s.c.) vaccinated mice against COVID-19 with spike protein and CpG delivered in different ways (i.e., CpG-NPs and CpG-PNP hydrogels). The spike protein (S) is currently one of the most promising antigens used in SARS-CoV-2 subunit vaccine research. The monomer has a molecular weight of 139 kDa and consists of two subunits, the S1 and S2, S1 contains a receptor binding domain (RBD) which recognizes and binds the cell surface receptor, whereas S2 contains basic elements required for membrane fusion. We therefore decided to fabricate different vaccines containing spike administered either with CpG in soluble form, CpG tethered to the NPs (CpG NPs) or CpG tethered to the NPs and encapsulated in a PNP gel (CpG PNP). For the two CpG NP groups, the CpG-B NPs and CpG-C NPs, mice were administered with 10 μg of spike antigen and 20 μg of CpG adjuvant. Furthermore, as a comparison we included a total of 4 control groups: 2 groups receiving spike adjuvanted with either soluble CpG-B, or CpG-C. 1 group receiving a vaccine adjuvanted with Alum (100 μg) as a clinically-relevant control, and 1 group receiving only the spike antigen with a small concentration of plain NPs added to it as a vehicle control. For the bolus injections, requiring a second dose of vaccination, s.c. priming was followed by the subsequent booster immunization at day 21 (FIG. 43A). Furthermore, it has become clear, that vaccination against COVID-19 will pose extensive manufacturing, distribution, logistics and administration challenges. These obstacles could be overcome by the generation of a vaccine that only requires a single dose.

As previously confirmed via FRAP measurements, the size of both, the monomeric spike protein and the CpG conjugated NPs are ideal for sustained co-delivery from the PNP hydrogel. Consequently, we hypothesized that the implementation of our newly developed CpG NPs loaded in the PNP hydrogel could considerably increase the potency of a COVID-19 vaccine, eliciting a broadly neutralizing antibody response after only one single immunization. In order to investigate this, we s.c administered 2 different CpG PNP hydrogels, a CpG-B PNP and a CpG-C PNP, with double the cargo dose of the bolus groups. We argue that administering a double dose of vaccine (20 μg of CpG and 40 μg of spike protein) would provide, over the course of the 35 days of study, the same total amount of vaccine as the bolus injections, however slowly releasing the antigen and adjuvant in a controlled manner over time. To achieve the 40 μg dose of CpG in the gels, the CpG NPs were mixed with standard PEG-b-PLA NPs.

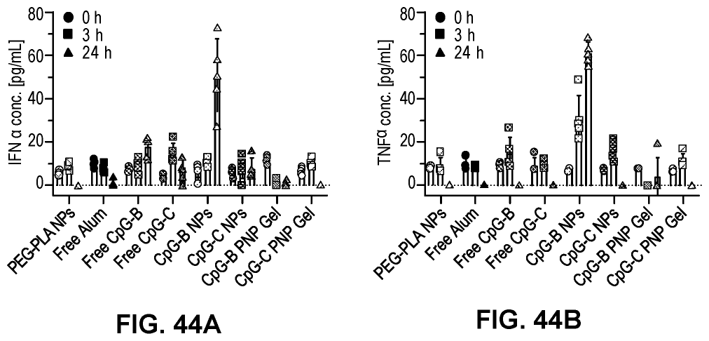

To ensure that the injected vaccines did not drive systemic cytokine responses that could pose a safety risk, we assessed the early inflammatory cytokines IFN-α (FIG. 44A) and TNF-α (FIG. 44B) at 3 h and 24 h after injection. No increased cytokine levels were detected (˜60 μg/mL) across the groups, including the two gel groups loaded with double the vaccine dose.

To assess the humoral response, mice serum was collected every week for 35 days and spike-specific immunoglobulin G (IgG) endpoint antibody titers were quantified every week. Evaluation of total IgG endpoint titers over the first 21 days (FIGS. 43B, 43C) indicated that both classes of CpG in the CpG PNP hydrogels, led to higher anti-spike IgG endpoint titers compared to the bolus administration of CpG NPs and of the soluble CpG. These observations may result from the formation of a local inflammatory niche that allows for infiltration of cells (FIGS. 30A, 30B) and for sustained co-delivery of the vaccine, whereas in case of the bolus injections we hypothesize that the passive drainage of CpG NPs to the LNs, results in activation of a strong downstream response. Furthermore, the hydrogels drove much more rapid switching to IgG from IgM, with high levels of IgG already detectable at day 7, when most of the other treatments had titers that were barely detectable. These observations are corroborated by other in vivo vaccine studies our group conducted in the past (unpublished data). It is also worth noting, that the gel based delivery of COVID-19 vaccines led to more consistent results within a given group, with significantly less error than for the bolus vaccine injections. In case of the CpG-B response (FIG. 43B), the soluble CpG-B vaccine showed a stronger response than the CpG-B NP, whereas for CpG-C (FIG. 43C), we observe the opposite trend, with the CpG-C NPs eliciting double the anti-spike IgG response compared to the soluble CpG-C. However, it is also worth noticing that the CpG-B PNP gels are eliciting antibody responses as strong as the CpG-C PNP gels, suggesting that the creation of a s.c. slow-release depot might be beneficial for the uptake of antigen and adjuvant. The nature of the observed response to the two classes of CpG, implies that different CpG classes activate different downstream pathways and therefore different cell populations. In order to corroborate this hypothesis, we plan to run a cell infiltration study, analyzing the different populations of cells present in the gels via flow cytometry.

Furthermore, on day 21, before boosting, we conduced neutralization assays (FIGS. 43C, 43D). To analyze the corresponding neutralizing titers, we used lentivirus pseudotyped with SARS-CoV-2 spike and determined the inhibition of the viral entry into HeLa cells overexpressing the angiotensin-converting enzyme 2 (ACE2) surface receptor. ACE2 serves as the entry point of SARS-CoV-2 into cells. The binding of the S1 subunit of SARS-CoV-2 to ACE2 on the surface of the cells results in the internalization of the virus allowing for its replication within the cells. As pseudotyped viruses are only able to replicate once and lack the virulent components of the full virus, neutralization with pseudotyped viruses allows for these assays to be conducted in BSL2 laboratories. The analysis of the immunized mice sera at a 1:50 dilution, revealed that the CpG PNP hydrogel vaccines showed notable and consistent neutralizing activity only after 21 days. Only the sera from mice injected with the CpG PNP hydrogels, from both CpG-B and CpG-C protected the cells from viral entry (0% infectivity), whereas all the other groups, allowed for at least 50% infectivity of the cells (FIGS. 43C, 43D). The neutralization assay shows that even though the levels of the bolus injection vaccines show relatively high anti-spike IgG antibody titers, not all the antibodies produced are non-neutralizing and so able to protect against an infection. This confirms that the combination of slow vaccine release and inflammatory niche, greatly enhances the formation of broadly neutralizing antibodies (nAbs).

The data gathered until day 21 is premature to make any conclusions about the final efficacy of the vaccines, however it already seems to appear that the prolonged co-delivery of both the antigen and the adjuvant is critical for eliciting strong and durable immune responses and for eliciting broadly neutralizing antibodies.

### Conclusions

In conclusion, we developed a novel adjuvant NPs platform where potent TLR9 agonists are presented on the surface of PEG-PLA NPs. These NPs allowed us to control adjuvant distribution on the NP surface while also exhibiting efficient transportation to the LNs due to their size. We also showed that the density of CpG presentation on the NP surface influenced the activation of TLR9, with 30% CpG NPs exhibiting similar potencies in vitro to free CpG. Additionally, in order to elicit a strong humoral response, improved spatiotemporal control over the vaccine delivery and distribution is needed. Therefore, we loaded our CpG-NPs in PNP hydrogels and showed through an extensive rheological characterization that the integration of CpG-NPs into the polymer network did not affect the overall mechanical properties of the PNP gels. The CpG PNP gels exhibited viscoelastic, thixotropic and shear thinning properties that allowed them to be injected and to maintain a robust solid-like structure after injection. Loading of the CpG PNP hydrogels with spike protein, as the antigen, and CpG-NPs, as the adjuvant, allowed both cargoes, despite of their physiochemical differences, to be largely immobilized by the hydrogel's network. Both vaccine components were therefore able to diffuse at similar rates out of the gel, allowing for a sustained and controlled release. In an in vivo COVID-19 vaccine study, we then showed that CpG PNP hydrogels were critical for eliciting strong immune responses. As such, we demonstrated rapid, more potent and consistent IgG responses of the CpG PNP hydrogel COVID-19 vaccines compared to bolus injections of the CpG NPs vaccines already at day 7. Furthermore, CpG PNP hydrogels led to durable APCs activation, which is necessary for the downstream humoral response, and increased antibody titers. Moreover, CpG PNP hydrogel vaccines were the only vaccine candidates in our study that elicited strong neutralizing antibody responses and protected cells from viral infection.

In sum, this study reports the development of a highly modular adjuvant NPs platform that provides for facile incorporation into PNP hydrogels for prolonged co-delivery with subunit antigens. We showed that these CpG-PNP hydrogels present a tunable platform for enhancing the humoral immune response to a COVID-19 subunit vaccine, contributing to the development of an easily administrable (injectable), stable single dose vaccine against SARS-CoV-2 that is less reliant on the cold-chain.

### REFERENCES

1 Rappuoli, R., Mandl, C. W., Black, S. & De Gregorio, E. Vaccines for the twenty-first century society. Nat Rev Immunol 11, 865-872, doi:10.1038/nri3085 (2011).
2 van Panhuis, W. G. et al. Contagious diseases in the United States from 1888 to the present. N Engl J Med 369, 2152-2158, doi:10.1056/NEJMms1215400 (2013).
3 Organization, W. H. Global Vaccine Action Plan 2011-2020. WHO (2013).
4 Moyle, P. M. & Toth, I. Modern subunit vaccines: development, components, and research opportunities. ChemMedChem 8, 360-376, doi:10.1002/cmdc.201200487 (2013).
Vartak, A. & Sucheck, S. J. Recent Advances in Subunit Vaccine Carriers. Vaccines (Basel) 4, doi:10.3390/vaccines4020012 (2016).
6 Reed, S. G., Orr, M. T. & Fox, C. B. Key roles of adjuvants in modern vaccines. Nat Med 19, 1597-1608, doi:10.1038/nm.3409 (2013).
7 Campbell, J. D. Development of the CpG Adjuvant 1018: A Case Study. Methods Mol Biol 1494, 15-27, doi:10.1007/978-1-4939-6445-1_2 (2017).
8 Krieg, A. M. CpG still rocks! Update on an accidental drug. Nucleic Acid Ther 22, 77-89, doi:10.1089/nat.2012.0340 (2012).
9 Ballester. M. et al. Nanoparticle conjugation and pulmonary delivery enhance the protective efficacy of Ag85B and CpG against tuberculosis. Vaccine 29, 6959-6966, doi:10.1016/j.vaccine.2011.07.039 (2011).
10 Sen, D., Forrest, L., Kepler, T. B., Parker, I. & Cahalan, M. D. Selective and site-specific mobilization of dermal dendritic cells and Langerhans cells by Th1- and Th2-polarizing adjuvants. Proc Natl Acad Sci USA 107, 8334-8339, doi:10.1073/pnas.0912817107 (2010).
11 Kuai, R., Ochyl, L. J., Bahjat, K. S., Schwendeman, A. & Moon, J. J. Designer vaccine nanodiscs for personalized cancer immunotherapy. Nat Mater 16, 489-496, doi:10.1038/nmat4822 (2017).
12 Ballester, M. et al. Nanoparticle conjugation enhances the immunomodulatory effects of intranasally delivered CpG in house dust mite-allergic mice. Sci Rep 5, 14274, doi:10.1038/srep14274 (2015).
13 Kuo, T.-Y. et al. Development of CpG-adjuvanted stable prefusion SARS-CoV-2 spike antigen as a subunit vaccine against COVID-19. bioRxiv, 2020.2008.2011.245704, doi:10.1101/2020.08.11.245704 (2020).
14 de Titta, A. et al. Nanoparticle conjugation of CpG enhances adjuvancy for cellular immunity and memory recall at low dose. Proc Natl Acad Sci USA 110, 19902-19907, doi:10.1073/pnas.1313152110 (2013).
15 Buss, C. G. & Bhatia. S. N. Nanoparticle delivery of immunostimulatory oligonucleotides enhances response to checkpoint inhibitor therapeutics. Proc Natl Acad Sci USA 117, 13428-13436, doi:10.1073/pnas.2001569117 (2020).
16 Francica. J. R. et al. Thermoresponsive Polymer Nanoparticles Co-deliver RSV F Trimers with a TLR-7/8 Adjuvant. Bioconjug. Chem. 27, 2372-2385, doi:10.1021/acs.bioconjchem.6b00370 (2016).
17 Lynn, G. M. et al. In vivo characterization of the physicochemical properties of polymer-linked TLR agonists that enhance vaccine immunogenicity. Nat Biotechnol 33, 1201-1210, doi:10.1038/nbt.3371 (2015).
18 Gale, E. C. et al. A Nanoparticle Platform for Improved Potency, Stability, and Adjuvanticity of Poly(I:C). Advanced Therapeutics 3, 1900174, doi:https://doi.org/10.1002/adtp.201900174 (2020).
19 Wilson, D. S. et al. Antigens reversibly conjugated to a polymeric glyco-adjuvant induce protective humoral and cellular immunity. Nat Mater 18, 175-185, doi:10.1038/s41563-018-0256-5 (2019).
Kasturi, S. P. et al. Programming the magnitude and persistence of antibody responses with innate immunity. Nature 470, 543-U136, doi:10.1038/nature09737 (2011).
21 Nederberg, F. et al. Simple approach to stabilized micelles employing miktoarm terpolymers and stereocomplexes with application in paclitaxel delivery. Biomacromolecules 10, 1460-1468, doi:10.1021/bm900056 g (2009).
22 Shi, J., Xiao, Z., Kamaly, N. & Farokhzad, O. C. Self-assembled targeted nanoparticles: evolution of technologies and bench to bedside translation. Acc Chem Res 44, 1123-1134, doi:10.1021/ar200054n (2011).
23 Valencia, P. M. et al. Effects of ligands with different water solubilities on self-assembly and properties of targeted nanoparticles. Biomaterials 32, 6226-6233, doi:10.1016/j.biomaterials.2011.04.078 (2011).
24 Ghasemi, R. et al. mPEG-PLA and PLA-PEG-PLA nanoparticles as new carriers for delivery of recombinant human Growth Hormone (rhGH). Sci Rep 8, 9854, doi:10.1038/s41598-018-28092-8 (2018).
Elsabahy, M. & Wooley, K. L. Design of polymeric nanoparticles for biomedical delivery applications. Chemical Society Reviews 41, 2545-2561. doi:10.1039/C2CS15327K (2012).
26 Tan, J. P. et al. Hierarchical supermolecular structures for sustained drug release. Small 5, 1504-1507, doi:10.1002/smil.200801756 (2009).
27 Irvine, D. J., Aung, A. & Silva, M. Controlling timing and location in vaccines. Adv Drug Deliv Rev, doi:10.1016/j.addr.2020.06.019 (2020).
28 Reddy, S. T., Rehor, A., Schmoekel, H. G., Hubbell, J. A. & Swartz. M. A. In vivo targeting of dendritic cells in lymph nodes with poly(propylene sulfide) nanoparticles. J Control Release 112, 26-34, doi:10.1016/j.jconrel.2006.01.006 (2006).
29 Manolova. V. et al. Nanoparticles target distinct dendritic cell populations according to their size. Eur J Immunol 38, 1404-1413, doi:10.1002/eji.200737984 (2008).
30 Rao, D. A., Forrest, M. L., Alani, A. W., Kwon, G. S. & Robinson. J. R. Biodegradable PLGA based nanoparticles for sustained regional lymphatic drug delivery. J Pharm Sci 99, 2018-2031, doi:10.1002/jps.21970 (2010).
31 Reddy, S. T. et al. Exploiting lymphatic transport and complement activation in nanoparticle vaccines. Nat Biotechnol 25, 1159-1164, doi:10.1038/nbtl332 (2007).
32 Jiang, H., Wang, Q. & Sun, X. Lymph node targeting strategies to improve vaccination efficacy. J Control Release 267, 47-56, doi:10.1016/j.jconrel.2017.08.009 (2017).
33 Schudel, A., Francis, D. M. & Thomas, S. N. Material design for lymph node drug delivery. Nature Reviews Materials 4, 415-428, doi:10.1038/s41578-019-0110-7 (2019).
34 Howard, G. P. et al. Critical size limit of biodegradable nanoparticles for enhanced lymph node trafficking and paracortex penetration. Nano Research 12, 837-844 (2019).
35 Merad, M., Sathe, P., Helft, J., Miller, J. & Mortha, A. The dendritic cell lineage: ontogeny and function of dendritic cells and their subsets in the steady state and the inflamed setting. Annu Rev Immunol 31, 563-604, doi:10.1146/annurev-immunol-020711-074950 (2013).
36 Kato, Y. et al. Multifaceted Effects of Antigen Valency on B Cell Response Composition and Differentiation In Vivo. Immunity 53, 548-563 e548, doi:10.1016/j.immuni.2020.08.001 (2020).
37 Gale, E. C. et al. A Nanoparticle Platform for Improved Potency. Stability, and Adjuvanticity of Poly (I: C). Advanced Therapeutics 3, 1900174 (2020).
38 Tang, S. et al. Versatile Functionalization of Ferritin Nanoparticles by Intein-Mediated Trans-Splicing for Antigen/Adjuvant Co-delivery. Nano Lett 19, 5469-5475, doi:10.1021/acs.nanolett.9b01974 (2019).
39 Boehme, K. W. & Compton. T. Innate sensing of viruses by toll-like receptors. J Virol 78, 7867-7873, doi:10.1128/JVI.78.15.7867-7873.2004 (2004).
40 Tsai, S. J., Black, S. K. & Jewell, C. M. Leveraging the Modularity of Biomaterial Carriers to Tune Immune Responses. Advanced Functional Materials n/a, 2004119, doi:https://doi.org/10.1002/adfm.202004119 (2020).
41 Irvine, D. J., Swartz. M. A. & Szeto, G. L. Engineering synthetic vaccines using cues from natural immunity. Nat Mater 12, 978-90, doi:10.1038/nmat3775 (2013).
42 Demuth, P. C., Garcia-Beltran, W. F., Ai-Ling, M. L., Hammond, P. T. & Irvine, D. J. Composite dissolving microneedles for coordinated control of antigen and adjuvant delivery kinetics in transcutaneous vaccination. Adv Funct Mater 23, 161-172, doi:10.1002/adfm.201201512 (2013).
43 Roth, G. A. et al. Injectable Hydrogels for Sustained Codelivery of Subunit Vaccines Enhance Humoral Immunity. ACS Cent Sci 6, 1800-1812. doi:10.1021/acscentsci.0c00732 (2020).
44 Tam, H. H. et al. Sustained antigen availability during germinal center initiation enhances antibody responses to vaccination. Proc Natl Acad Sci USA 113, E6639-E6648, doi:10.1073/pnas.1606050113 (2016).
45 Boopathy. A. V. et al. Enhancing humoral immunity via sustained-release implantable microneedle patch vaccination. Proc Natl Acad Sci USA 116, 16473-16478, doi:10.1073/pnas.1902179116 (2019).
46 Cirelli, K. M. et al. Slow Delivery Immunization Enhances HIV Neutralizing Antibody and Germinal Center Responses via Modulation of Immunodominance. Cell 177, 1153-1171 el 128, doi:10.1016/j.cell.2019.04.012 (2019).
47 Wu, Y. et al. A Supramolecular Vaccine Platform Based on alpha-Helical Peptide Nanofibers. ACS Biomater Sci Eng 3, 3128-3132, doi:10.1021/acsbiomaterials.7b00561 (2017).
48 Hudalla, G. A. et al. A self-adjuvanting supramolecular vaccine carrying a folded protein antigen Adv Healthc Mater 2, 1114-1119, doi:10.1002/adhm.201200435 (2013).
49 De Koker, S., Hoogenboom, R. & De Geest, B. G. Polymeric multilayer capsules for drug delivery. Chem Soc Rev 41, 2867-2884, doi:10.1039/c2cs15296 g (2012).
50 Appel, E. A. et al. Self-assembled hydrogels utilizing polymer-nanoparticle interactions. Nat Commun 6, 6295, doi:10.1038/ncomms7295 (2015).
51 Appel, E. A., del Barrio, J., Loh, X. J. & Scherman, O. A. Supramolecular polymeric hydrogels. Chem Soc Rev 41, 6195-6214, doi:10.1039/c2cs35264 h (2012).
52 Mann, J. L., Yu, A. C., Agmon, G. & Appel, E. A. Supramolecular polymeric biomaterials. Biomater Sci 6, 10-37, doi:10.1039/c7bm00780a (2017).
53 Stapleton, L. M. et al. Use of a supramolecular polymeric hydrogel as an effective post-operative pericardial adhesion barrier. Nat Biomed Eng 3, 611-620, doi:10.1038/s41551-019-0442-z (2019).
54 Appel, E. A. et al. Exploiting Electrostatic Interactions in Polymer-Nanoparticle Hydrogels. ACS Macro Letters 4, 848-852, doi:10.1021/acsmacrolett.5b00416 (2015).
55 Worbs, T., Hammerschmidt, S. I. & Forster, R. Dendritic cell migration in health and disease. Nat Rev Immunol 17, 30-48, doi:10.1038/nri.2016.116 (2017).
56 Gomati, L., Zanoni, I. & Granucci, F. Dendritic Cells in the Cross Hair for the Generation of Tailored Vaccines. Front Immunol 9, 1484, doi:10.3389/fimmu.2018.01484 (2018).
57 Gurevich, I. et al. Active dissemination of cellular antigens by DCs facilitates CD8(+) T-cell priming in lymph nodes. Eur J Immunol 47, 1802-1818, doi:10.1002/eji.201747042 (2017).
58 Krishnaswamy, J. K. et al. Migratory CD11b(+) conventional dendritic cells induce T follicular helper cell-dependent antibody responses. Sci. Immunol. 2, doi: 10.1126/sciimmunol.aam9169 (2017).
59 Didierlaurent, A. M. et al. Enhancement of adaptive immunity by the human vaccine adjuvant AS01 depends on activated dendritic cells. J Immunol 193, 1920-1930, doi:10.4049/jimmunol.1400948 (2014).
60 Liang, F. et al. Vaccine priming is restricted to draining lymph nodes and controlled by adjuvant-mediated antigen uptake. Sci Transl Med 9, doi:10.1126/scitranslmed.aal2094 (2017).
61 Mesin, L., Ersching. J. & Victora. G. D. Germinal Center B Cell Dynamics. Immunity 45, 471-482, doi:10.1016/j.immuni.2016.09.001 (2016).
62 Maikawa. C. L. et al. Block copolymer composition drives function of self-assembled nanoparticles for delivery of small-molecule cargo. J Polym Sci A Polym Chem 57, 1322-1332, doi:10.1002/pola.29393 (2019).
63 Axelrod, D., Koppel, D. E., Schlessinger, J., Elson, E. & Webb, W. W. Mobility measurement by analysis of fluorescence photobleaching recovery kinetics. Biophysical Journal 16, 1055-1069, doi:https://doi.org/10.1016/S0006-3495(76)85755-4 (1976).
64 Axpe, E. et al. A Multiscale Model for Solute Diffusion in Hydrogels. Macromolecules 52, 6889-6897, doi:10.1021/acs.macromol.9b00753 (2019).
65 Neuman, B. W. et al. Supramolecular architecture of severe acute respiratory syndrome coronavirus revealed by electron cryomicroscopy. J Virol 80, 7918-7928, doi:10.1128/JVI.00645-06 (2006).
66 Powell, A. E. et al. A single immunization with spike-functionalized ferritin vaccines elicits neutralizing antibody responses against SARS-CoV-2 in mice. bioRxiv, doi:10.1101/2020.08.28.272518 (2020).
67 Smith, A. A. A. et al Nanoparticles Presenting Potent TLR7/8 Agonists Enhance Anti-PD-L1 Immunotherapy in Cancer Treatment. Biomacromolecules 21, 3704-3712, doi:10.1021/acs.biomac.0c00812 (2020).
68 Schubert, S., Delaney. J. J. T. & Schubert, U. S. Nanoprecipitation and nanoformulation of polymers: from history to powerful possibilities beyond poly(lactic acid).
Soft Matter 7, 1581-1588, doi:10.1039/C0SM00862A (2011).
69 Almoustafa, H. A., Alshawsh, M. A. & Chik, Z. Technical aspects of preparing PEG-PLGA nanoparticles as carrier for chemotherapeutic agents by nanoprecipitation method. Int J Pharm 533, 275-284, doi:10.1016/j.ijpharm.2017.09.054 (2017).
70 Pustulka, K. M. et al. Flash nanoprecipitation: particle structure and stability. Mol Pharm 10, 4367-4377, doi:10.1021/mp400337f (2013).
71 Nuhn, L. et al. Potent anti-viral vaccine adjuvant based on pH-degradable nanogels with covalently linked small molecule imidazoquinoline TLR7/8 agonist. Biomaterials 178, 643-651, doi:10.1016/j.biomaterials.2018.03.026 (2018).
72 Klein, F. et al. Somatic mutations of the immunoglobulin framework are generally required for broad and potent HIV-1 neutralization. Cell 153, 126-138, doi:10.1016/j.cell.2013.03.018 (2013).
73 Larson, R. G. The structure and rheology of complex fluids. (1999).
74 Fenton, O. S. et al. Injectable Polymer-Nanoparticle Hydrogels for Local Immune Cell Recruitment. Biomacromolecules 20, 4430-4436, doi:10.1021/acs.biomac.9b01129 (2019).
75 Klein, D. C. G., Latz, E., Espevik, T. & Stokke, B. T. Higher order structure of short immunostimulatory oligonucleotides studied by atomic force microscopy. Ultramicroscopy 110, 689-693, doi:https://doi.org/10.1016/j.ultramic.2010.02.033 (2010).

### EXEMPLARY EMBODIMENTS

Exemplary embodiments provided in accordance with the presently disclosed subject matter include, but are not limited to, the claims and the following embodiments:
1. A nanoparticle comprising a polymer and a plurality of Toll-like receptor (TLR) agonist moieties conjugated to the polymer, wherein the plurality of TLR agonist moieties is present on the surface of the nanoparticle.
2. The nanoparticle of embodiment 1, wherein the plurality of TLR agonist moieties is a plurality of TLR7/8 agonist moieties.
3. The nanoparticle of embodiment 2, wherein the plurality of TLR agonist moieties comprises a 1H-imidazo[4,5-c]quinolone core structure.
4. The nanoparticle of embodiment 3, wherein the plurality of TLR agonist moieties comprises resiquimod (R848), imiquimod, gardiquimod, or mixtures thereof.
5. The nanoparticle of embodiment 3, wherein the plurality of TLR7/8 agonist moieties comprises a plurality of N-(4-((4-amino-2-(ethoxymethyl)-1H-imidazo[4,5-c]quinolin-1-yl)methyl)benzyl)-3-(prop-2-yn-1-yloxy)propanamide moieties.
6. The nanoparticle of embodiment 1, wherein the plurality of TLR agonist moieties is a plurality of TLR9 agonist moieties.
7. The nanoparticle of embodiment 6, wherein the plurality of TLR9 agonist moieties comprises a plurality of cytidine-phosphate-guanosine (CpG) moieties.
8. The nanoparticle of any one of embodiments 1 to 7, wherein the polymer comprises poly(ethylene glycol)-b-poly(lactic acid) (PEG-PLA).
9. The nanoparticle of any one of embodiments 1 to 8, wherein the nanoparticle is up to about 200 nm in diameter.
10. The nanoparticle of embodiment 9, wherein the nanoparticle is about 50 nm in diameter.
11. The nanoparticle of embodiment 9, wherein the nanoparticle is about 30 nm in diameter.
12. The nanoparticle of any one of embodiments 1 to 11, wherein the nanoparticle further comprises a plurality of mannose moieties conjugated to the polymer.
13. A method of producing the nanoparticle of any one of embodiments 1 to 12, the method comprising:
(a) conjugating the polymer with the plurality of TLR agonist moieties to form a conjugated polymer;
(b) mixing an amount of the conjugated polymer and an amount of unconjugated polymer at a ratio to achieve a target density of the plurality of TLR agonist moieties on the surface of the nanoparticle; and
(c) precipitating the nanoparticle from the mixture.
14. A hydrogel comprising the nanoparticle of any one of embodiments 1 to 12.
15. The hydrogel of embodiment 14, wherein the hydrogel comprises optionally hydrophobically-modified hydroxypropyl methylcellulose (HPMC).
16. A vaccine comprising the nanoparticle of any one of embodiments 1 to 12, and one or more subunit antigens.
17. A vaccine comprising the hydrogel of embodiment 14 or 15, wherein the hydrogel comprises the nanoparticle and one or more subunit antigens.
18. The vaccine of embodiment 16 or 17, wherein the antigen comprises a viral antigen, a bacterial antigen, a fungal antigen, or a protozoan antigen.
19. The vaccine of embodiment 18, wherein the viral antigen comprises a SARS-CoV-2 subunit antigen.
20. A method for inducing an antigen-specific humoral immune response in a subject, the method comprising administering the vaccine of any one of embodiments 16 to 19.
21. A method for enhancing cancer immunotherapy in a subject, the method comprising administering the nanoparticle of any one of embodiments 1 to 12 or the hydrogel of embodiment 14 or 15.
22. The method of embodiment 21, wherein the nanoparticle or the hydrogel is co-administered with an immune checkpoint inhibitor, an immunomodulatory molecule, or a combination thereof.
23. The method of embodiment 22, wherein the immune checkpoint inhibitor is a checkpoint antibody.
24. The method of embodiment 22 or 23, wherein the immune checkpoint inhibitor is an antibody that prevents interactions of CTLA4/(CD80/CD86) and PD1/PD-L1.
25. The method of any one of embodiments 22 to 24, wherein the immunomodulatory molecule is a cytokine or chemokine.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A nanoparticle comprising a polymer and a plurality of Toll-like receptor (TLR) agonist moieties conjugated to the polymer, wherein the plurality of TLR agonist moieties is present on the surface of the nanoparticle.

2. The nanoparticle of claim 1, wherein the plurality of TLR agonist moieties is a plurality of TLR7/8 agonist moieties.

3. The nanoparticle of claim 2, wherein the plurality of TLR agonist moieties comprises a 1H-imidazo[4,5-c]quinolone core structure.

4. The nanoparticle of claim 3, wherein the plurality of TLR agonist moieties comprises resiquimod (R848), imiquimod, gardiquimod, or mixtures thereof.

5. The nanoparticle of claim 3, wherein the plurality of TLR7/8 agonist moieties comprises a plurality of N-(4-((4-amino-2-(ethoxymethyl)-1H-imidazo[4,5-c]quinolin-1-yl)methyl)benzyl)-3-(prop-2-yn-1-yloxy)propanamide moieties.

6. The nanoparticle of claim 1, wherein the plurality of TLR agonist moieties is a plurality of TLR9 agonist moieties.

7. The nanoparticle of claim 6, wherein the plurality of TLR9 agonist moieties comprises a plurality of cytidine-phosphate-guanosine (CpG) moieties.

8. The nanoparticle of claim 1, wherein the polymer comprises poly(ethylene glycol)-b-poly(lactic acid) (PEG-PLA).

9. The nanoparticle of claim 1, wherein the nanoparticle is up to about 200 nm in diameter.

10. The nanoparticle of claim 9, wherein the nanoparticle is about 50 nm in diameter.

11. The nanoparticle of claim 9, wherein the nanoparticle is about 30 nm in diameter.

12. The nanoparticle of claim 1, wherein the nanoparticle further comprises a plurality of mannose moieties conjugated to the polymer.

13. A method of producing the nanoparticle of claim 1, the method comprising:
(a) conjugating the polymer with the plurality of TLR agonist moieties to form a conjugated polymer;
(b) mixing an amount of the conjugated polymer and an amount of unconjugated polymer at a ratio to achieve a target density of the plurality of TLR agonist moieties on the surface of the nanoparticle; and
(c) precipitating the nanoparticle from the mixture.

14. A hydrogel comprising the nanoparticle of claim 1.

15. The hydrogel of claim 14, wherein the hydrogel comprises optionally hydrophobically-modified hydroxypropyl methylcellulose (HPMC).

16. A vaccine comprising the nanoparticle of claim 1, and one or more subunit antigens.

17. A vaccine comprising the hydrogel of claim 14, wherein the hydrogel comprises the nanoparticle and one or more subunit antigens.

18. The vaccine of claim 16 or 17, wherein the antigen comprises a viral antigen, a bacterial antigen, a fungal antigen, or a protozoan antigen.

19. The vaccine of claim 18, wherein the viral antigen comprises a SARS-CoV-2 subunit antigen.

20. A method for inducing an antigen-specific humoral immune response in a subject, the method comprising administering the vaccine of claim 16 or 17.

21. A method for enhancing cancer immunotherapy in a subject, the method comprising administering the nanoparticle of claim 1 or the hydrogel of claim 14.

22. The method of claim 21, wherein the nanoparticle or the hydrogel is co-administered with an immune checkpoint inhibitor, an immunomodulatory molecule, or a combination thereof.

23. The method of claim 22, wherein the immune checkpoint inhibitor is a checkpoint antibody.

24. The method of claim 22, wherein the immune checkpoint inhibitor is an antibody that prevents interactions of CTLA4/(CD80/CD86) and PD1/PD-L1.

25. The method of claim 22, wherein the immunomodulatory molecule is a cytokine or chemokine.
